(19) United States
(12) Patent Application Publication
(10) Pub. No.: US 2026/0199474 A1
(43) Pub. Date: Jul. 16, 2026

(54) Doxorubicin-Based Nanomedicines for Combined Chemo-Phototherapy of Cancer

(71) Applicant: Board of Trustees of the University of Arkansas, Little Rock, AR (US)

(72) Inventor: Noureen Siraj, Little Rock, AR (US)

(21) Appl. No.: 19/342,132

(22) Filed: Sep. 26, 2025

Related U.S. Application Data
(60) Provisional application No. 63/745,497, filed on Jan. 15, 2025.

(51) Int. Cl. A61K 41/00; A61K 47/54; A61P 35/00

(52) U.S. Cl. CPC A61K 41/0052; A61K 47/545; A61P 35/00

(57) ABSTRACT

A compound for treating cancer including doxorubicin electrostatically attached to indocyanine green, IR783, or IR820. A method of treating cancer in a subject in need thereof is also disclosed, which includes administering to the subject a pharmaceutically effective amount of a compound including doxorubicin electrostatically attached to indocyanine green, IR783, or IR820, and exposing the subject to near-infrared light.

### CROSS-REFERENCE TO RELATED APPLICATIONS

This application claims the benefit of U.S. Provisional Application No. 63/745,497, entitled “Ionic Materials-Based Combination Nanomedicines” and filed on Jan. 15, 2025. The complete disclosure of said patent application is hereby incorporated by reference.

### STATEMENT REGARDING FEDERALLY SPONSORED RESEARCH OR DEVELOPMENT

This invention was made with Government support under Grant No. RII Track 4-183304 awarded by the National Science Foundation and Grant No. P20 GM103429 awarded by the National Institutes of Health. The Government has certain rights in the invention.

### BACKGROUND OF THE INVENTION

Despite improvements in prevention, early detection, and available treatment options, the latest annual reports from the National Cancer Institutes for cancer statistics revealed an increasing trend in cancer incidence and mortality rate. Numerous cancer types, including breast cancer, melanoma, kidney, and pancreatic cancer have been treated using conventional methods such as surgery, chemotherapy, radiation therapy, and immunotherapy. However, some of these strategies are limited due to invasiveness, side effects imposed from lack of specificity, selectivity, and multidrug resistance (MDR). Recently, non-invasive near infrared (NIR) mediated phototherapy techniques such as photothermal and photodynamic therapies (PTT and PDT) have been explored for cancer treatment. PTT involves the use of photothermal agents (PTAs) to produce heat upon absorption of light resulting in hyperthermia that leads to cancerous cell death. The discovery that the heat created by PTAs also lowers interstitial fluid pressure and enhances blood circulation, in addition to direct killing of tumor cells, makes PTT a promising treatment option.

Cyanine dyes like indocyanine green (ICG), an FDA approved drug are studied for cancer treatment, imaging diagnosis, and drug delivery applications. The use of other NIR cyanine dyes such as IR783 and IR820 is also gaining significant attention for cancer application. Normally, dyes that absorb visible wavelength region of electromagnetic radiation spectrum are used for treatment of skin infections. Whereas PTAs that absorb light in the therapeutic NIR window (above 780 nm), exhibit deeper penetration depth in body tissues. Thus, making it favorable to treat deeply seated malignancies. Despite their selectivity, ICG exhibits concentration-dependent aggregation, light sensitivity, and short blood half-life of 2-4 mins in aqueous environments hindering its use in medicine. Similarly, the hydrophilic property and shelf life of IR783 and IR820 are also disadvantages for direct in vitro and in vivo use. These NIR dyes are poorly absorbed by the body due to their hydrophilic nature, which causes a rapid excretion. Therefore, these soluble PTAs have been modified mostly using inorganic and organic nanocarriers. However, some of these nanocarriers are imposing unwanted processes such as absorption, distribution, metabolism and excretion (ADME profile) of the drug when used for biological purposes. Thus, there is a need for new simple and economical strategies to optimize the performance of NIR dyes. Among others, combination therapy and carrier free nanomedicine based on NIR dyes are promising approaches to develop ideal drugs.

Combination therapy involves combining more than one therapeutic mechanism in a drug to treat tumor effectively. Moreover, combination therapy can address several drawbacks associated with single therapy such as MDR, solubility and dosage-related problems. Combination medicines can perform better than single drug owing to the powerful synergistic effect of multi-modal therapeutics. Studies have revealed that PTT and chemotherapy complement each other by enhancing drug' performance in the combination form than the individual parent drugs. Most combination therapeutics involve active agents developed via costly, multiple step, and complicated organic synthesis commonly known for a very low product yield. Thus, it is utmost importance to implement new economical and simple synthesis strategies for the development of combination drugs.

Recently, nanotechnology involving the use of nanocarriers and nanovehicles has served as a promising approach for cancer treatment. Tumor cells have poorly aligned and leaky vessels, thus making the accumulation of nanosized drugs to have better penetration and retention capabilities within the tumor. Additionally, the use of nanoparticle for drug delivery offers benefit such as targeting certain cells and safeguarding sensitive therapeutics against premature deterioration, all of which can help mitigate negative effect. Unfortunately, most studied nanomaterials are metal-based such as gold, and iron oxide nanoparticles having significant limitations in the biological system owning to their non-biodegradable and non-biocompatible features. These nanomaterials require further appropriate surface functionalization and special synthetic method in order to be used for clinical application. Thus, carrier-free nanodrugs composed of soft organic materials that are highly biocompatible, and biodegradable is an excellent choice for cancer treatment at present.

Herein, three new chemo-PTT combination ionic nanomedicines (INMs) are designed using FDA approved chemo drug (Doxorubicin hydrochloride i.e., DOX.HCl) and NIR dye (ICG, IR783 and IR820) by employing ionic liquid (IL) chemistry. The simple and economical synthesis of ionic materials (IMs) with distinct properties are useful for biological applications. Their excellent thermal stability, high photostability, and most importantly ease of tunability, are few of these qualities that make the IL-approach simple to use in the synthesis of chemo-PTT combination IMs. The selected FDA approved chemo (DOX) and photothermal therapeutic agents in the INMs form were used for the treatment MCF-7 cancer cells. DOX, is known for its DNA-binding properties, topoisomerase II inhibition and production of free radicals capable of causing programmed cell death (apoptosis). It is anticipated that the use of combination therapy will significantly minimize the dose of the chemo and PTT drugs, thereby addressing multidrug resistance (MDR), minimizing side effects to normal cells, producing a synergistic effect of both chemo and PTT drug with improved therapeutic efficacy. In addition, the heat produced by the PTT drug upon laser irradiation can also contribute to better drug uptake by the cells, thereby boosting the therapeutic potency of the chemo-PTT combination nanodrug. The newly synthesized distinct chemo-PTT INMs were examined for their physico-chemical properties, photophysical characteristics, light to heat efficiency, singlet oxygen quantum yield, cellular uptake, in vitro cytotoxicity and apoptosis mechanism to provide insight on their potential as combination anticancer nanomedicines. As far as we know, this is the first report employing ILs chemistry for developing combination nanomedicine using FDA approved drug-DOX.HCl.

### BRIEF SUMMARY OF THE INVENTION

Synergistic combination therapy approach offers lots of options for delivery of materials with anti-cancer properties which is a very auspicious strategy to treat a variety of malignant lesions with enhanced therapeutic efficacy. The current study involves a detailed investigation of combination INMs where chemotherapeutic drug is coupled with a photothermal agent to attain dual mechanisms (chemotherapy (chemo) and photothermal therapy (PTT) to improve the drug's efficacy. An FDA approved Doxorubicin (DOX) is electrostatically attached with a near infrared cyanine dye (ICG, IR783, and IR820) which serves as a PTT drug using IL chemistry to develop three IM-based chemo-PTT drugs. Carrier free INMs are derived from IM. Photophysical properties of the developed combination IMs and their INMs were studied in depth. The phototherapeutic efficiency of the combination drugs was evaluated by measuring photothermal conversion efficiency and singlet oxygen quantum yield. The improved photophysical properties of the combination nanomedicines in comparison to their parent compounds significantly enhanced INMs' photothermal efficiency. Cellular uptake, dark and light toxicity studies, and cell death mechanism of the chemo-PTT nanoparticles were also studied in vitro. The combination INMs exhibited enhanced cytotoxicity as compared to their respective parent compounds. Moreover, the apoptosis cell death mechanism was almost doubled for combination nanomedicine than the free DOX which is attributed to enhanced cellular uptake. Examination of the combination index and improved in vitro cytotoxicity results revealed a great synergy between chemo and PTT drugs in the developed combination nanomedicines.

We have studied the endocytic mechanisms as well as subcellular localization for three carrier-free chemotherapeutic-photothermal (chemo-PTT) combination INMs composed of DOX and an NIR dye (ICG, IR820, or IR783). This study aims to understand the cellular basis for enhanced toxicity results of these combination nanomedicines towards MCF-7 breast cancer cells. The active transport mechanism of INMs, unlike free DOX which is known to employ passive transport, was validated by conducting temperature-dependent cellular uptake of drug in MCF-7 cells using confocal microscopy. The internalization pathway of these INMs were further probed in the presence and absence of different endocytosis inhibitors. Detailed examination of mode of entry of the carrier-free INMs in MCF-7 cells revealed that they are primarily internalized through clathrin-mediated endocytosis. In addition, time-dependent subcellular localization studies were also investigated. Examination of time-dependent confocal images indicated that the INMs targeted multiple organelles, in contrast to free DOX which primarily targets the nucleus. Collectively, the high cellular endocytic uptake in cancerous cells due to enhance permeability and retention effect (EPR effect) and the multimode targeting ability demonstrated the main reason of the low IC₅₀ value (the high cytotoxicity) of these carrier free INMs as compared to their respective parent chemo and PTT drugs.

In one embodiment, the present invention is directed to a compound for treating cancer using two different mechanisms and includes DOX electrostatically attached to indocyanine green, IR783, or IR820. The new compound exhibited the nanoparticle morphology in aqueous media. The combination nanomaterials showed high toxicity towards cancer cells due to their nanoparticle morphology as well as improved photophysical properties, which enhanced their phototherapeutic activities. In addition, the combination INMs drug exhibited high cellular uptake and synergistic enhanced chemotherapeutic and phototherapeutic activities.

In another embodiment, the present invention is directed to a method of treating cancer in a subject in need thereof is also disclosed, which includes administering to the subject a pharmaceutically effective amount of a compound including DOX electrostatically attached to indocyanine green, IR783, or IR820. The combination nanodrug exhibited enhanced tumor activity and therefore four time low dose is needed to treat tumor selectivity, which also minimize the side effect of DOX hydrochloride used alone. For phototherapy, laser radiation exposure towards cancer cells also eliminated only cancerous cells. The laser power of 1 Watt/cm² and 0.5 Watt/cm² for a 1 min daily for three days after drug administration significantly reduced cancer cells.

As shown herein, the three combination INMs (DOX-ICG, DOX-IR783, and DOX-IR820) are useful as nanoparticles in cancer studies. Higher cellular uptake of INMs compared to their original parent compounds due to their nanoparticle morphology showed enhanced chemotherapeutic and phototherapeutics activities. Improved photophysical properties of INMs showed enhanced phototherapeutic activities of INMs compared to original NIR dyes. The enhanced phototherapeutic activity of the NIR component of the INMs is due to Forster resonance energy transfer (FRET) mechanism between the DOX cation and NIR anion in INMs. The lower IC50 value of INMs in dark is attained due to high cellular uptake as compared to their parent Doxorubicin hydrochloride which also address the minimum side effect of INMs, since less dose of INMs is needed to treat cancer.

These and other features, objects and advantages of the present invention will become better understood from a consideration of the following detailed description of the preferred embodiments and appended claims in conjunction with the drawings as described following:

### BRIEF DESCRIPTION OF THE DRAWINGS

FIG. 1A is the chemical reaction showing the synthesis of ionic material using ion-exchange reaction. FIG. 1B is a schematic showing the ionic nanomaterial synthesis.

FIGS. 2A-2C are TEM images for [DOX][ICG]INM (FIG. 2A), [DOX][IR783]INM (FIG. 2B), and [DOX][IR820]INM (FIG. 2C).

FIG. 3A is a normalized absorption spectra of DOX and three chemo-PTT combination INMs in water. FIG. 3B is a fluorescence emission spectra of NIR dyes and chemo-PTT combination INMs in water at an excitation wavelength of 710 nm.

FIG. 4 is a graph showing cellular uptake of three ionic nanomaterials in relation to free soluble drugs after 6 h incubation of a 60000 pmol drug in MCF-7 cancer cells. Data are presented as mean±SD (n=3). Data are presented as mean SD (n=3). (*p<0.05, **p<0.01, ***p<0.005).

FIG. 5A is a graph showing cell viability results for varying concentrations of parent NaICG and doxorubicin-based drugs in MCF-7 cancer cells treated for 24 h in the dark. FIG. 5B is a graph showing cell viability results for NaICG and [DOX][ICG]INMs in MCF-7 cancer cells incubated for 6 h in MCF-7 cells and irradiated with 808 nm laser (1 W cm⁻²) for 5 min. Using a two-tailed student's t test, p-values are determined and are presented as *p<0.05, **p<0.01, and ***p<0.005.

FIG. 6 is confocal microscopy imaging of caspase 3/7 activation on MCF-7 cells incubated with 5 μM DOX and three ionic nanomaterials for 6 h. Scale bar is 10 μm.

FIG. 7A-7E are YO-PRO/propidium iodide (PI) staining results for MCF-7 cells treated with INMs and DOX after 48 h of drug incubation. Numbers in quadrants show percentages (%) of the total cell populations. FIG. 7F is a bar graph showing data from flow cytometry experiments. Error bars are presented as mean±standard deviation (SD).

FIG. 8 is a table showing fluorescence quantum yield, radiative and non radiate rate for parent NIR dyes and newly developed combination nanomedicines.

FIG. 9 is a table showing the light to heat conversion efficiency (PTT activity) for original NIR dyes and recently developed combination nanomedicines.

FIG. 10 is a table showing half-maximal inhibitory concentration of parent compounds and INMs in dark (chemotherapeutic activity) as well as under laser light (PTT+ chemo activity) exposure

FIGS. 11A-11E are ESI-MS of [DOX][ICG] in positive ion mode (FIG. 11A), [DOX][ICG] in negative ion mode (FIG. 11B) [DOX][IR820] in positive ion mode (FIG. 11C), [DOX][IR820] in negative ion mode (FIG. 11D), [DOX][IR783] in positive ion mode (FIG. 11E), and [DOX][IR783] in negative ion mode (FIG. 11F).

FIGS. 12A-12C are NMR spectrum of [DOX][ICG](FIG. 12A), [DOX][IR820](FIG. 12B), and [DOX][IR783](FIG. 12C).

FIG. 13 is the chemical equation for the synthesis of [DOX][IR783]ionic material using ion-exchange reaction.

FIG. 14 is the chemical equation for the synthesis of [DOX][IR820]ionic material using ion-exchange reaction.

FIG. 15 is a table showing the melting point values of the synthesized IMs as compared to the free NIR dyes.

FIG. 16 is a table showing Zeta potential and Polydispersity index for all INMs.

FIGS. 17A-17C are time-dependent DLS plots for [DOX][IR820]INMs at 0th hr (FIG. 17A), 4 hr (FIG. 17B), and 24 hr (FIG. 17C).

FIGS. 18A-18C are normalized absorbance of [DOX][ICG]INMs and NaICG in water (FIG. 18A), [DOX][IR820]INMs and NaIR820 in water (FIG. 18B), and [DOX][IR783]INMs and NaIR783 in water (FIG. 18C).

FIGS. 19A-19C show normalized absorption spectra in ethanol for DOX, NaICG and [DOX][ICG](FIG. 19A), DOX, NaIR820 and [DOX][IR820](FIG. 19B), and DOX, NaIR783 and [DOX][IR783](FIG. 19C).

FIG. 20 is a table showing molar absorptivity values for parent dye, INMs and IMs in water and ethanol respectively.

FIGS. 21A-21C are absorption spectra in phosphate buffer saline (PBS), cell media (CM) and water for NaICG and [DOX][ICG](FIG. 21A), NaIR820 and [DOX][IR820](FIG. 21B), and NaIR783 and [DOX][IR783](FIG. 21C).

FIGS. 22A-22D are fluorescence emission spectra of chemo-PTT combination IMs in ethanol at an excitation wavelength of 480 nm (FIG. 22A), while all fluorescence spectra in FIGS. 22B, 22C and 22D are recorded in ethanol at an excitation wavelength of 710 nm, NaICG and [DOX][ICG]IMs (FIG. 22B), NaIR820 and [DOX][IR820]IMs (FIG. 22C), and NaIR783 and [DOX][IR783]IMs (FIG. 22D). FIG. 22E is a fluorescence spectra of DOX and chemo-PTT combination INMs in water (nanoparticles) at an excitation wavelength of 480 nm.

FIGS. 23A-23F are normalized fluorescence emission spectra of DOX and NaICG absorption spectra in water (FIG. 23A), DOX and NaIR820 absorption spectra in water (FIG. 23B), DOX and NaIR783 absorption spectra in water (FIG. 23C), DOX and NaICG absorption spectra in ethanol (FIG. 23D), DOX and NaIR820 absorption spectra in ethanol (FIG. 23E), and DOX and NaIR783 absorption spectra in ethanol (FIG. 23F).

FIG. 24 is a table showing FRET efficiency for all INMs in the presence of DOX (donor).

FIG. 25 is a table showing FRET efficiency for all IMs in the presence of DOX (donor).

FIG. 26 is a table showing fluorescence quantum yield (φF), radiative rate (krad), and non-radiative rate (knon-rad) of all IMs in ethanol at an excitation wavelength of 710 nm.

FIG. 27 is a table showing fluorescence quantum yield (φF) of INMs and IMs in water and ethanol at an excitation wavelength of 480 nm. DOX was used as reference with a reported literature fluorescence quantum yield of 9%³.

FIG. 28 is a table showing fluorescence lifetimes of 10 μM combination drugs in ethanol recorded at 455 nm excitation/590 nm emission.

FIGS. 29A-29C are photostability results for [DOX][ICG]INM and IM in water and ethanol (FIG. 29A), [DOX][IR820]INM and IM in water and ethanol (FIG. 29B), and [DOX][IR783]INM and IM in water and ethanol (FIG. 29C).

FIGS. 30A-30C are light to heat conversion efficiency curves for [DOX][ICG]INMs and NaICG (FIG. 30A), [DOX][IR783]INMs sand NaIR783 (FIG. 30B), and [DOX][IR820]INMs and NaIR820 (FIG. 30C) in cell media conducted for 5-10 mins. Error bars are presented as mean+standard deviation (SD).

FIGS. 31A-31C are light to heat conversion efficiency curves for [DOX][ICG]INMs and NaICG (FIG. 31A), [DOX][IR820]INMs and NaIR820 (FIG. 31B), [DOX][IR783]INMs and NaIR783 in pure water conducted for 5-10 mins (FIG. 31C).

FIG. 31D is an extended light to heat conversion efficiency curve for [DOX][ICG] and NaICG in water conducted for 8-16 mins. Error bars are presented as mean+standard deviation (SD).

FIGS. 32A-32C are light to heat conversion efficiency curve for [DOX][ICG]INMs and NaICG (FIG. 32A), [DOX][IR820]INMs and NaIR820 (FIG. 32B), and [DOX][IR783]INMs and NaIR783 (FIG. 32C) in PBS conducted for 5-10 mins. Error bars are presented as mean+standard deviation (SD).

FIG. 33 shows photothermal efficiency for all INMs and parent dyes in pure water and PBS.

FIG. 34 is a graph showing photodegradation of DPBF upon increasing irradiation time in the presence of [DOX][ICG] in ethanol.

FIG. 35 is a graph showing absorbance of DPBF probe (411 nm) in ethanol after irradiation with 808 nm laser over increased time.

FIG. 36A-36C are graphs showing SOQY results in ethanol for NaICG and [DOX][ICG](FIG. 36A), NaIR820 and [DOX][IR820](FIG. 36B), and NaIR783 and [DOX][IR783](FIG. 36C).

FIG. 37A-37C are graphs showing SOQY results in water for NaICG and [DOX][ICG](FIG. 37A), NaIR820 and [DOX][IR820](FIG. 37B), and NaIR783 and [DOX][IR783](FIG. 37C).

FIG. 38 is a table showing SOQY for the INMs and IMs respectively in water and ethanol respectively.

FIG. 39A-39B are graphs showing time-dependent cellular uptake for [DOX][ICG](FIG. 39A) and [DOX][IR820](FIG. 39B) on MCF-7 cells.

FIG. 40A-40D are graphs showing cell viability results for varying concentrations of NaIR820 and doxorubicin-based drugs treated for 24 hr in the dark (FIG. 40A), cell viability results for NaIR820 and [DOX][IR820]INMs incubated for 6 hr and irradiated with 808 nm laser (1 Wcm−2) for 5 min. (FIG. 40B), cell viability results for varying concentrations of NaIR783 and doxorubicin-based drugs treated for 24 hr in the dark (FIG. 40C), and cell viability results for NaIR783 and [DOX][IR783] on MCF-7 breast cancer cell line treated for 6 hr and exposed to light (FIG. 40D). p values are determined using two-tailed student's t-test and are reported as *p<0.05, **p<0.01, ***p<0.005.

FIG. 41 is a graph showing cell viability results of parent PTT drugs on MCF-7 cell lines incubated for 6 hr and exposed to 1 Wcm−2 of 808 nm laser radiation for 5 min. p values are determined using two-tailed student's t-test and are reported as *p<0.05, **p<0.01, ***p<0.005.

FIG. 42 shows YO-PRO/propidium-iodide (PI) staining results for MCF-7 cells treated with [DOX][IR783]INMs and DOX after 6 hr drug incubation. Numbers in quadrants show percentages (%) of total cell populations.

FIG. 43A shows the chemical structure of NIR dyes. FIG. 43B shows the chemical structure of DOX chemotherapeutic drug used for IMs synthesis. FIG. 43C shows the synthesis scheme for the three chemo-PTT combination IMs.

FIG. 44 are confocal microscopy images showing the internalization of DOX and DOX-based INMs recorded at two different temperatures (4 and 37° C.) in MCF-7 cells. Drugs were introduced at 5 μM concentration and incubated for 1 h. Scale bar=10 μm.

FIG. 45 is a table showing a list of inhibitors responsible to block different endocytosis pathways

FIG. 46 shows confocal microscopy images of macropinocytosis inhibitor-treated MCF-7 cells incubated with [DOX][IR820]INMs. Cells were incubated with amiloride and imipramine at 5 μM and 12.6 μM, respectively, prior to drug treatment. Scale bar represents 10 μm.

FIG. 47 shows confocal microscopy images of [DOX][IR820]INMs-treated MCF-7 cells in the presence of filipin III, a CVME-related inhibitor, introduced at a 4.6 mM concentration. Scale bar represents 10 μm.

FIG. 48A-48C are confocal microscopy images of CME-related inhibitor-treated MCF-7 cells incubated with [DOX][IR820]INMs. M #CD (FIG. 5A), sucrose (FIG. 5B), and chlorpromazine (FIG. 5C). Cells were incubated with M #CD, sucrose, and chlorpromazine at 2.5 μM, 0.3 mM, and 21.9 μM, respectively. Scale bar represents 10 μm.

FIG. 49 is a graph showing quantitative measurement of the average fluorescence emission of three INMs in the presence of chlorpromazine, filipin III, imipramine, and chloroquine known for CME, CVME, macropinocytosis, and CME inhibition, respectively.

FIG. 50 is a graph showing cell viability of [DOX][IR820]INMs in MCF-7 cells in the presence of different endocytosis inhibitors preincubated for 2 h at 37° C. prior to drug exposure for 24 h. Cells were exposed to [DOX][IR820]INMs at 0.74 μM. The results are represented as mean±SD (n=3), statistically significant p s 0.05 (*) was evaluated using the student t test.

FIG. 51 shows time-dependent subcellular localization (1 and 6 h) of MCF-7 cell lines treated with 5 μM DOX or chemo-PTT combination INMs, post-treated with DAPI and LAMP 2 antibodies. Scale bars represent 10 μm.

FIG. 52 is a graph showing quantitative measurement of DOX and DOX-based INMs in the nucleus and LAMP 2.

FIG. 53 is a table identifying different endocytosis inhibitors and their concentrations.

FIG. 54 shows cell viability endocytic inhibitors in MCF-7 cells pre-incubated for 2 hr at 37° C. prior to cell media introduction for 24 hr. Inhibitor concentrations are reported in Table S1 above. The results are represented as mean+SD (n=3), statistically significant p s 0.05 (*) was evaluated using the student t-test.

FIG. 55 is a bar graph representing the IC50 values for DOX and three chemo-PTT combination INMs on MCF-7 cells incubated for 24 hr. Bar graph obtained from IC50 values generated from previous findings.

FIG. 56 are confocal microscopy images of MCF-7 cells treated with [DOX][IR783]INMs in the presence of macropinocytosis inhibitors introduced at 5 μM and 12.6 μM for amiloride and imipramine. Scale bar represents 10 μm.

FIGS. 57A-57C are confocal microscopy images of CME-related inhibitor treated MCF-7 cells incubated with [DOX][IR783]INMs. MβCD (FIG. 57A), chlorpromazine (FIG. 57B), and sucrose (FIG. 57C). MβCD, chlorpromazine and sucrose introduced at 2.5 μM, 21.9 μM and 0.3 mM respectively. Scale bar represents 10 μm.

FIG. 58 are confocal microscopy images of [DOX][IR783]INMs treated MCF-7 cells in the presence of filipin III at 4.6 mM. Scale bar represents 10 μm.

FIG. 59 are confocal microscopy images of MCF-7 cells treated with [DOX][ICG]INMs pre-treated with macropinocytosis inhibitors introduced at a concentration of 5 μM for amiloride and 12.6 μM for imipramine. Scale bar represents 10 μm.

FIG. 60 are confocal microscopy images of [DOX][ICG]INMs treated MCF-7 cells in the presence of Filipin III inhibitor introduced at 4.6 mM. Scale bar represents 10 μm.

FIG. 61A-61B are Confocal microscopy images of CME-related inhibitor treated MCF-7 cells incubated with [DOX][ICG]INMs. MβCD (FIG. 61A), chlorpromazine (FIG. 61B), and sucrose (FIG. 61C). MβCD, chlorpromazine and sucrose introduced at 2.5 μM, 21.9 μM and 0.3 mM respectively. Scale bar represents 10 μm.

FIG. 62 are confocal microscopy images of [DOX][IR820], [DOX][IR783] and [DOX][ICG]INMs pretreated separately with chloroquine and AEBSF at 100 μM and 0.5 mM respectively. Scale bar represents 10 μm.

FIG. 63 is a graph showing cell viability of [DOX][IR783]INMs in MCF-7 cells in the presence of different endocytosis inhibitors pre-incubated for 2 hr at 37° C. prior to drug introduction for 24 hr. [DOX][IR783]INMs were introduced at 0.89 μM.1 The results are represented as mean+SD (n=3), statistically significant p s 0.05 (*) was evaluated using the student t-test.

FIG. 64 is a graph showing cell viability of [DOX][ICG]INMs in MCF-7 cells in the presence of different endocytosis inhibitors pre-incubated for 2 hr at 37° C. prior to drug introduction for 24 hr. [DOX][ICG]INMs were introduced at 0.55 μM.1 The results are represented as mean+SD (n=3), statistically significant p 50.05 (*) was evaluated using the student t-test.

FIG. 65 is a graph showing In vivo toxicity after IT injection of single dose of 0.025 mg/Kg.

FIG. 66 are fluorescence emission images recorded after 24 hr of IT injection of drug at a) Ex/Em of 465/600 nm b) Ex/Em 780/845

FIG. 67 are fluorescence emission images recorded after 24 hr of IV injection of drug.

### DETAILED DESCRIPTION OF THE INVENTION

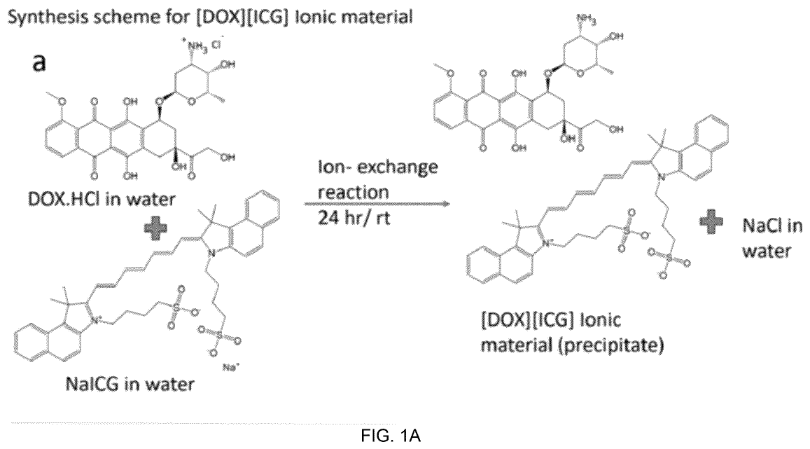

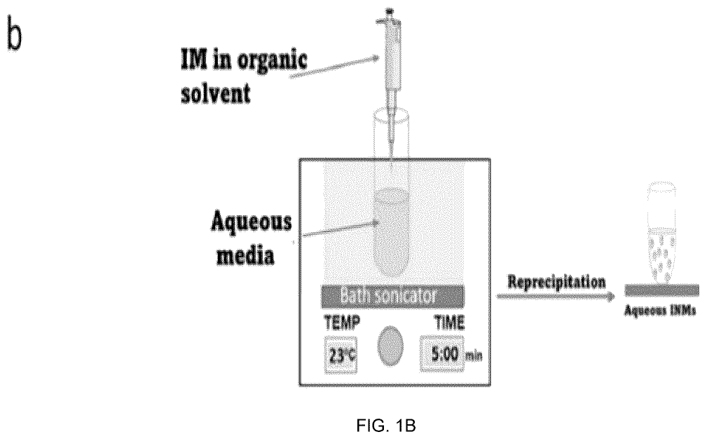

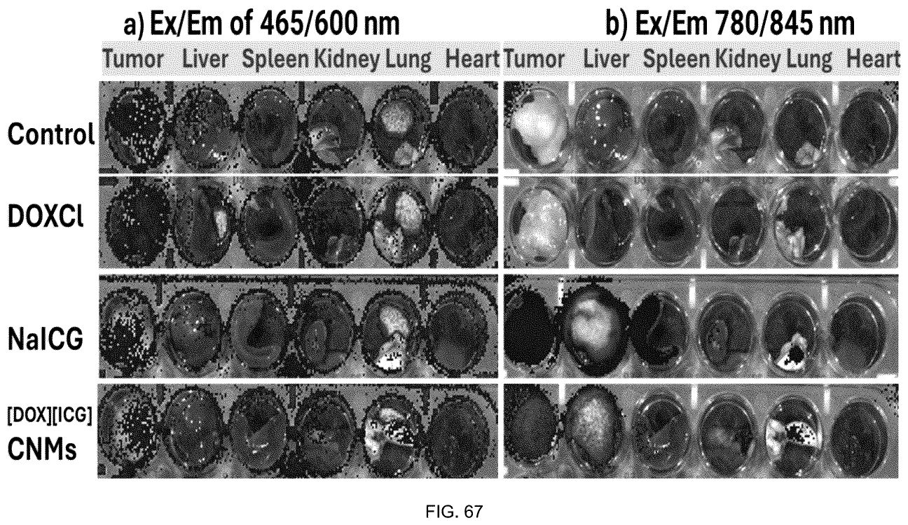

With reference to FIGS. 1A-67, the preferred embodiments of the present invention may be described. The present invention may be described with reference to three studies: Study 1, Study 2, and Study 3.

Study 1 EXPERIMENTAL

Chemicals: Doxorubicin hydrochloride (DOX.HCl), 2-[2-[2-Chloro-3-[2-[1,3-dihydro-3,3-dimethyl-1-(4-sulfobutyl)-2H-indol-2-ylidene]-ethylidene]-1-cyclohexene-1-yl]-ethenyl]-3,3-dimethyl-1-(4-sulfobutyl)-3H-indolium hydroxide inner salt, sodium salt (NaIR783, Lot #BCBZ9950), 2-[2-[2-chloro-3-[[1,3-dihydro-1,1-dimethyl-3-(4-sulfobutyl)-2H-benzo[e] indol-2-ylidene]-ethylidene]-1-cyclohexen-1-yl]-ethenyl]-1,1-dimethyl-3-(4-sulfobutyl)-1 H-benzo[e] indolium hydroxide inner salt, sodium salt (NaIR820, Lot #SHBM1333), 1.3-diphenylisobenzofuran (DPBF, Lot #STBD0599V) were purchased from Sigma-Aldrich (ST. Louis, MO). Indocyanine green (NaICG, Lot #KV5YO-LG) was purchased from Tokyo Chemical Industry (Tokyo, Japan). Pure lab Ultrapure water purification system was used to provide triply deionized water (18.2 M cm) (ELGA, Woodridge, IL). ACS grade dichloromethane (DCM) and ethanol were obtained from Thermo Fischer Scientific (Waltham, MA). 808 nm laser was purchased from Opto Engine LLC (Midvale, UT, US). MCF-7 breast cancerous cells were obtained from American Type Culture Collection (ATCC, Manassas, VA). Penicillin streptomycin and Trypsin-EDTA (0.25%) were both purchased from Caisson Lab (Smithfield, UT). MTT (3-(4,5-dimethylthiazol-2-yl)-2,5-diphenyltetrazolium bromide), 4′,6-diamidino-2-phenylindole (DAPI), Cell media (Dulbecco's Modified Eagles Medium (DMEM)), Phosphate buffer saline (PBS) pH 7.4 and dimethyl sulfoxide (DMSO) were all purchased from Sigma-Aldrich, (ST. Louis, MO). Fetal bovine serum (FBS) was obtained from Atlanta Biologicals (Lawrenceville, GA). YO-PRO (LOT #1597071) was purchased from Thermo Fisher Scientific and propidium iodide PI (Lot #21 P0211) was obtained from Biotium.

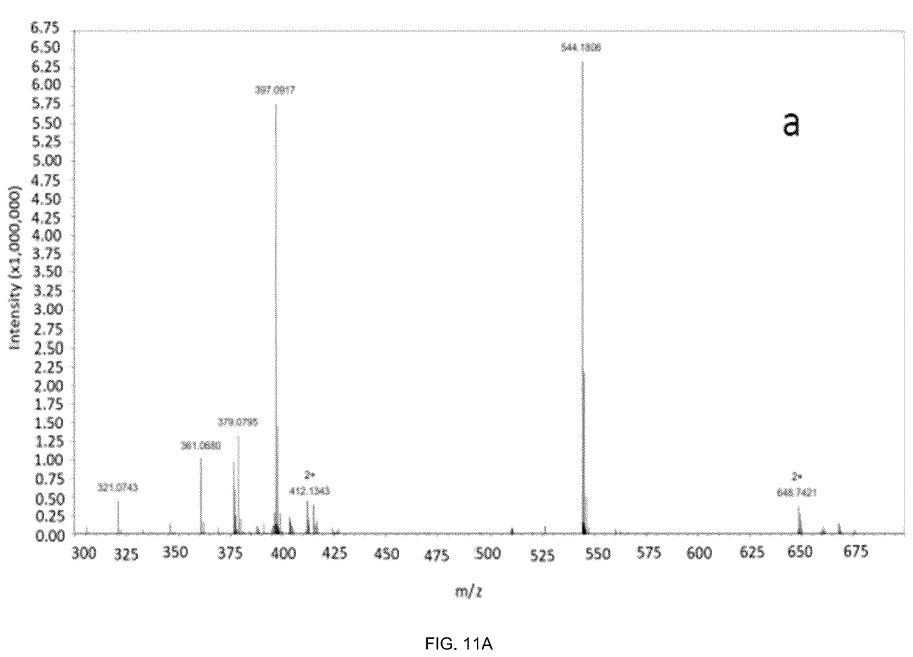

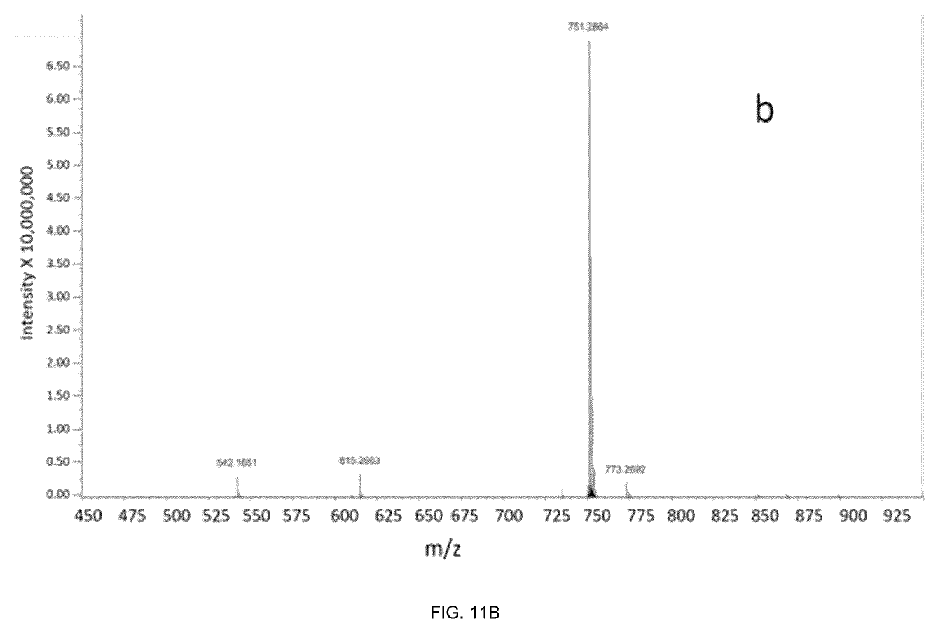

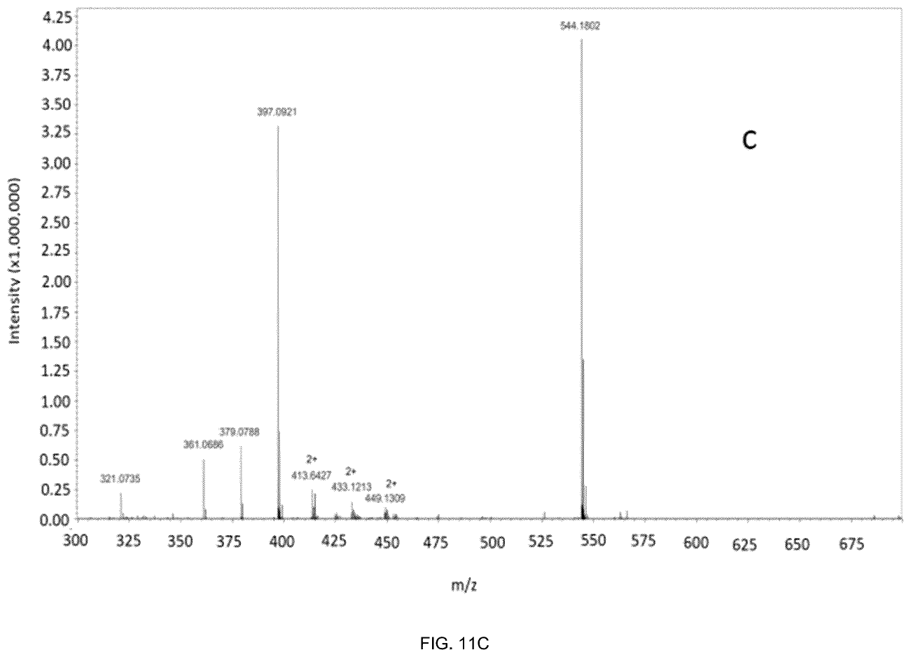

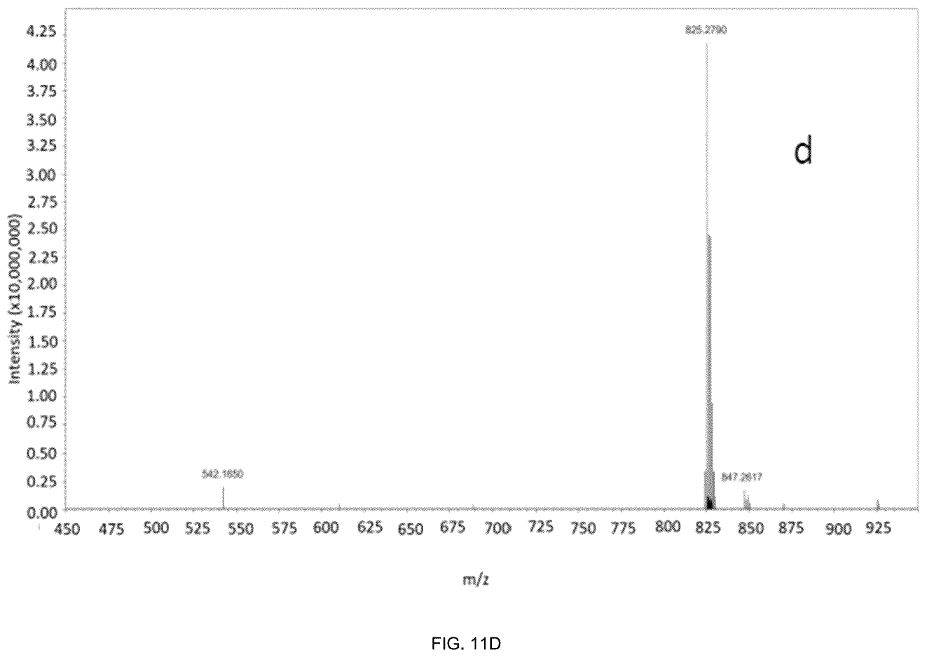

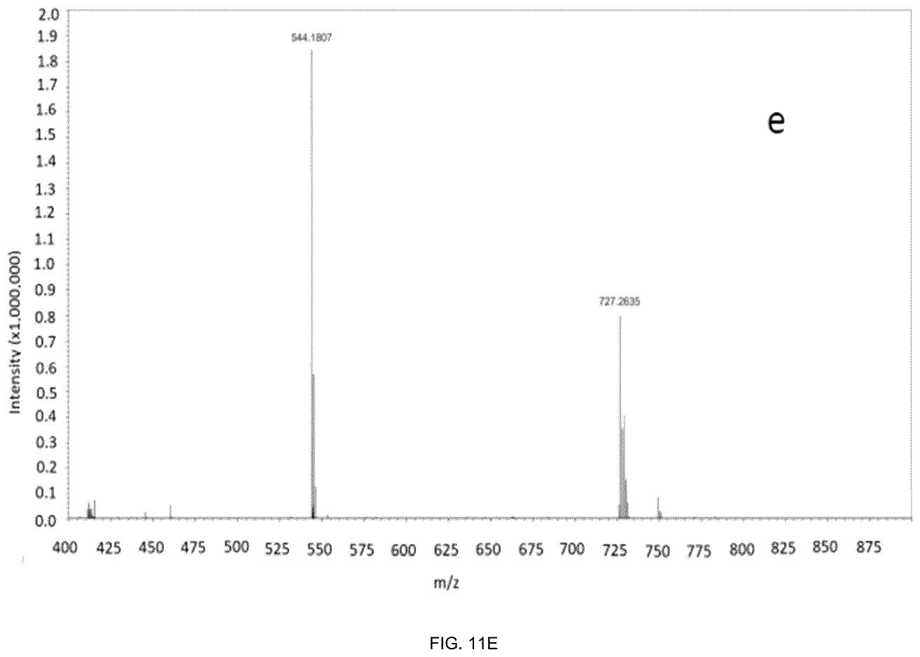

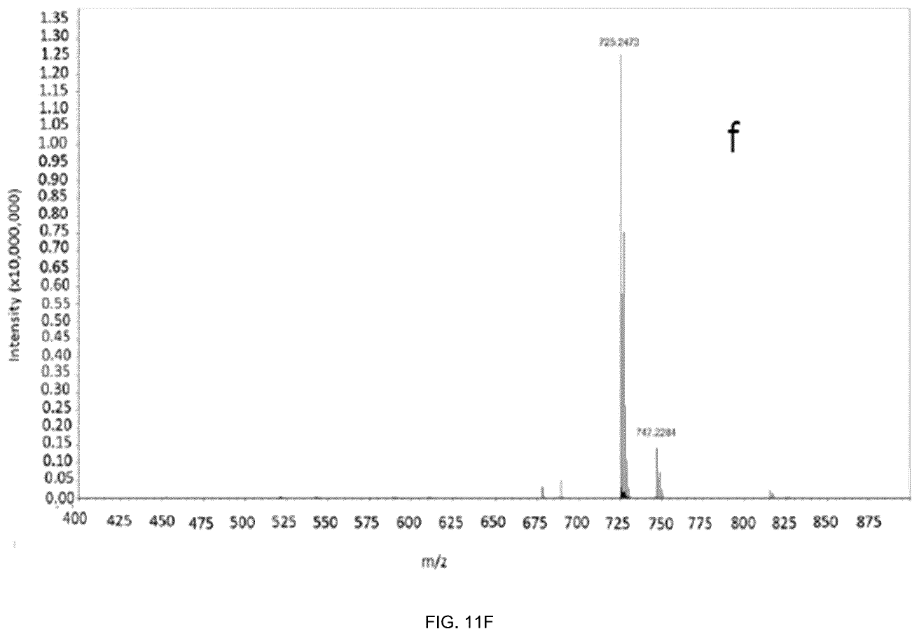

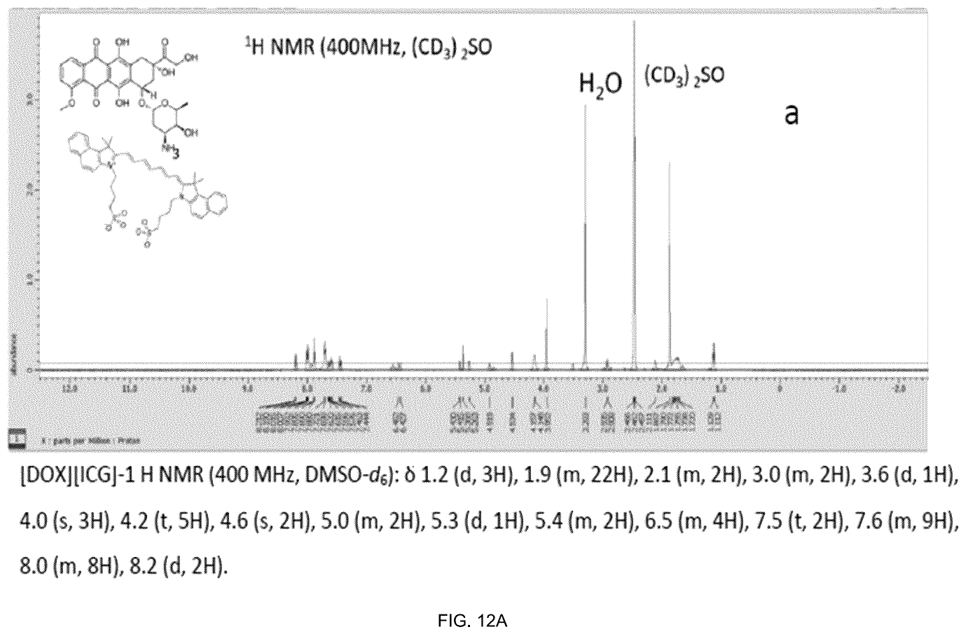

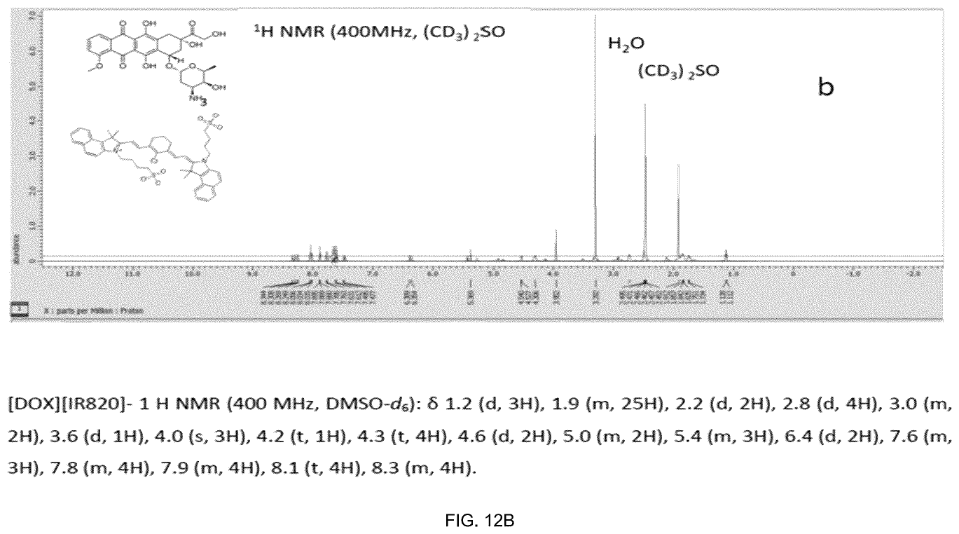

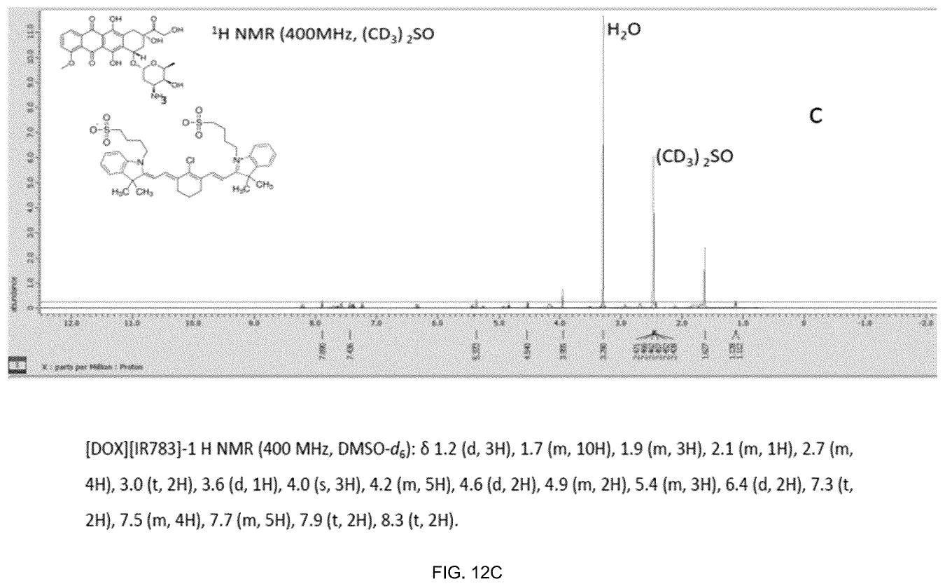

Characterization of Doxorubicin-based chemo combination drugs: Electrospray ionization mass spectrometry (ESI-MS) was used to characterize the chemo-PTT combination IMs utilizing a Bruker (Billerica, MA) Ultraflex 9.4T in methanol solvent. This technique was used to confirm the presence of both cation and anion in all three combination drugs with peaks corresponding to the mass to charge ratio in the positive and negative ion mode as shown in FIGS. 11A-11F. These chemo-PTT IMs were further characterized with JEOL 400 MHz nuclear magnetic resonance (NMR) instrument, using deuterated DMSO solvent as shown in FIGS. 12A-12C.

Characterization of INMs: The dynamic light scattering (DLS) method was utilized to estimate the hydrodynamic diameter as well as the polydispersity index of the INMs in DI water using a Zetasizer pro red Malvern Instrument (Malvern Panalytical Limited, Worcestershire, UK). Additionally, the size of the INMs were verified using a Transmission electron microscopy (TEM) with JOEL FEI Tecnai F20 200 KeV microscope.

Photophysical characterization: Absorption spectra of the parent compounds, the synthesized IMs and the INMs were recorded using a UV-visible spectrophotometer (Agilent Cary 5000, Santa Clara, CA) while their fluorescence emission spectra were recorded using a Fluorometer (Horiba FluoroMax, Kyoto, Japan). For absorbance measurement, a polished two-sided quartz cuvette with a 10 mm pathlength (starna cells) was used as against an identical control cell filled with the sample's solvent. Fluorescence measurements were performed using a four-sided starna quartz cuvette having 1 cm path length. An identical slit width, 0.1 second integration time, and right-angle geometry were used for all samples' measurements.

Fluorescence quantum yield measurement was performed for all NIR containing compounds using a relative method by employing NaICG as the standard with reported fluorescence quantum yield value of 0.14 in ethanol.

Fluorescence lifetime measurement: A Horiba fluorometer featuring a Delta Hub controller and time correlated single photon counting (TCSPC) was used to measure the fluorescence lifetime for the combination IMs in ethanol. A 455 nm wavelength NanoLED served as the excitation source for measurement. Using exponential fitting of the raw data, the lifetime data was examined using DAS6 software.

Light to heat conversion efficiency: Investigation of the photothermal effect of any drug requires subjecting the PTA to laser irradiation and monitoring the change in temperature over time. The heat efficiency of the NIR parent dyes and Chemo-PTT combination INMs was investigated to determine the drug's PTT potency. To examine the PTT effects of the various NIR parent compounds, the drugs were prepared in different solvents such as pure water, PBS and cell media containing FBS. Similar concentration of each INMs was prepared in various media via reprecipitation method as previously stated and was left to stabilize for 20 mins prior to experiment. In a typical experiment, 1 mL of 50 μM solution of the various samples (NIR parent dyes and INMs) was subjected to a 5 mins laser treatment (808 nm, 1 Wcm⁻²). The temperature of the entire solution was measured using a thermocouple probe for every 30 secs. The sample was irradiated with 808 nm laser light for the first 5 mins, and the next 5 mins the sample's cooling temperature was recorded after the laser was turned off.

Singlet oxygen quantum yield (SOQY): Photosensitizers (PS) capable of absorbing light upon irradiation can get promoted to the excited triplet state from excited singlet state via a non-radiative pathway known as intersystem crossing. At the excited triplet sate, the PS reacts with molecular oxygen present to produce ROS. Literature have shown that NIR-absorbing dyes such as NaICG, NalR783, and NalR820 can produce ROS upon absorption of light and is dependent on the media. The SOQY efficiencies of NIR dyes are fairly low in water (around 0.8 percent) but it's very high (around 20%) in other media.

To ascertain the PDT effect by the NIR dyes in comparison to the newly synthesized combination drugs, SOQY experiment was conducted. The experiment was performed using parent dyes, combination IMs and INMs. It is very important to understand the role of chemotherapeutic cation (DOX) on the ROS production of NIR dyes in the combination IMs and nanomedicines. Therefore, a parent NIR dye solution, combination IM, or INMs was mixed independently with DPBF probe solution in ethanol for SOQY experiment. In a typical experiment, DPBF and parent NIR drug were dissolved in ethanol, separately. A concentration of 5 μM drug in 100 μM DPBF was achieved by mixing an equal volume of DPBF and drug solution. By measuring the decrease in absorbance at 411 nm after exposing the solution mixture to 808 nm laser (1 Wcm⁻²) for 15 secs at a time for a total of 60 secs, the rate of ROS formation was calculated. A UV/Vis spectrophotometer was used to track the decrease in absorbance of DPBF at 411 nm after each 15 secs exposure. The parent NIR dyes and the combination drug's ROS quantum yields in ethanol were calculated using NaIR820 as a reference.

For SOQY experiment involving the INMs, only IMs and DPBF were prepared in DMSO separately and then nanoparticles were prepared via reprecipitation method. To prepare the INMs, a stock solution of IMs or DPBF was initially prepared in DMSO. INMs were prepared by adding an aliquot of IM dropwise to the vial containing water in an active sonication bath left to sonicate for 5 mins and proceeded with a 20 mins stabilization prior to any measurement. The parent NIR soluble dyes were prepared directly in water. The final concentration of the resultant solution (parent NIR dyes or INMs) used was 10 μM drug in 25 μM DPBF. A similar irradiation protocol was followed, and formation of singlet oxygen was detected by measuring the decrease in DPBF absorbance at 411 nm. This was quantified using Equation 1. A control experiment in which a solution of DPBF in ethanol was irradiated under identical condition was designed to verify that the decrease in absorbance at 411 nm is caused by the drug.

$$ϕ Δ ( x ) = ϕ Δ ( s t d ) * \frac{Sx}{S s t d} \qquad ( 1 )$$

where φ^{Δ(std)} denotes the ROS quantum yield of standard. Sₓ and S_{std} are the slope obtained from the absorbance vs time graph showing the decrease in DPBF absorbance at 411 nm for unknown and standard sample respectively. By comparing the absorbance decrease of the probe in the presence of combination INMs, NaIR783 was employed as the standard to compute the SOQY according to Equation 1. The ROS quantum yield of Chemo-PTT INMs and IMs was calculated using references with reported literature values of 0.7% in water and 7.7% in ethanol for NaIR783 and NaIR820 respectively.

Photostability Measurements: Photostability of the various free NIR dyes and chemo-PTT combination drug was investigated by kinetically measuring the fluorescence emission intensity over a 30 mins period at intervals of 0.1 seconds. A quartz cuvette with a four-sided polished window and a 1 cm pathlength was used for the measurements. Briefly, 2 μM solution of the parent compound and chemo-PTT combination drug was prepared from ethanol stock, and the photostability measurement was performed using the absorbance wavelength maxima as the excitation wavelength and recording the fluorescence emission at respective fluorescence emission maxima wavelength oof compounds. The excitation and emission slit widths were both adjusted to 14/14 nm.

Cellular Uptake: The detailed characterization of drug is performed in vitro to investigate their therapeutic potential. For the test, only MCF-7 cells were used for all in vitro experiments. The potential of these drugs in other cell lines as well as in normal cell lines will be presented in another paper. Since combination drugs are being used in a nanoparticle form as compared to their soluble parent chemo and PTT drugs, it may impact the cellular uptake and consequently affect the cytotoxicity. Therefore, cellular uptake tests were carried out to ascertain the concentration of drugs internalized by MCF-7 cells over a specific period. In a typical experiment, 6×10⁵ cells were seeded and left to incubate for 24 hrs in a six well plate. A 20 μM concentration of parent NIR dyes, soluble chemo drugs or INMs were introduced to each well with a total volume of 3 mL in each well and incubated for 6 hrs. Uninternalized drugs were eliminated after the stipulated period, and cells were carefully washed repeatedly thrice with PBS. Cells were further broken up by addition of 3 mL DMSO, allowing the internalized drug to be quantified by a UV-Vis absorbance spectrophotometer.

Cell Culture/Cell Viability studies: For in vitro experiments, monolayer of MCF-7 cell lines were maintained in complete media at 37° C. and 5% CO₂. MCF-7 cells were grown in DMEM supplemented with FBS (10% v/v) and antibiotic solution containing penicillin/streptomycin (500 units/mL). When cells reached the desired confluency, they were trypsinized to subculture, and the detached cells were counted using a hemocytometer after being stained with trypan blue exclusion dye. For a 24 hrs dark cytotoxicity experiment, 10⁴ cells per well were plated in 96 well plates and incubated at 37° C. and 5% CO₂. After that, cells were exposed to various INMs concentration for 24 hrs. Different concentrations of INMs were prepared by reprecipitating stock solution in DMEM media under sonication, while maintaining a sterile environment. To prevent any cellular toxicity from DMSO, the amount of DMSO utilized was limited to a maximum of 0.5%. For each experiment, complete media controls and DMSO controls were taken into consideration. PBS was used to wash the cells before the treatment. MTT assay was used to determine cell viability. The optical density of MTT-formazan at 570 nm was measured using a microplate reader (Biotek Synergy H1, Winooski, VT). For in vitro experiments, each experiment was carried out in triplicate and repeated thrice. Except as otherwise stated, all data with error bars are provided as mean+standard deviation (SD). The two-tailed student “t” test was used to evaluate the significant difference in the mean values. Values that differ significantly are shown by the symbols *p<0.05, **p<0.01, and ***p<0.005.

Light cytotoxicity experiments were performed to ascertain PTT efficiency of the parent NIR drugs as well as the PTA present with DOX in the combination INMs. Cells were treated in a similar manner as described above. In a typical experiment, 10⁴ cells were seeded in a 96 well plate in alternate wells and incubated for 24 hrs. Cells were further treated with INMs or parent dyes and incubated for 6 hrs to minimize the cytotoxic effect from DOX while trying to elucidate mostly the PTT effect. After 6 hrs of drug incubation, the media containing drug was aspirated and the cells were washed twice with PBS to remove the external uninternalized drugs. Cell media was replaced in each well and each well plate was exposed for 5 mins to a near infrared laser (808 nm, 1 Wcm⁻²). After 24 hrs incubation, MTT assay was used to investigate the cytotoxicity of the NIR dyes. A control experiment without any PTA containing drug was subjected to light and conducted under similar conditions.

Cell death mechanism: Cell death mechanism was investigated using caspase assay and flow cytometry. For caspase 3/7 staining, cells were plated at a density of 2×10⁵ per well in a 24 well plate with pre-introduced coverslips and were incubated for 24 hrs. Cells were treated with 5 μM INMs prepared in cell media after being washed with PBS and then followed by incubation for 6 hrs. After the stipulated time, the uninternalized drugs were aspirated and the cells were treated with 5 μM of caspase 3/7 assay for 60 mins. Fixation of the cells was done with 4% paraformaldehyde for 15 mins. Cells were permeabilized for 30 min using blocking solution (0.3 M glycine, 10% BSA, and 1% saponin in PBS). Cells were further washed with PBS prior to the addition of DAPI (nuclear stain) at 300 nM concentration. Cells were washed with PBS before mounting on the glass slide and imaged using the laser scanning confocal microscope (Zeiss, LSM 800).

For flow cytometry using PI (propidium iodide)/YO-PRO staining, cells were plated at approximately 1×10⁶ cells in a 6 well plate and incubated overnight. Cells were washed with PBS and incubated with 5 μM INMs for 6 or 48 hrs. After drug treatment, both floating and adherent cells were harvested by addition of 0.5 mL trypsin and monitored closely for cells complete detachment under the microscope. Trypsin was neutralized with 1 mL serum containing media and the cell mixture was transferred into a centrifuge tube proceeded with spinning at 1100 RPM for 5 mins. The cell pellet was washed with cold PBS and re-centrifuged again for 5 mins. Cells were resuspended in a 0.25-0.5 mL PBS in flow cytometry tubes, followed by addition of 1 μL of PI and YO-PRO solutions to the cell mixture according to manufacturer's protocol. The tubes were all placed in an ice-bath and proceeded for flow cytometry data acquisition.

Statistics: The two-tailed Student's t-test was used for statistical analysis. Statistical significance was defined as p-values of *p<0.05, **p<0.01, and ***p<0.005. The mean+S.D. is used to express the results, which are representative of at least three studies.

### Results and Discussion:

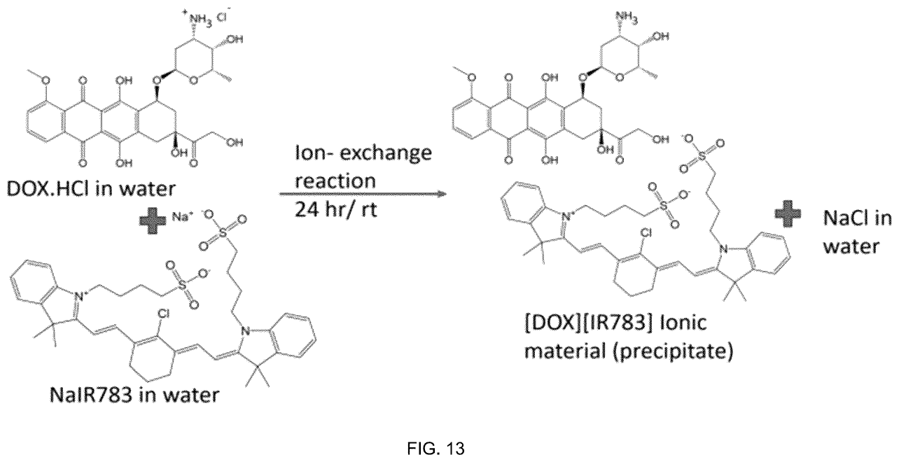

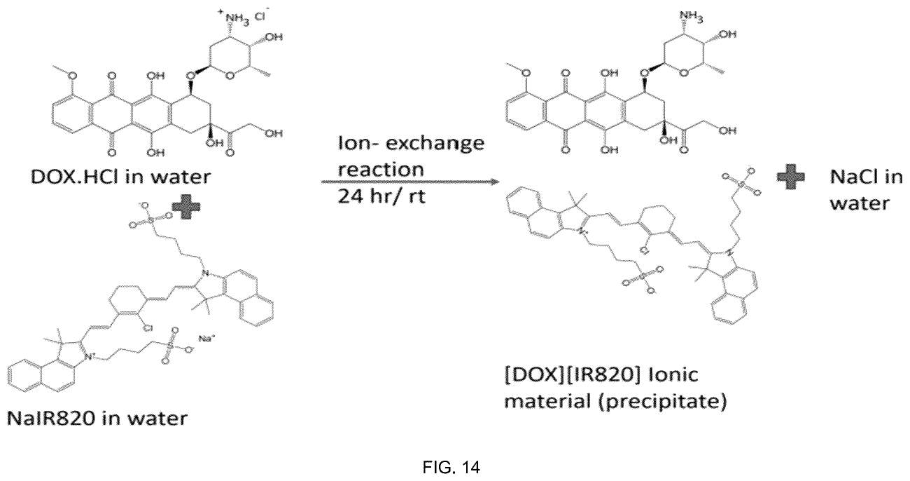

Synthesis and characterization of DOX-based chemo combination drugs (IMs): Three chemo-PTT combination drugs were synthesized from two ionic parent compounds using a simplistic, rapid, economical, one step ion exchange method as shown in FIG. 1A. For synthesis of [DOX][ICG], equivalent mole ratio of DOX and NaICG were separately dissolved in water. Aqueous solutions of both DOX and NaICG were combined, and precipitate of the [DOX][ICG]combination IMs were observed immediately. To make sure the ion exchange reaction was complete, the mixture was agitated continuously for 24 hrs. The resultant mixture was centrifuged at 3800 RPM for 5 min, and the supernatant was removed to recover precipitate of IMs. The precipitate was washed three times with water to remove the by-product (NaCl). The resultant drug [DOX][ICG]was lyophilized to remove moisture impurities from IMs before use for further studies. Since NaIR783 and NaIR820 are also hydrophilic, similar protocol was followed to synthesize the [DOX][IR783] and [DOX][IR820]chemo-PTT combination drugs respectively as shown in FIGS. 13-14.

All IMs were characterized in detail to investigate the presence of cation and anion in the combination drugs as well as the purity of the compound. Synthesized IMs, [DOX][ICG], [DOX][IR820] and [DOX][IR783]were characterized using ESI-MS. The presence of both the cation and anion was verified by the mass-to-charge ratio peaks in the positive and negative ion modes. Mass spectra data for all compounds are shown in FIGS. 11A-11F. NMR spectroscopy was also used to further characterize the IMs. NMR spectra and results are presented in FIGS. 12A-12C.

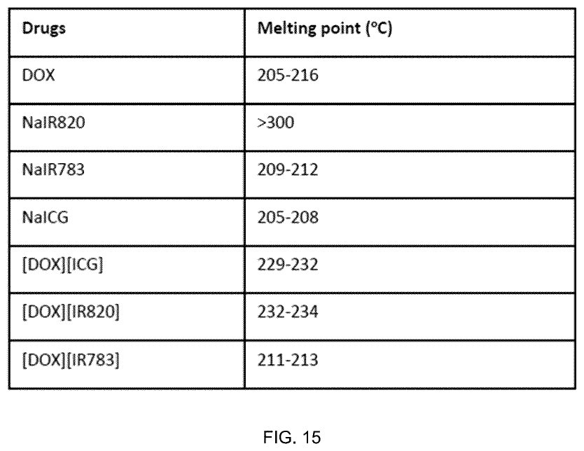

After confirming the purity of the materials, the compounds were further characterized to investigate their physicochemical properties. The melting points of [DOX][ICG], [DOX][IR820] and [DOX][IR783]INMs were 230° C., 233° C. and 212° C. respectively. FIG. 15 shows the melting point range for all the compounds compared with their parent NIR dyes. The melting points of all combination drugs are under 250° C.

Synthesis and characterization of INMs: INMs were prepared by reprecipitation method in water or cell media from the IMs as shown in FIG. 1B. These are carrier free nanoparticles which were prepared in pure water/cell media under sonication. In a nutshell, 1 mM concentration of the chemo-PTT combination IMs were initially prepared in ethanol or dimethyl sulfoxide (DMSO). To a scintillation vial containing deionized water or cell media in an active sonication bath, a little amount of the stock solution was introduced dropwise. The sample (INMs) was sonicated for 5 mins and stabilized for 20 mins before performing any experiments. The shape and size of nanoparticles were characterized in detail.

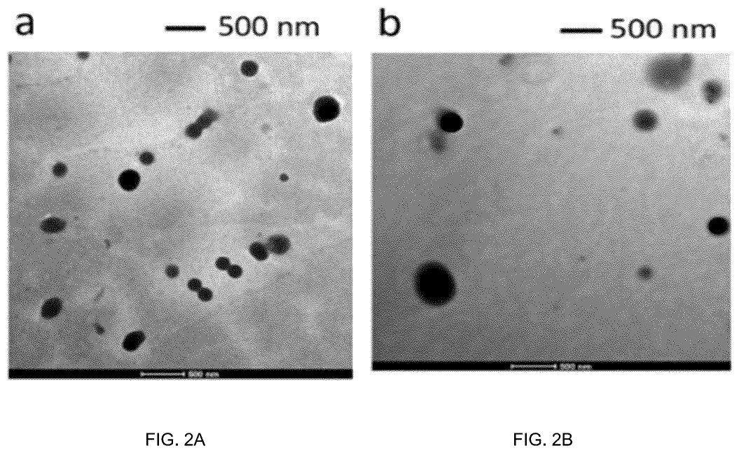

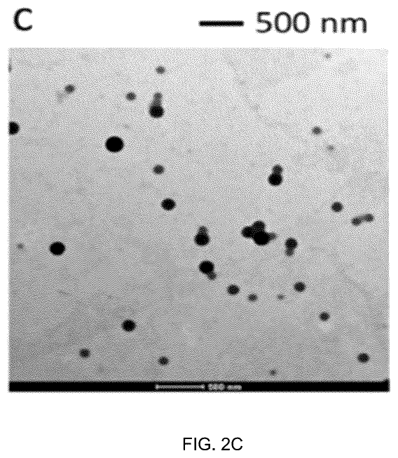

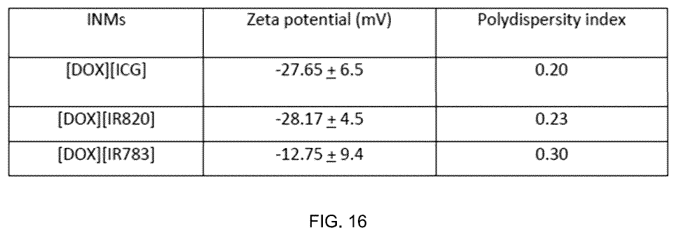

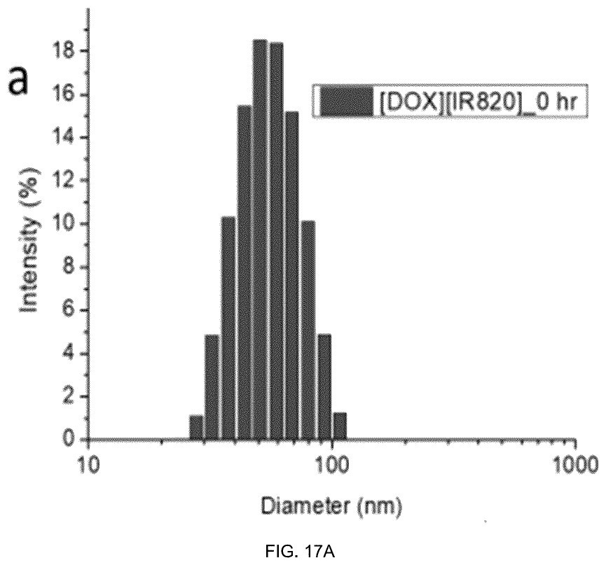

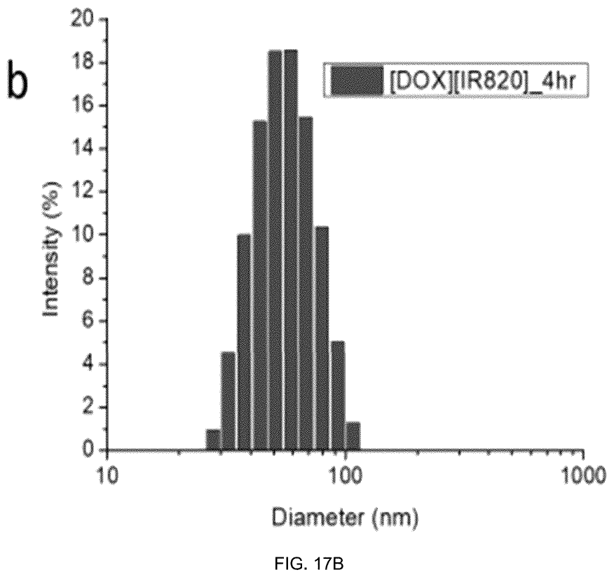

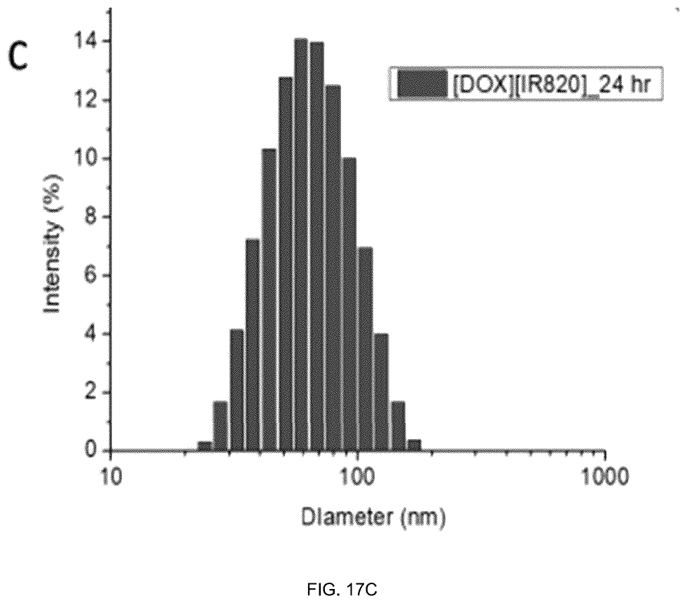

INMs physical properties were investigated in detail using different techniques. The shape and size of the INMs were determined using Transmission electron microscopy (TEM). The INMs were found to be spherical in shape as shown in FIG. 2A-2C. The observed sizes for [DOX][ICG], [DOX][IR783] and [DOX][IR820]were found to be 135±12.5 nm, 174±22.5 nm, and 105±15.9 nm respectively. NPs with diameters between 10 and 200 nm have been reported to actively target malignant tissues due to the EPR effects. Zeta potential measurements were performed using Zetasizer pro red Malvern equipment. The zeta potential results reported in FIG. 16 showed that all nanoparticles exhibited very low negative surface charge which suggest that the NIR dyes are mainly residing at the surface of the nanomaterials. All three INMs also exhibit polydispersity index less than 0.30 (FIG. 16) which indicates a homogeneous population of the nanodrug. [DOX][ICG]exhibited the lowest PDI. Whereas [DOX][IR783]depicted the highest PDI which is attributed to its lowest zeta potential value. Dynamic light scattering (DLS) experiments were performed at various intervals to test the stability of the nanoparticles. Examination of data revealed that the [DOX][IR820]INMS were stable for 24 hrs as shown in FIGS. 17A-17C.

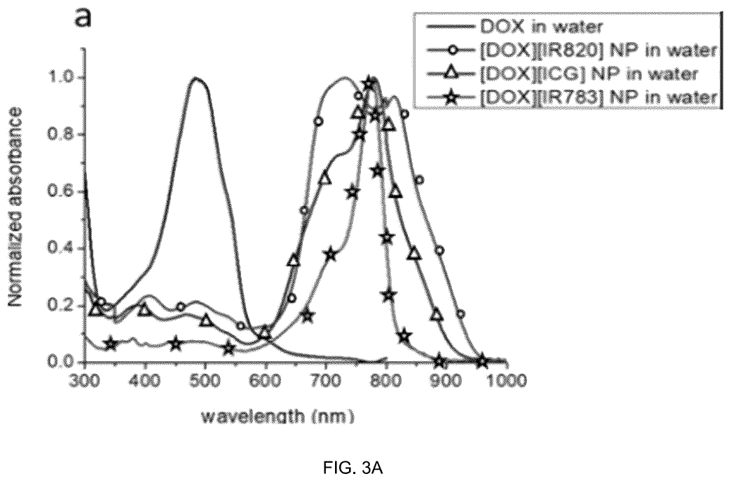

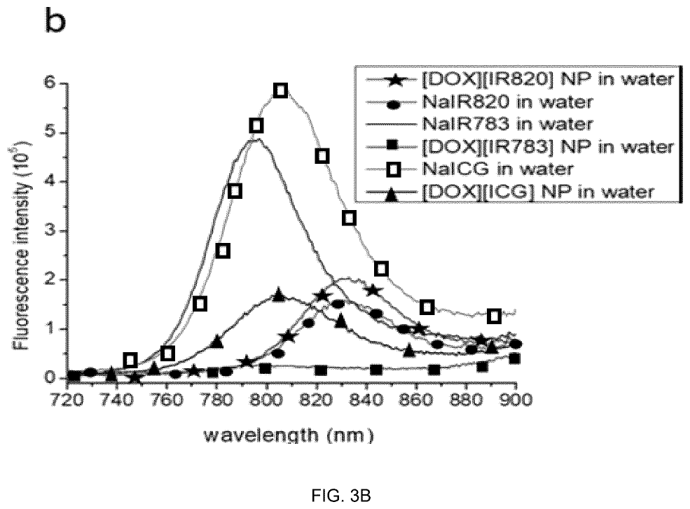

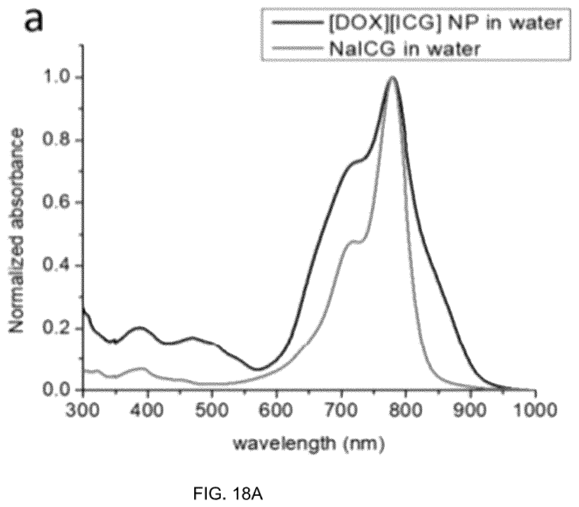

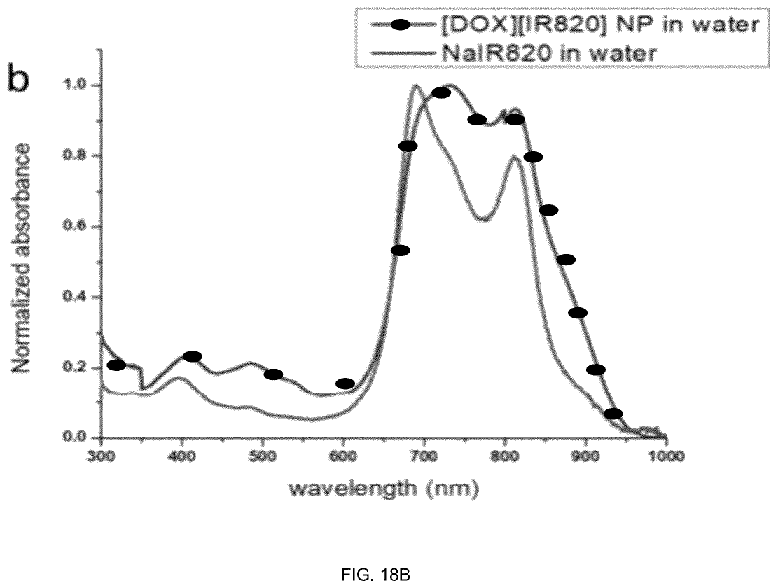

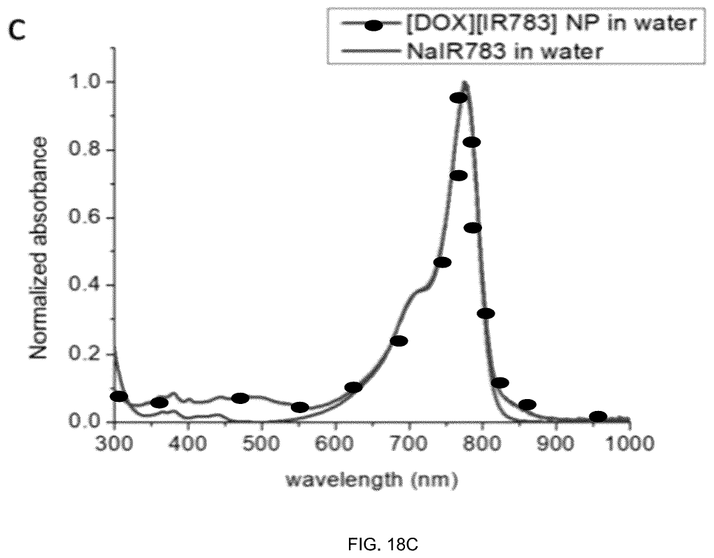

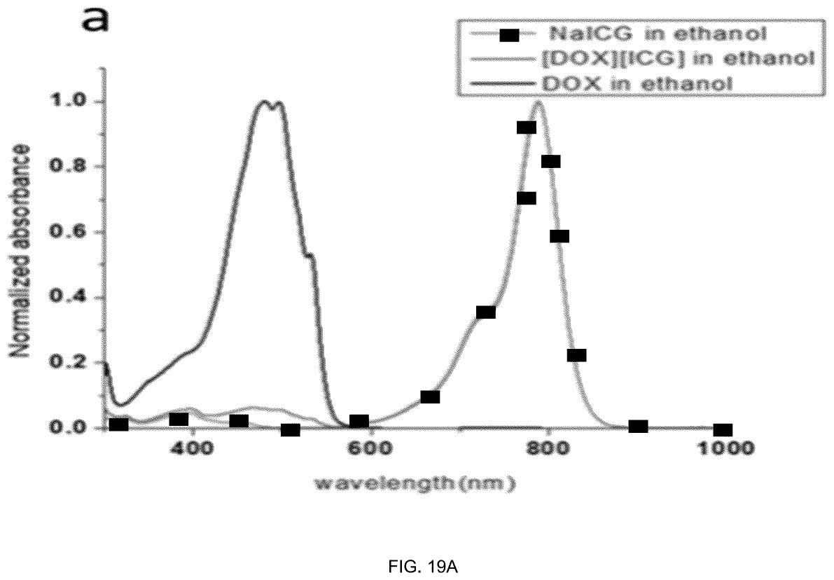

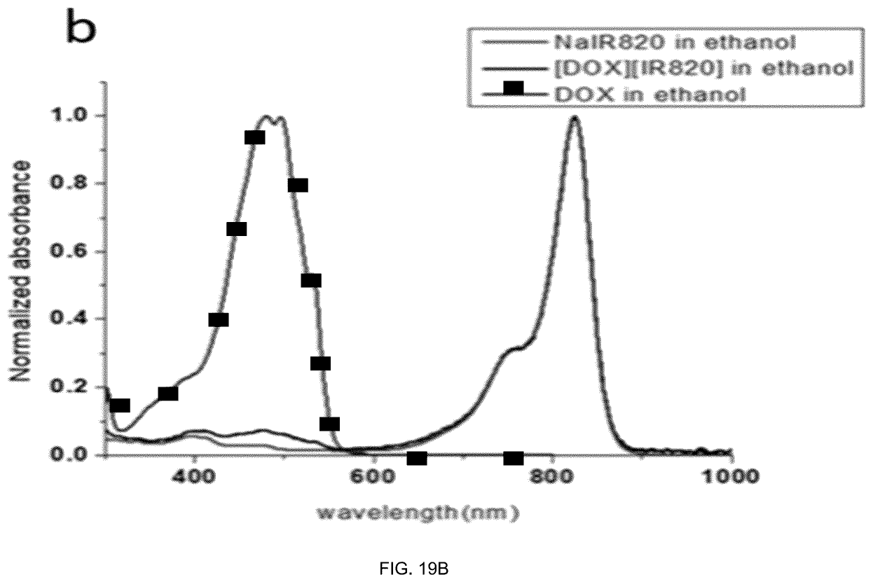

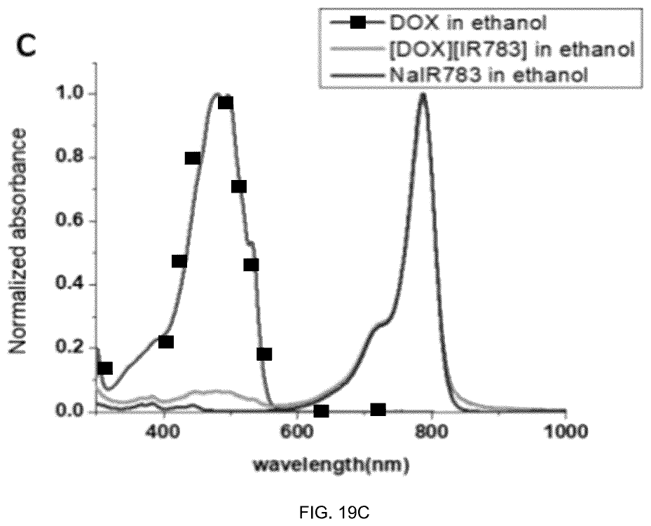

Photophysical characterization: The photothermal efficiency of a PTA mainly relies on its photophysical properties. The photophysical characteristics of parent NIR dye compounds, combination IMs and INMs were investigated in water and ethanol as shown in FIG. 3, FIGS. 18A-18C, and FIGS. 19A-19C The absorption maxima wavelength, molar absorptivity, fluorescence emission and quantum yield were determined since all these key photophysical parameters can influence the phototherapeutic activities such as reactive oxygen species (ROS) quantum yield and light to heat conversion efficiency of the PTA in the presence of DOX cation in the combination IMs and INMs. Hence, the changes in the absorption and fluorescence spectra of the combination drug in water and ethanol were investigated and compared with their respective parent compounds to determine the effect of counterion.

Absorption spectra results confirmed the presence of both cation (DOX) and anion (NIR dye) in the IMs and INMs. Examination of absorption spectra of ICG compounds revealed that the ICG anion absorption peak exhibited significant broadness when NaICG were converted into combination INMs. In water, both NaICG free dye and [DOX][ICG]INM displayed a similar absorbance peak at 777 nm with a shoulder peak around 720 nm (FIG. 18A). However, a slight bathochromic shift occurs for both compounds in ethanol in comparison to water from 777 nm to 789 nm as shown in FIG. 18A and FIG. 19A. Whereas no significant change in the shoulder peak (around 720 nm) was observed for both NaICG and [DOX][ICG] in ethanol.

NalR820 free dye and its INM, [DOX][IR820]showed two distinct absorbance peaks in water for IR820 anion (FIGS. 3A-3B; FIG. 18B). Absorbance peaks for NaIR820 were observed at 690 nm and 813 nm. A bathochromic shift (red shift) was observed in the first peak from 690 nm to 733 nm (broad) while the peak at 813 nm remained unchanged upon conversion of NaIR820 into [DOX][IR820]INMs (FIG. 18B). Moreover, a very broad absorption peak for IR820 anions was observed in INMs that exhibited significant absorption at longer wavelength, which is highly desirable due to deeper penetration of longer wavelength radiation for the treatment of deeply seated tumor. In addition, both NaIR820 and [DOX][IR820]IM in ethanol depicted an absorbance maximum at 830 nm as well as a shoulder peak at 750 nm for IR820 anion (FIG. 19B) which is supported by literature.

The absorption spectra of other compounds based on IR783 were also recorded to determine any significant changes in the photophysical properties. The peak shape for IR783 anions did not change significantly when converted from NaIR783 to [DOX][IR783]INMs. For parent compound NaIR783 and [DOX][IR783]INM in water, both showed an absorbance peak maximum of 774 nm with a shoulder peak at 720 nm for IR783 anion (FIG. 18C). A slight red shift was observed for both compounds in ethanol with an absorbance maximum of 787 nm (FIG. 19C) for IR783 anion. No significant changes in the DOX peak were observed.

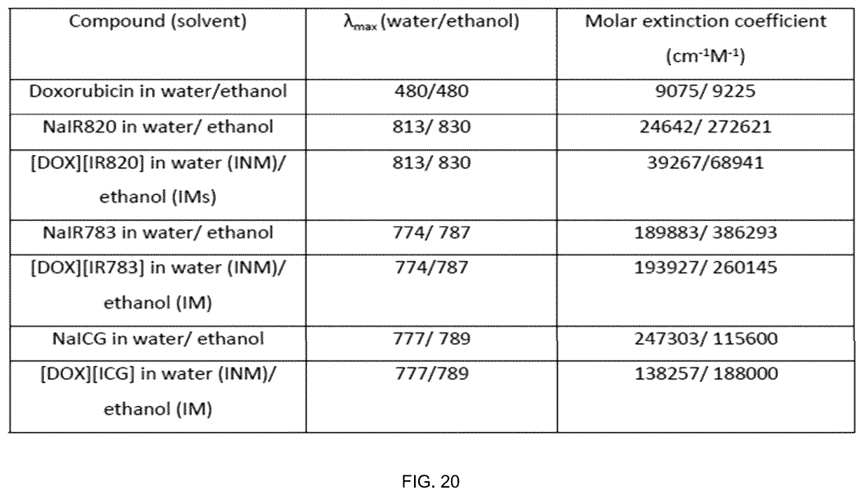

As anticipated, a higher molar absorptivity value was observed for the soluble combination IMs than in nanoparticle form (INMs) except ICG as reported in FIG. 20. In contrast, only NaICG shows greater molar absorptivity value in water than in ethanol. Thus, the absorption characteristics of a photosensitizer depends on the counter cation. Herein, the DOX counter cation enhanced the absorptivity characteristics of the NIR dyes.

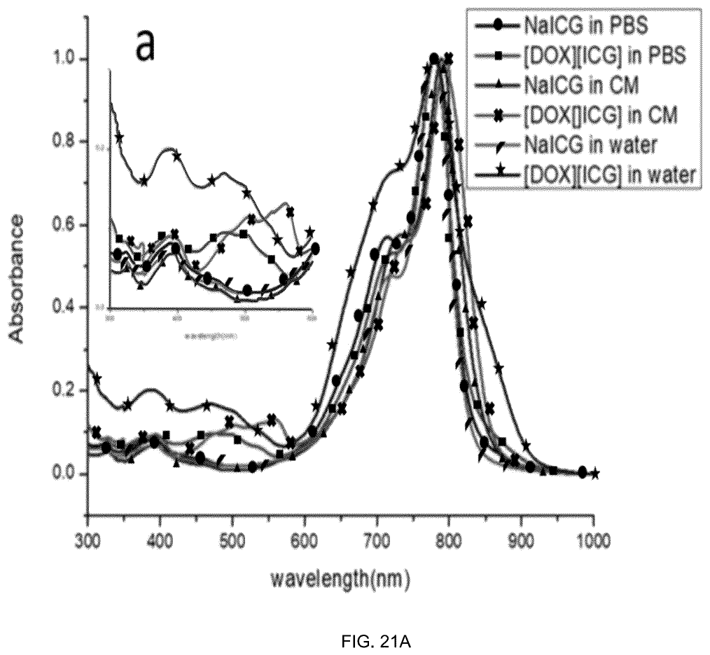

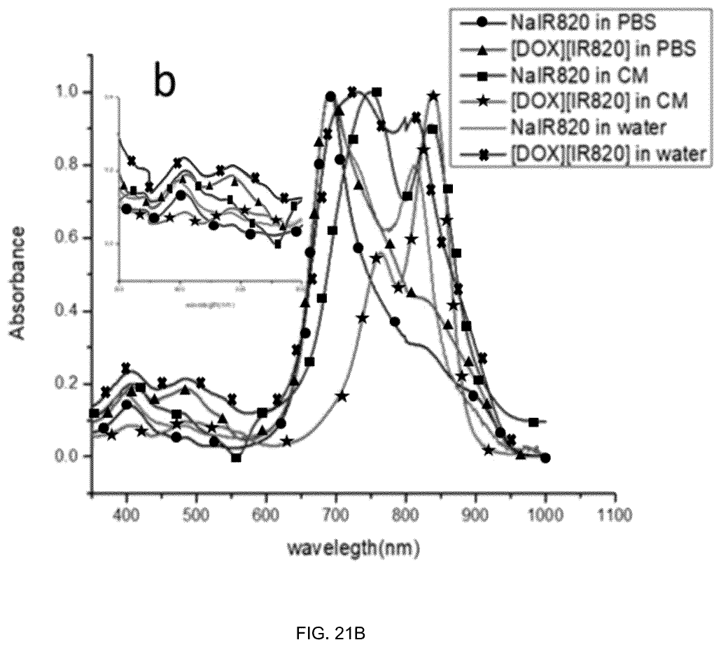

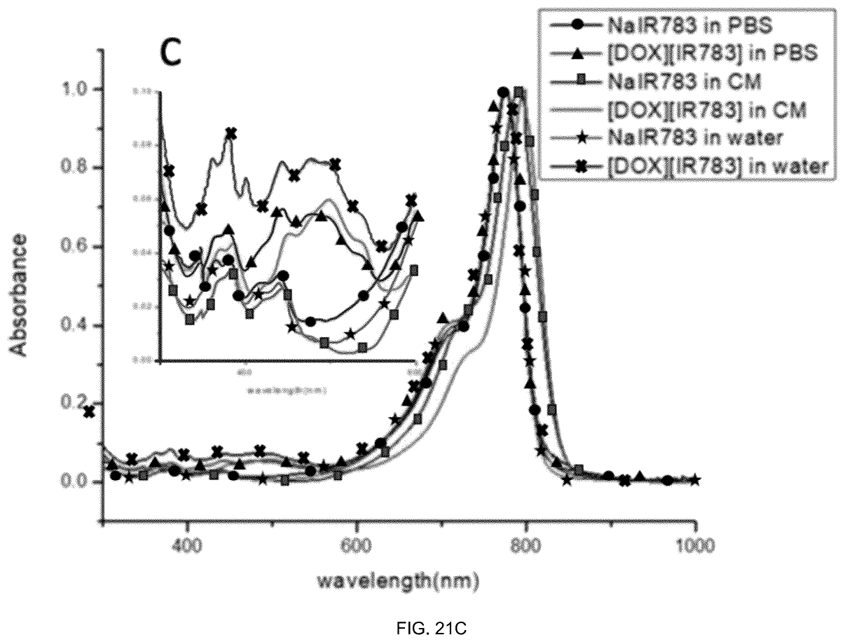

The absorbance of the samples was also recorded in PBS and cell media to mimic in vitro environment. Absorption spectra recorded in cell media and PBS are presented in FIGS. 21A-21C. Both ICG and IR783 based compound depicted similar peaks. However, significant changes in the peak maxima were observed for IR820 based compounds as shown in FIGS. 21A-21C.

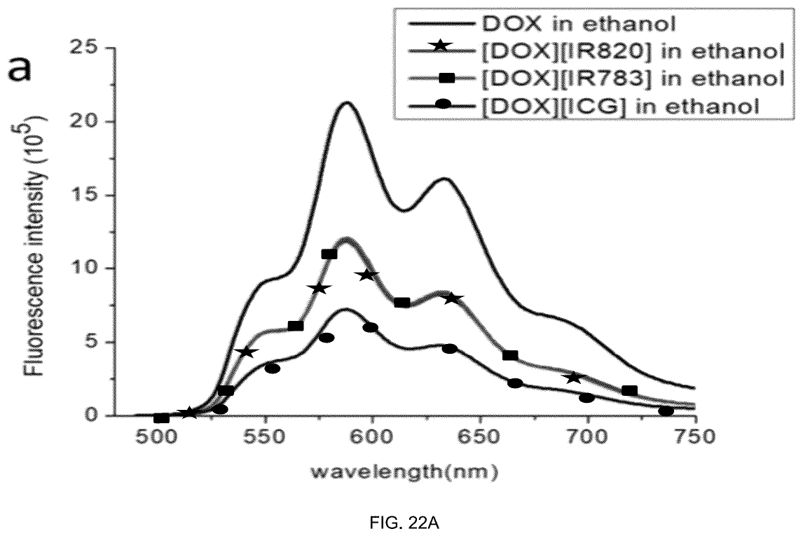

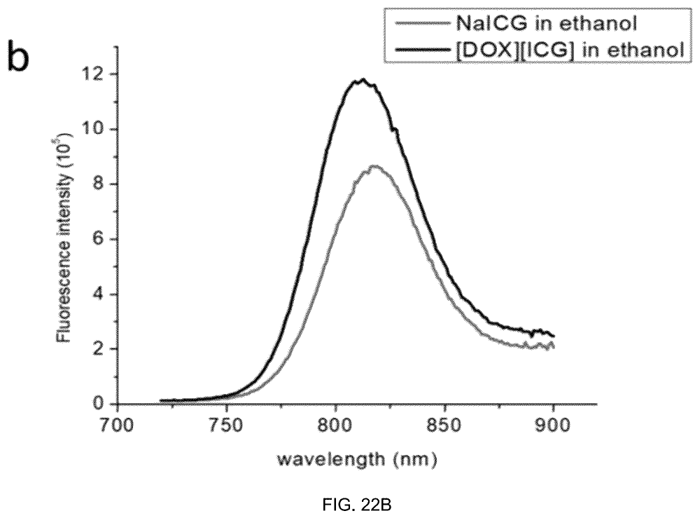

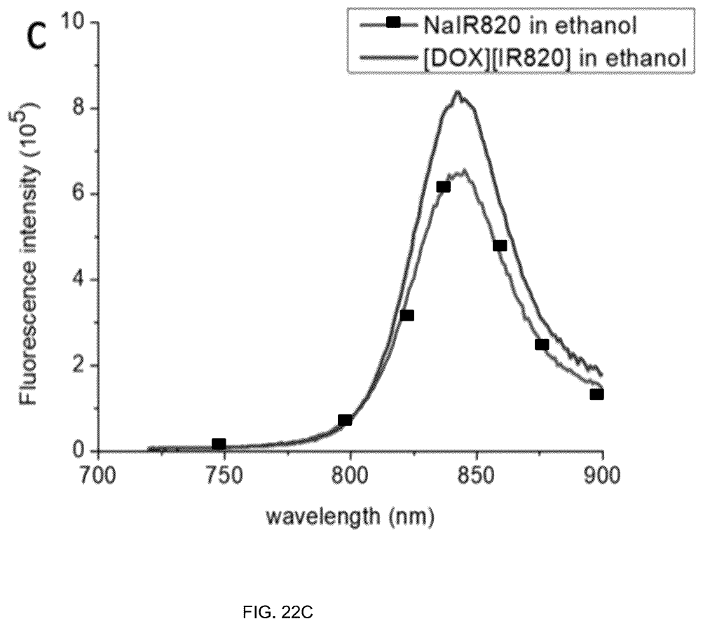

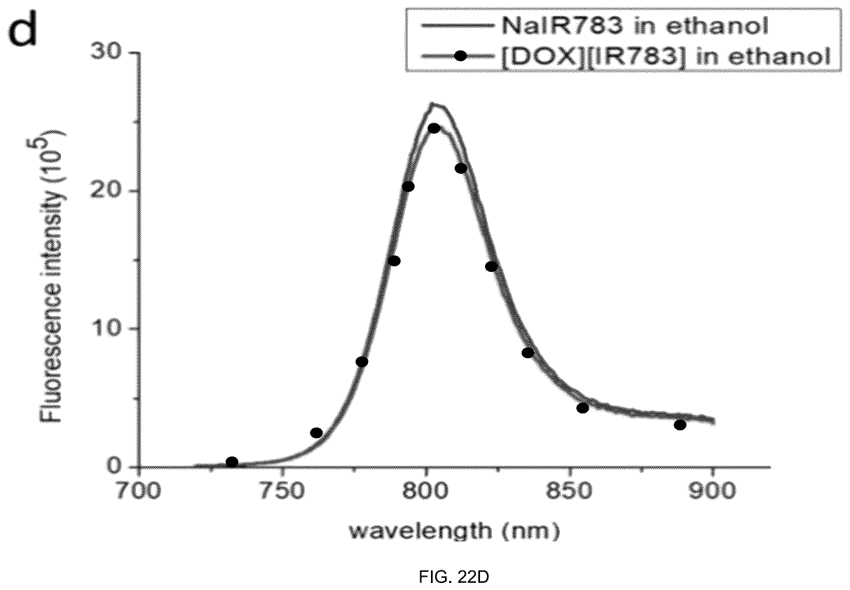

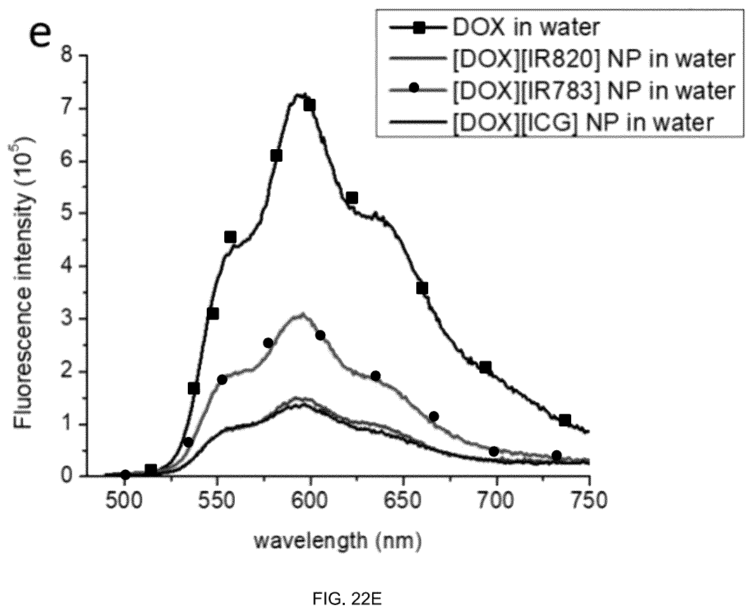

The detailed study of absorption spectra revealed some substantial alterations in the dye's photophysical characteristics. To investigate any changes in the fluorescence emission spectra of the compound, fluorescence measurements are performed. The fluorescence spectra of the IMs (FIGS. 22A-22D) and INMs as compared with parent drugs are shown in (FIG. 22E and FIG. 3B). When the INMs were excited at the absorbance peak maxima of DOX (480 nm), a similar shape of fluorescence emission spectrum was observed for DOX cation with a wavelength maximum at 595 nm along with two shoulder peaks at 555 nm and 639 nm in all three combination nanomedicines (FIG. 22E). Interestingly, the fluorescence emission intensity for DOX cation in each combination INMs was decreased significantly as compared to the parent DOX compound. The decrease in fluorescence intensity of [DOX][IR820] and [DOX][ICG] is very similar, while a significant decrease in fluorescence intensity was observed in the [DOX][IR783]INM. This decrease in fluorescence intensity signal for the DOX in all three INMs possibly signifies the existence of FRET mechanism since there is an overlap between DOX fluorescence emission spectra and NIR dye absorption spectra.

Fluorescence emission spectra for all chemo-PTT materials were also recoded at the excitation wavelength of the NIR dye. The fluorescence emission intensity slightly increased in all IMs as compared to their respective parent NIR compound in ethanol except for [DOX][IR783] as shown in FIG. 22D. However, significant decrease were observed in fluorescence emission intenstity of NIR dyes for [DOX][ICG] and [DOX][IR783] in the nanoparticle form in comparison to their parent NIR dyes at 710 nm excitation wavelength, as shown in FIG. 3B. This suggest substantial changes in the photophysical properties of chemo-PTT combination drug when converted from IMs to nanomedicines. The significant decreased in fluorescence emission intensity of two INMs is suggesting an increase in non-radiative vibrational relaxation process. However, a slight increase in fluorescence intensity was observed for [DOX][IR820]INMs. To better understand these changes in the photophysical properties, the FRET parameters and photophysical rates (radiative and non-radiative rate) were calculated.

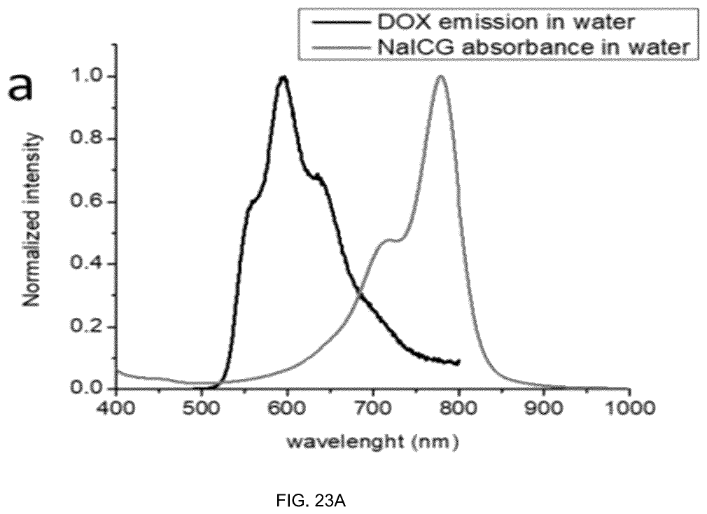

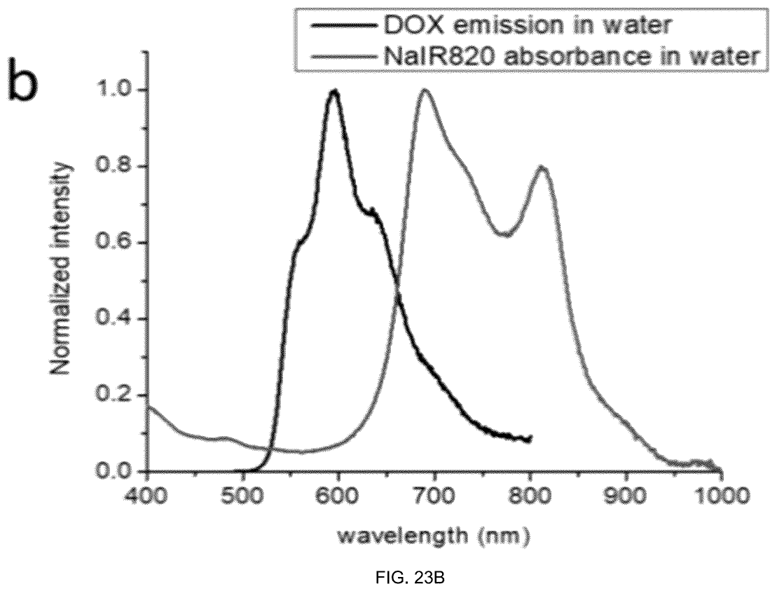

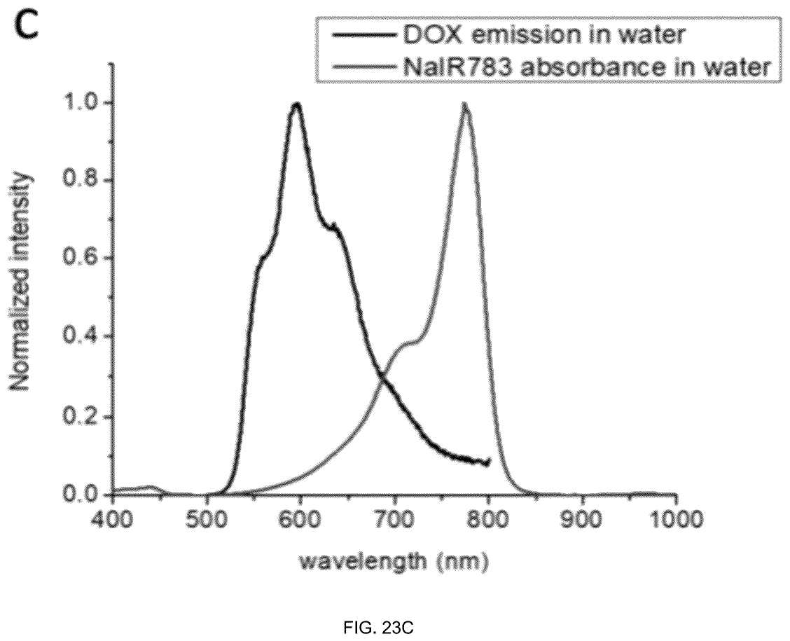

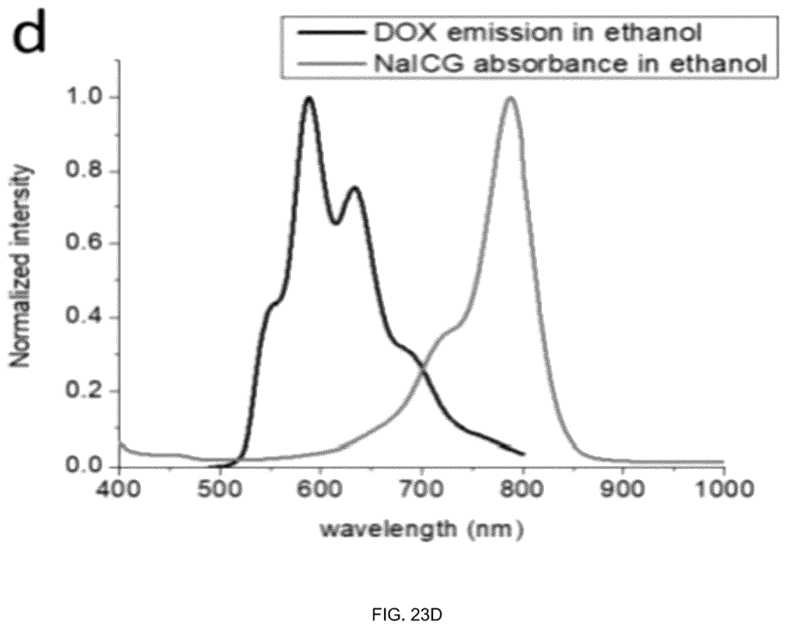

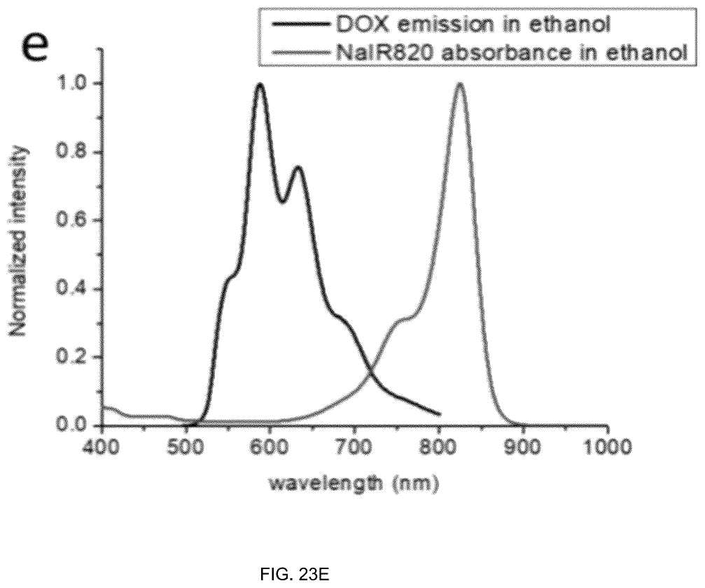

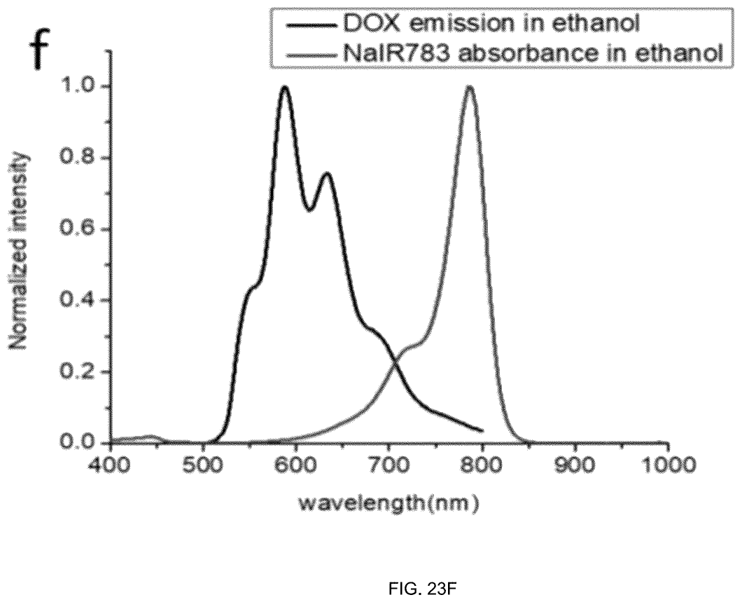

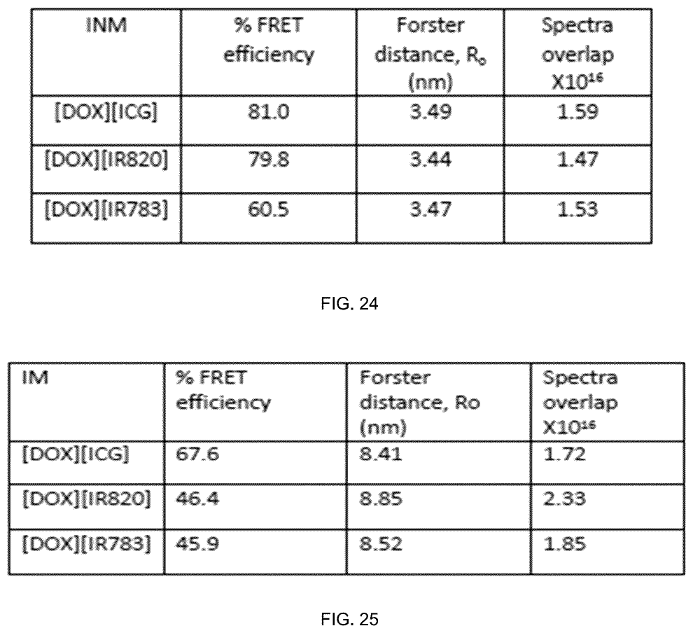

To verify the FRET possibility in the newly developed chemo-PTT IMs and INMs, FRET calculations were performed. FRET is a non-radiative energy transfer from a donor molecule to an acceptor molecule that is within 10 nm distance of each other. In this case, DOX represents the donor while the PTAs are the acceptor molecule. Spectral overlap of donor fluorescence emission and acceptor absorption spectra are depicted in FIGS. 23A-23F for all INMs and IMs in water and ethanol respectively. FRET efficiency and other FRET parameters were calculated using Equations S1-S3 in SI below, and results were shown in FIGS. 24-25.

$$E = 1 - \text{?} \qquad ( S1 )$$

$$\text{?} \text{indicates text missing or illegible when filed}$$

where Fda is integrated fluorescence emission of the donor (DOX) in the presence of the acceptor (TPP dye) and Fd is the integrated fluorescence emission of the donor (DOX) in the absence of the acceptor.

$$J ( λ ) = \text{?} \qquad ( S2 )$$

$$\text{?} \text{indicates text missing or illegible when filed}$$

Equation S2 quantifies the spectral overlap integral (J(λ)) where ϵ(λ) is the molar extinction coefficient (M⁻¹ cm⁻¹) of acceptor (NIR dye) at overlap wavelength λ, and f(λ) is the normalized fluorescence intensity of DOX at overlap wavelength with NIR dye when excited at 480 nm.

$${R}_{0} = 0.0211 ( {n}^{- 4} \times {k}^{2} \times {Φ}_{d} \times J ) \text{?} \qquad ( S3 )$$

$$\text{?} \text{indicates text missing or illegible when filed}$$

R₀ represents the Forster distance in IM or INM, where |Φ_{d} is quantum yield of the donor (DOX) in the absence of the acceptor n is refractive index of the media; J is the spectral overlap integral between donor and acceptor and k² is the dipole orientation factor.

Our group was the first one who reported the existence of FRET mechanism in IMs. The FRET results revealed that the newly developed INMs and IMs exhibited FRET efficiency which was greater than 45%. Moreover, FRET efficiency is significantly higher in INMs as compared to IMs.

Fluorescence quantum yield (FLQY) and photophysical rate constants: The detailed study of absorption and fluorescence emission suggested tremendous changes in the electronic states of the compounds. Fluorescence quantum yield (FLQY) for parent drugs, IMs and INMs were all quantified using a relative technique (Equation S4 below). FLQY was calculated at both 480 nm and 710 nm excitation wavelength for DOX and the NIR dyes respectively. Furthermore, radiative and non-radiative rates were calculated using Equations S5 and S6 below. Fluorescence quantum yield of the donor (DOX) was determined using the relative method as shown in Equation S4. The quantum yield is determined with relative to a standard sodium fluorescein (NaF), with a literature reported quantum yield value of 0.92 in water.

$$Φ \text{?} = {Φ}_{std} * \text{?} * \text{?} * \text{?} \qquad ( S4 )$$

$$\text{?} \text{indicates text missing or illegible when filed}$$

where Φ_{std} is the quantum yield of the standard, I is the integrated emission intensity. Abs is the absorbance at the excitation wavelength (480 nm), and n is the refractive index of the standard (std) and unknown.

where Φ_{std} is the quantum yield of the standard, is the integrated emission intensity. Abs is the absorbance at the excitation wavelength, and n is the refractive index of the standard (std) and unknown(un). ICG was used as a standard with a reported FLQY value of 0.142² in ethanol

From the absorption and fluorescence emission spectra, the radiative rate constant (k_{red}) was also

$${k}_{rad} = 2.88 * {10}^{- 9} * \text{?} * \text{?} \qquad ( S5 )$$

$$\text{?} \text{indicates text missing or illegible when filed}$$

where I represent the emission intensity, v̅ is the wavenumber of light and ε is the molar extinction coefficient.

Using the radiative rate constant, the non-radiative rate constant (knon-rad) was calculate for each compound using Equation S6.

$${Φ}_{F} = \text{?} \qquad ( S6 )$$

$$\text{?} \text{indicates text missing or illegible when filed}$$

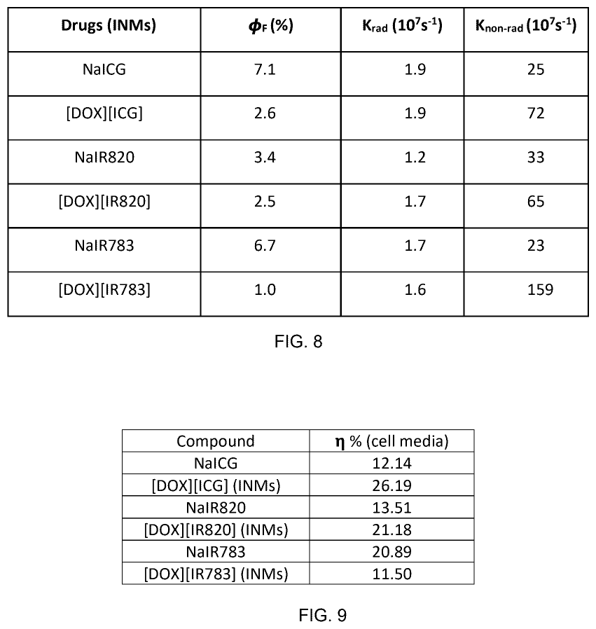

FLQY values for all INMs and IMs in water and ethanol, recorded at 710 nm excitation wavelength, are listed in FIG. 8 and FIGS. 18A-18C respectively.

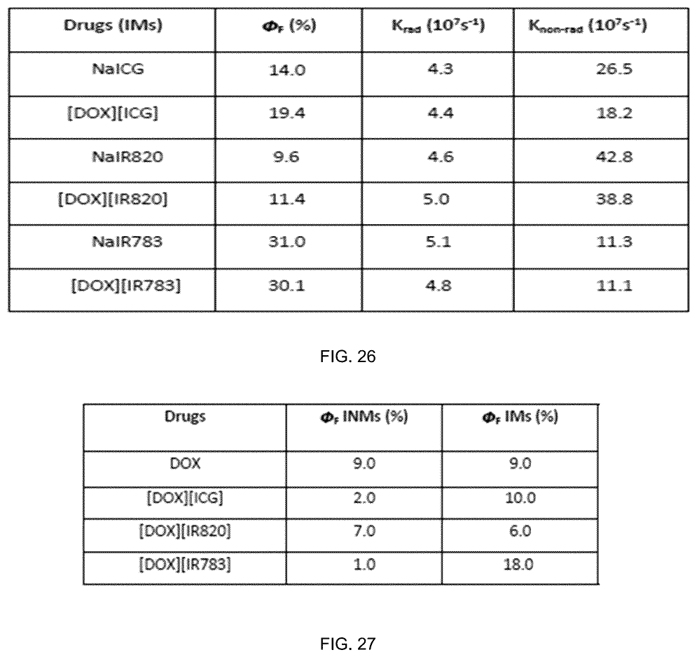

FLQY of IMs increased in comparison to the parent NIR dye in ethanol at 710 nm excitation wavelength except for [DOX][IR783]IMs as shown in FIG. 26. However, the combination drugs in nanoparticle form showed a decrease in FLQY as compared to the parent NIR dyes in water as shown in FIG. 8. Decrease in the FLQY of INMs was likely due to the existence of other dominating non radiative relaxation pathway (knon-rad). This is evident from the quantitative results that knon-rad obtained for the INMs is greater than their radiative rate (krad). The higher knon-rad of INMs depicted that the excited state compound prefers to relax to the ground state via internal conversion or intersystem crossing route. Detailed examination of these results demonstrated that INMs are promising nanomedicine for phototherapies, which exhibited low FLQY owing to an increased non-radiative process necessary for heat generation as well as for ROS generation. Intriguingly, knon-rad for all INMs was significantly improved as compared to their respective parent NIR dyes. To further investigate the potential of INMs, the fluorescence lifetime and photostability experiments are designed.

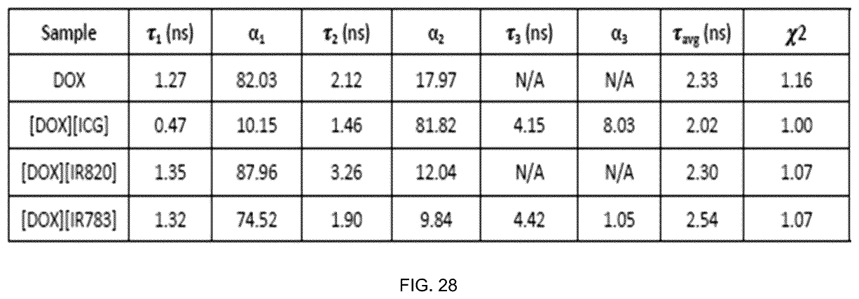

Fluorescence lifetime: The fluorescence lifetime of the chemo-PTT IMs was also investigated in ethanol and the data was fitted bi- or tri-exponentially with χ² values close to unity as shown in FIG. 28 in SI. Fluorescence lifetime fitting correlates well with the previously reported mean fluorescence lifetime of DOX in ethanol. Interestingly, DOX has been found to exhibit an excitation-dependent fluorescence behaviors in different solvent which could consequently affect DOX fluorescence lifetime. However, fluorescence lifetime results for IMs in ethanol excited at 455 nm revealed mainly two states τ₁ and τ₂ (shorter and longer lifetime components) contributing to the fluorescence emission for parent DOX and [DOX][IR820]IM only. For both [DOX][IR783] and [DOX][ICG]IMs, a third lifetime component with the longer lifetime (τ₃) with low abundance (α₃) was observed. Overall quantification of the mean lifetimes for all chemo-PTT IMs in ethanol revealed a shorter-lived excited singlet state for the modified [DOX][IR820] and [DOX][ICG]IMs compared to parent free DOX. Only [DOX][IR783]IM exhibited a longer fluorescence lifetime.

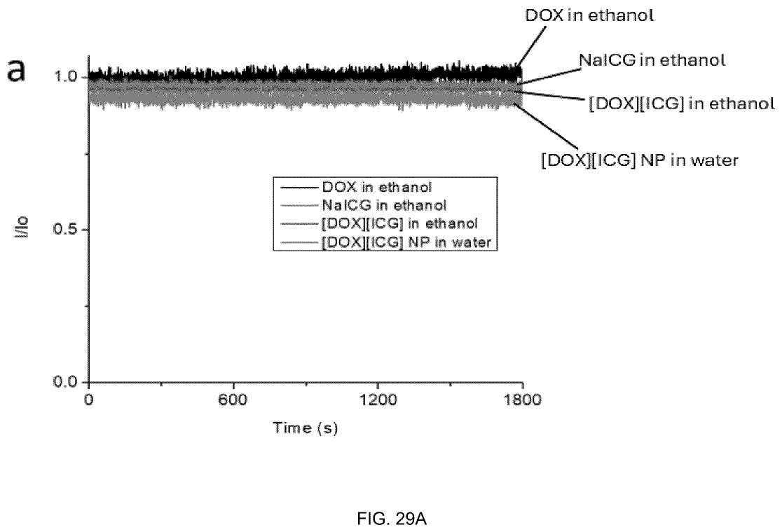

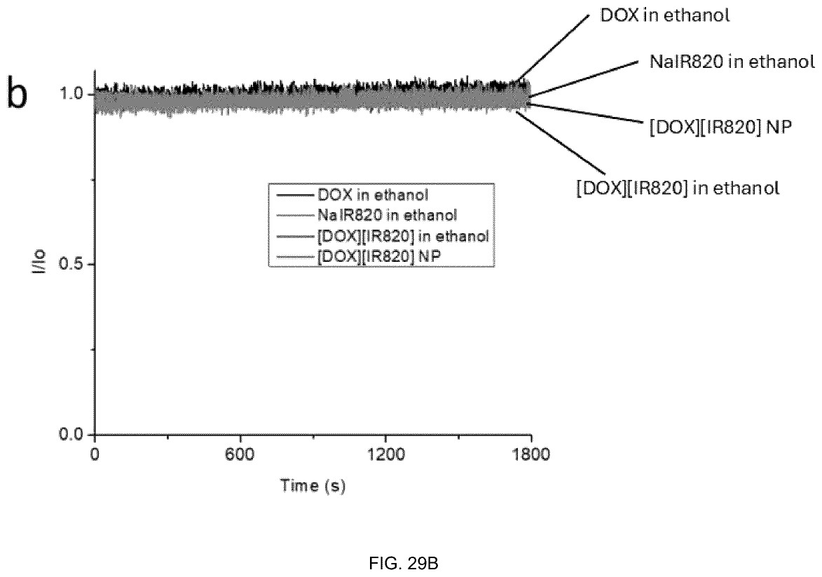

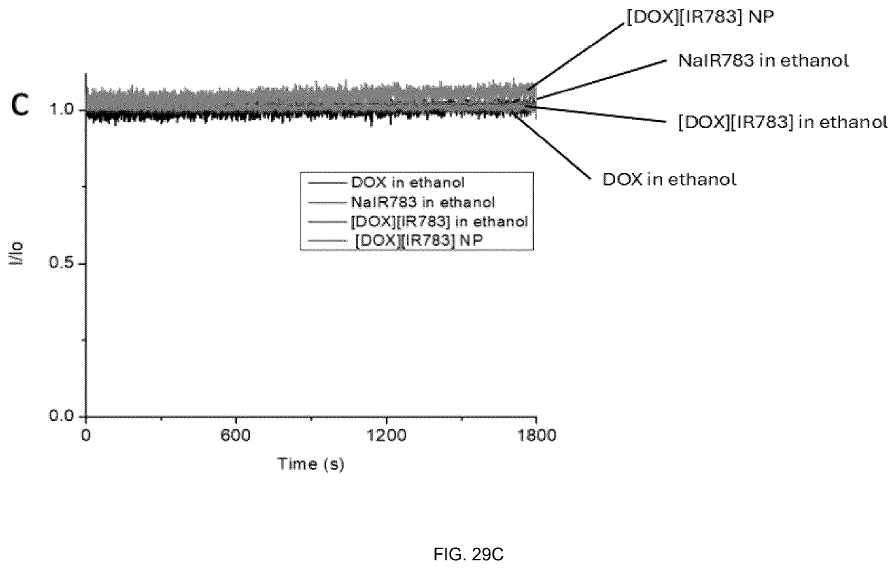

Photostability: Photostability is one of the most important characteristics of a photosensitizer. The photostability of compounds were investigated by recording the fluorescence emission of all IMs and INMs over 30 mins while continuously irradiating at their respective excitation wavelength using the excitation and emission slit width of 14 nm. The examination of results revealed that all forms of the combination drug (IMs and INMs) were photostable for the irradiated time, as shown in FIGS. 29A-29C.

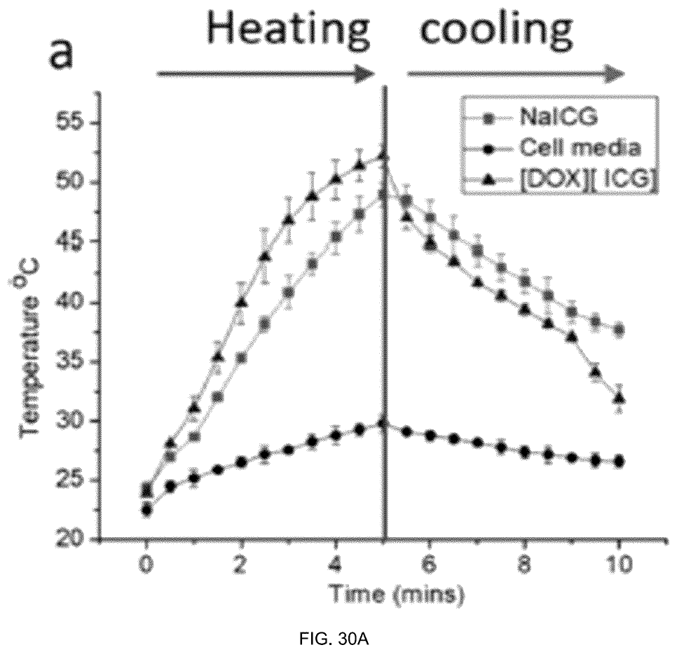

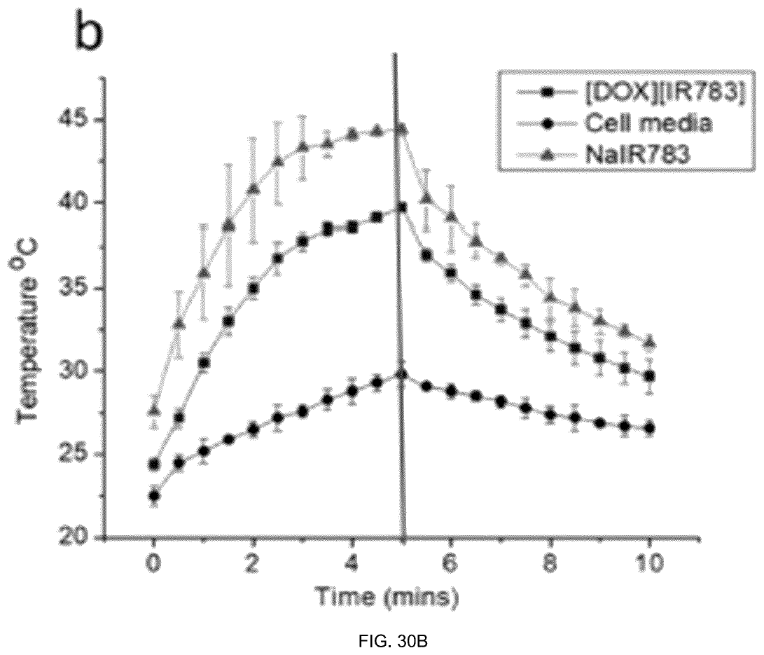

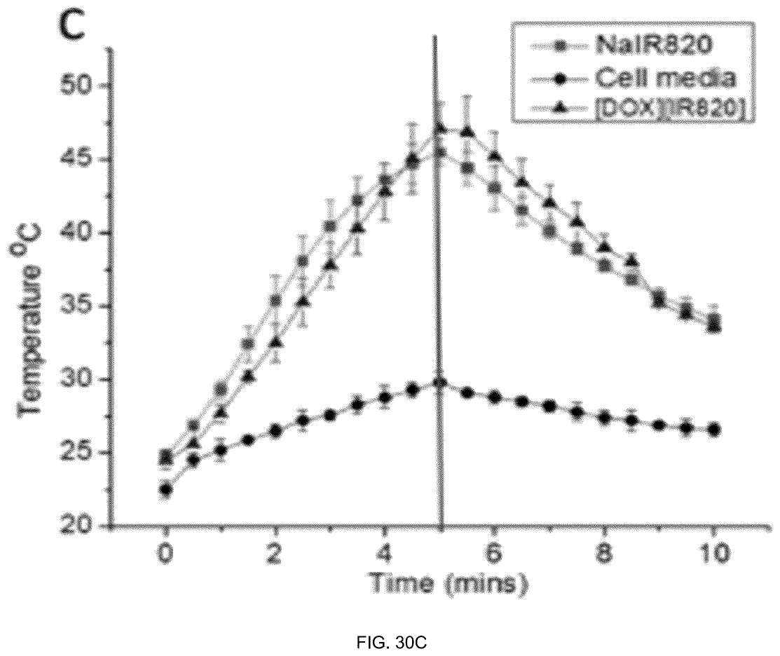

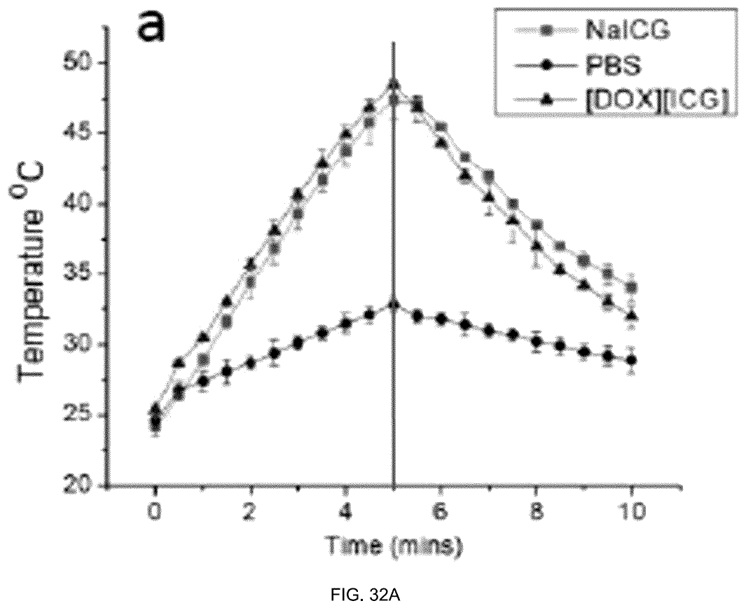

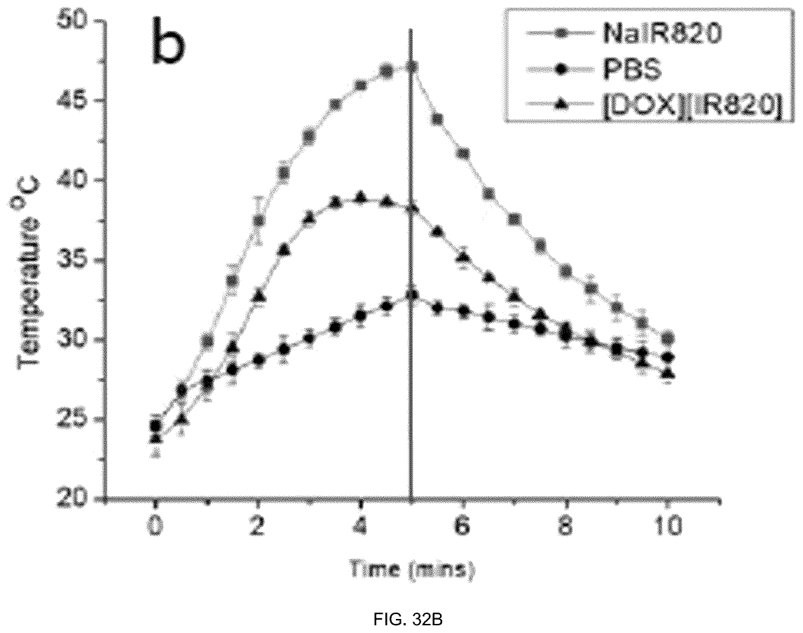

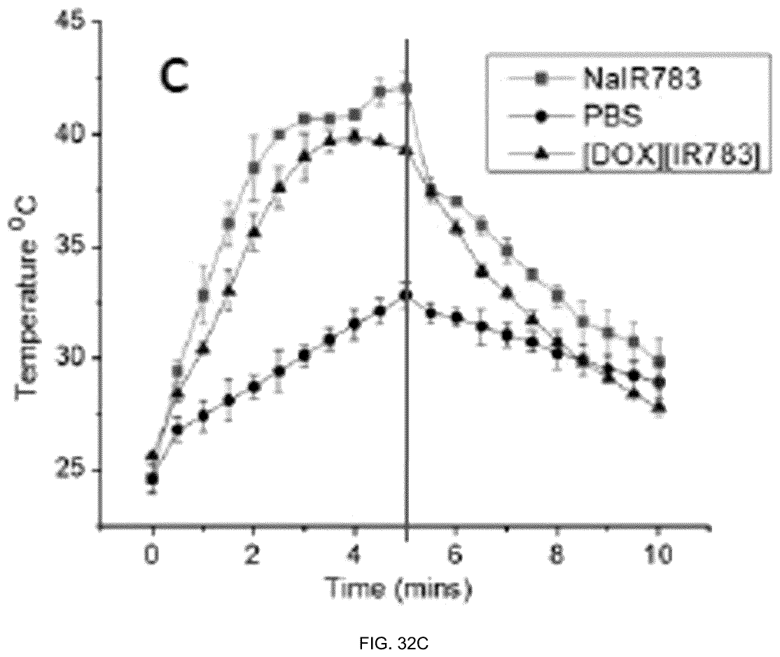

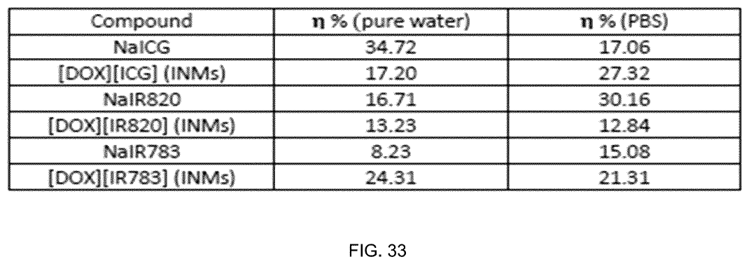

Light to heat conversion efficiency: The light to heat conversion efficiency of all PTAs were investigated to determine their photothermal therapeutic potential. The increase in temperature upon irradiation of PTAs solution with light is monitored to compute the light to heat conversion efficiency of each compound. The results obtained for the NIR parent dyes and INMs were juxtaposed to analyze the changes in the PTT performance of the parent NIR dyes when converted into a combination ionic drug. This experiment was performed in different solvents (cell culture media, pure water and PBS) using 808 nm laser as an excitation source, to explore the PTT behavior of the drugs in different aqueous environment. The heat efficiency graphs are shown in FIGS. 30A-32C. Most PTAs including combination INMs exhibited an increase in temperature, indicating that sufficient heat was generated when irradiated with 808 nm laser light source. The light to heat conversion efficiency was quantified using Equations S7-S10 below and the results summarized in FIG. 9 and FIG. 33.

Photothermal heat conversion efficiencies (η) of the INMs and parent drugs were determined using the following S7-S10.

$$η = \text{?} \qquad ( S7 )$$

$$\text{?} \text{indicates text missing or illegible when filed}$$

Where h is the heat transfer coefficient, s represents the surface area of the container, and hs is obtained from Equation S10 and FIG. S10. Tₘₐₓ is the steady state temperature of the INMs and for [DOX][IR783]it was found to be 44.5° C. (FIG. S10c). The environmental temperature (Tₛᵤᵣᵣ) was 22.5° C. The change in temperature (Tₘₐₓ-Tₛᵤᵣᵣ) for [DOX][IR783] INM was determined to be 22.0° C. I indicate the laser power, which was 1 W for all samples. A represents absorbance of PTS. Qₐᵢₛ is the heat dissipated from lights absorbed by solution and cuvette walls Qₒₗₛ for INMs sample was determined from sample control with pure cell media and was found to be 17.2 mW. To determine hs, the following dimensionless parameter, θ, is introduced in Equation S8.

$$θ = \frac{T - {T}_{max}}{{T}_{max} - \text{?}} \qquad ( S8 )$$

$$\text{?} \text{indicates text missing or illegible when filed}$$

Then, the time constants τ3 can be deduced from Equation S9.

$$t = \text{?} \qquad ( S9 )$$

$$\text{?} \text{indicates text missing or illegible when filed}$$

Then, the time constant Σs of the [DOX][IR783] INMs sample was determined to be 287.5s. By inserting this value into Equation S10, hs can be calculated.

$$hs = \text{?} \qquad ( S10 )$$

$$\text{?} \text{indicates text missing or illegible when filed}$$

where m_{D} is the mass of solution (1.0 g) and C is the specific heat (4.2 J/g° C.). hs for [DOX][IR783] was determined to be 14.6 mW/° C. After substituting all parameters into Equation S4, η was determined to be 24.3%.

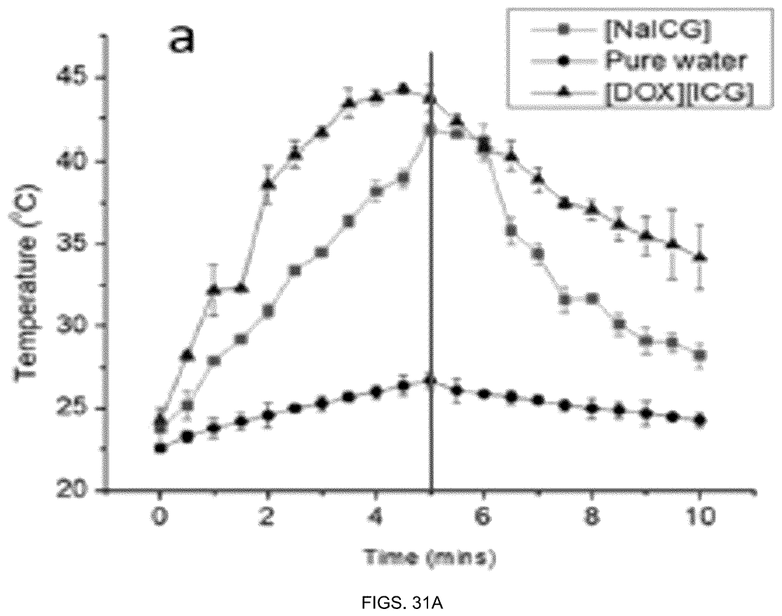

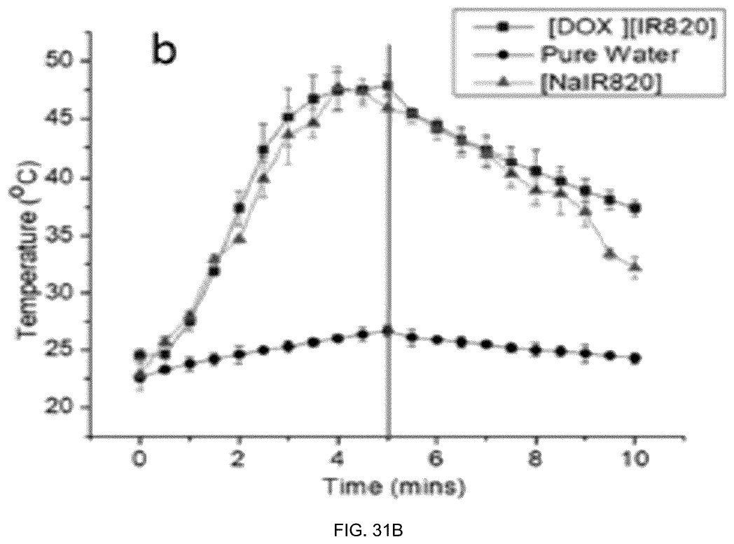

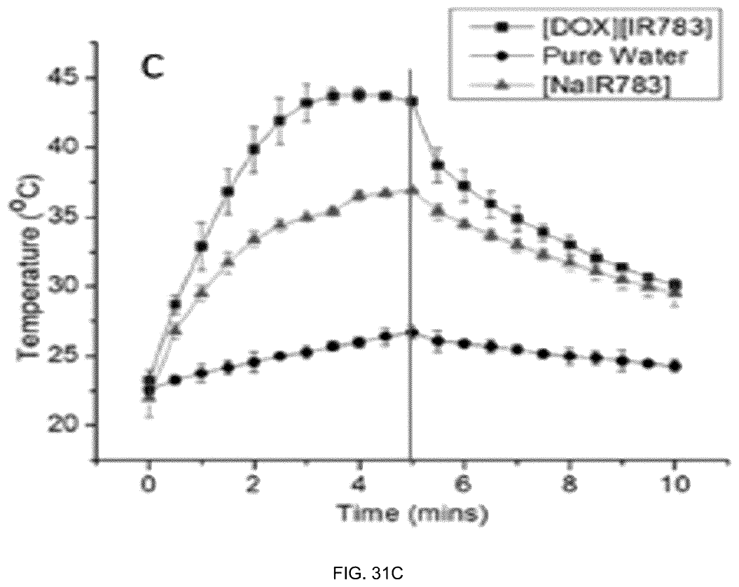

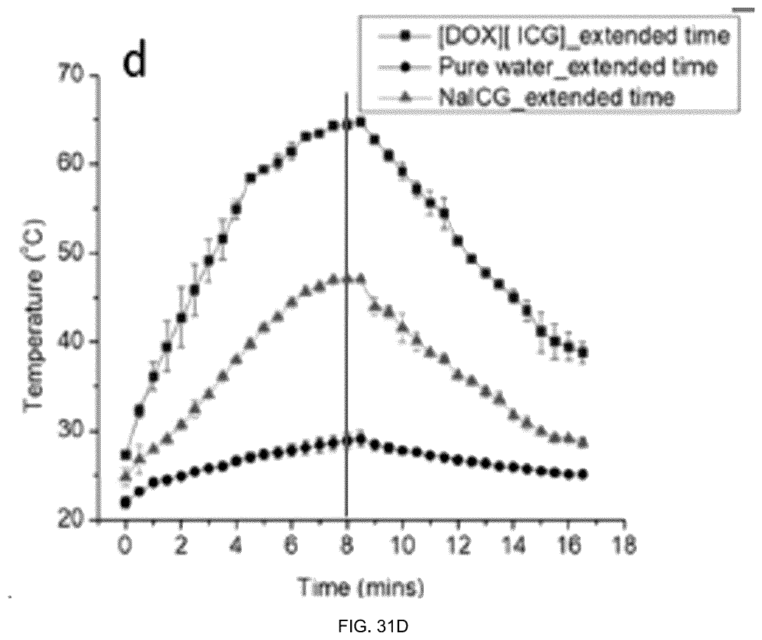

The detailed examination of the heat efficiency data revealed that all INMs photothermal therapeutic potential is dependent on the media. In cell culture media, all INMs showed enhanced PTT effect as compared to their respective NIR dyes with exception of [DOX][IR783]. Interestingly, [DOX][IR783]INM exhibited a greater heat efficiency as compared to the free NIR dye in both water and PBS, which contradicts the cell culture media results. In contrast, [DOX][ICG] and [DOX][IR820]INMs showed lower value for light to heat conversion efficiency than their respective parent NIR dyes in water media (FIGS. 31A-31D). This could be attributed to a better cooling curve generated for parent NIR dyes under similar experimental conditions. A decrease in the photothermal conversion efficiency for INMs as opposed to parent NIR dye under similar experimental condition does not necessarily mean that the INM lacks PTT potential. It still possesses PTT characteristics, but it did not improve in the INMs form. However, to increase the PTT efficiency of any drug requires optimization such as adjustment of the laser power and exposure timing of the PTA to irradiation. Therefore, light to heat conversion efficiency experiment was conducted over a longer period (8 mins) to investigate the PTT changes in [DOX][ICG]INM and ICG behavior at longer heating and cooling time range in water (FIG. 31D). This led to an increased light to heat efficiency (33.2%) for [DOX][ICG]INM as opposed to the parent NaICG dye (27.5%). Although, light absorbance behavior of NaICG did not significantly differ from the results acquired for 5 mins irradiation experiment. Whereas a drastic change in temperature (Tₘₐₓ-Tₛᵤᵣ) from 37.5° C. to 64.75° C. was observed for [DOX][ICG]INM during the 8 mins experiment. This signifies the flexibility in PTT capability of INMs upon modification of the parameters to attain desired photothermal therapeutic effect. Even though a higher temperature change was observed for [DOX][ICG]INM at extended time, it should be noted that a temperature greater than 49° C. have been shown to result in necrotic cell death.

Intriguingly, an improved PTT performance was exhibited by [DOX][ICG]INMs in PBS and cell culture media as compared to the free PTT NaICG dyes. Literature reported value for the specific heat capacity of PBS (1×) and cell culture media are 3.85 kJ/kgk and 4.18 kJ/kgK respectively which suggest the effect of media.

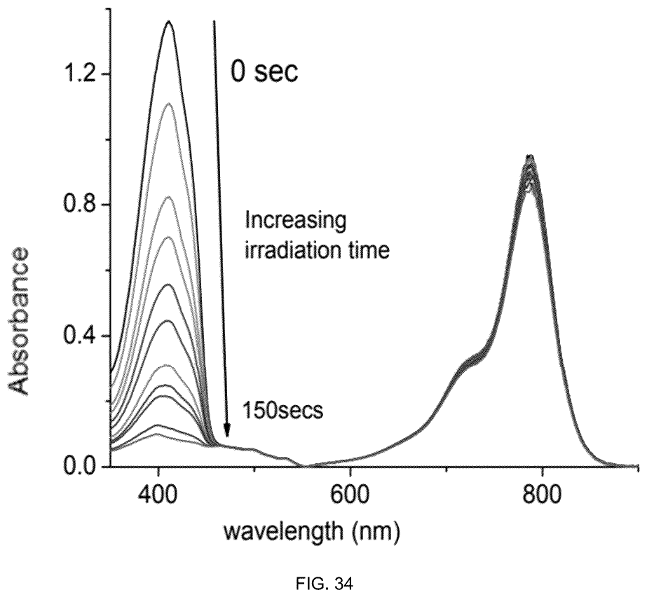

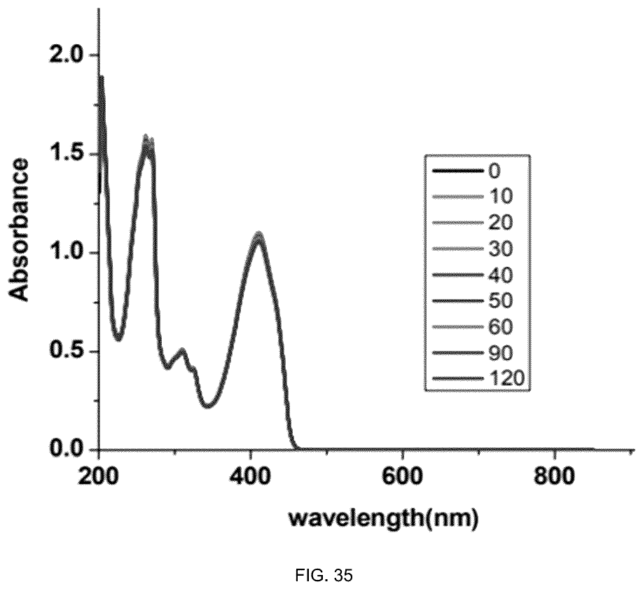

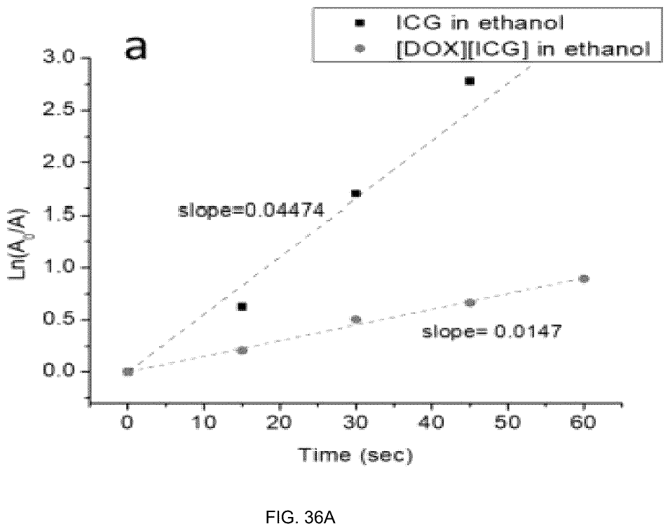

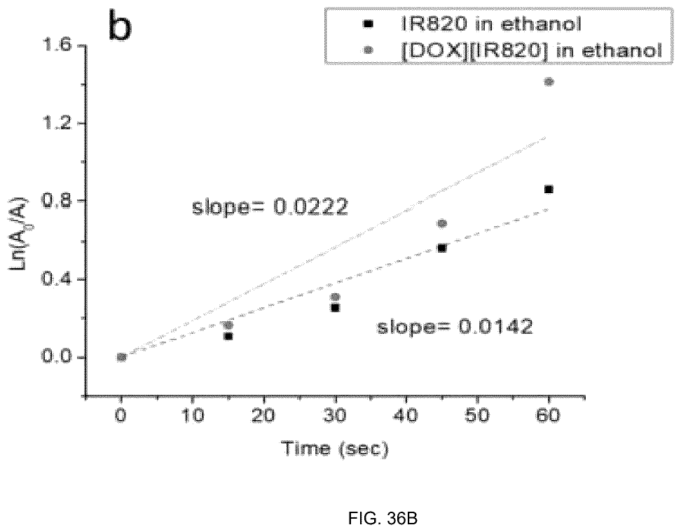

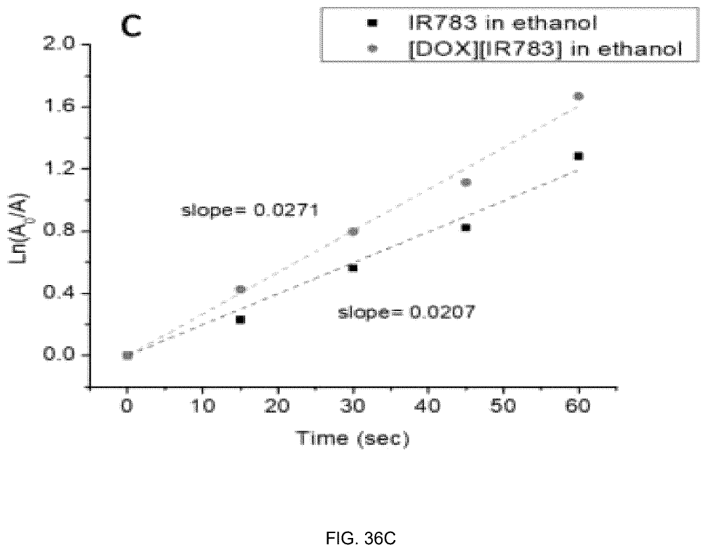

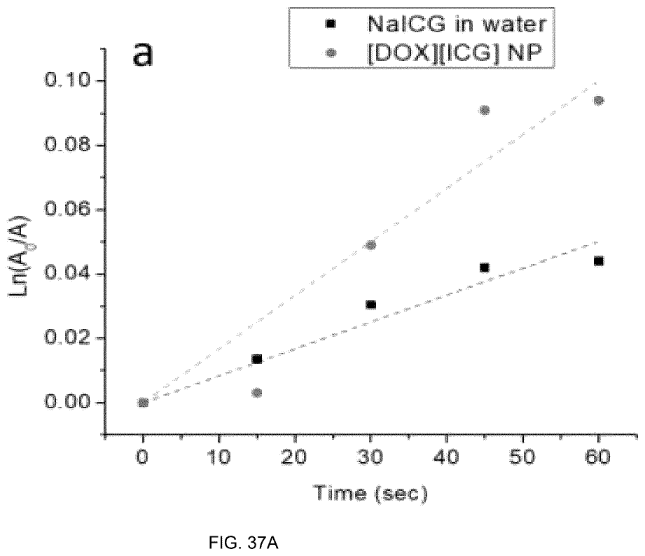

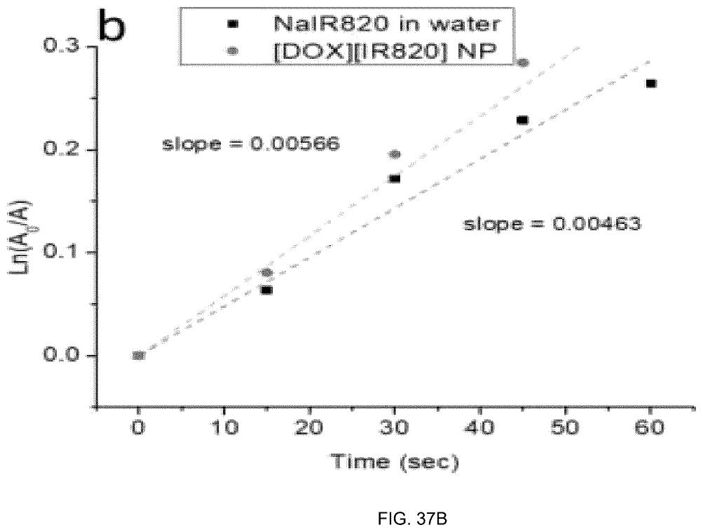

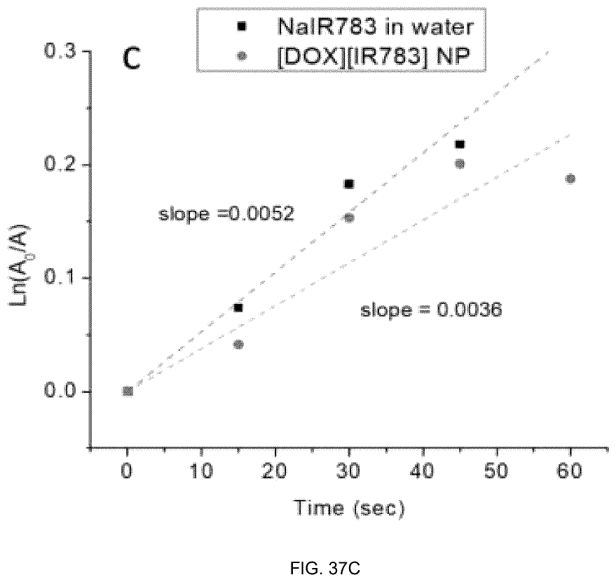

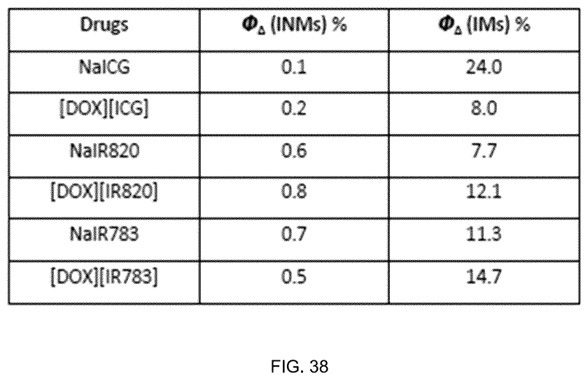

Singlet oxygen quantum yield (SOQY): There are few reports that mentioned the PDT effect of NIR dyes. Therefore, ROS quantum yield experiment is designed to quantify the alterations in ROS production of NIR dye upon changes in the counter-cation. SOQY was quantified by recording the rate of decrease in absorbance of DPBF upon irradiation using 808 nm laser. FIG. 34 shows the decrease in DPBF absorbance with increasing irradiation time in the presence of [DOX][ICG] in ethanol. A similar control experiment was designed to investigate the changes in absorbance of DPBF only after irradiation with 808 nm laser, and the spectra at different irradiation times was shown in FIG. 35. This experiment proved that DPBF absorbance only decreased under 808 nm laser irradiation in the presence of INMs. The change in absorbance of the probe was analyzed and used to compute the SOQY for all IMs and INMs which are shown in FIGS. 36A-37C respectively. In water, [DOX][IR820] and [DOX][ICG]INMs exhibited a significant increase in SOQY when compared with the free drug as shown in FIG. 38. This result is very well correlated with the increased K_{non-rad}. Since intersystem crossing (ISC) is also a form of K_{non-rad}, the increased K_{non-rad} for both [DOX][IR820] and [DOX][ICG]INMs signifies a contributive effect of ISC and internal conversion (FIG. 8). However, [DOX][IR783]showed a decrease in SOQY which may be ascribed to the effect of enhanced light to heat conversion efficiency exhibited by the [DOX][IR783]INMs. In addition, due to the competing pathways between two non-radiative relaxation processes, it is expected that a decrease in SOQY caused an increase in heat generation. Although the SOQY of the INMs fall within range of 0.2-0.8% in water, [DOX][ICG] and [DOX][IR820]INMs showed improved SOQY as compared to the free parent dyes. However, [DOX][IR783]INM did not follow the same trend. It could be attributed to the nano formulation of the drug.

For IMs in ethanol, only [DOX][IR783] and [DOX][IR820]was found to exhibit a higher SOQY in comparison with the parent free drug (FIGS. 23A-23F), possibly due to higher K_{non-rad} (ISC rate) as opposed to k_{rad}. However, [DOX][ICG]SOQY in ethanol was significantly decreased which could probably be ascribed to its increased radiative rate, decreased non radiative rate and high fluorescence quantum yield (Table S6). Generally, the presence of DOX cation introduced to the PTT dye significantly altered its SOQY efficiency.

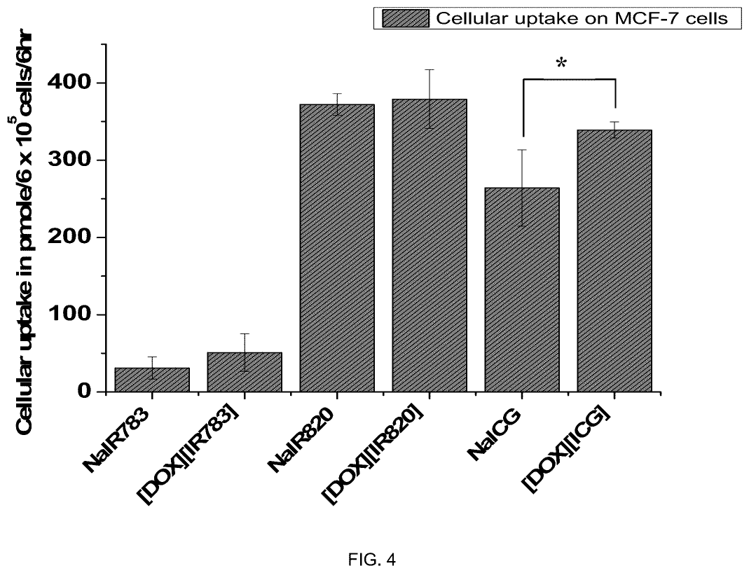

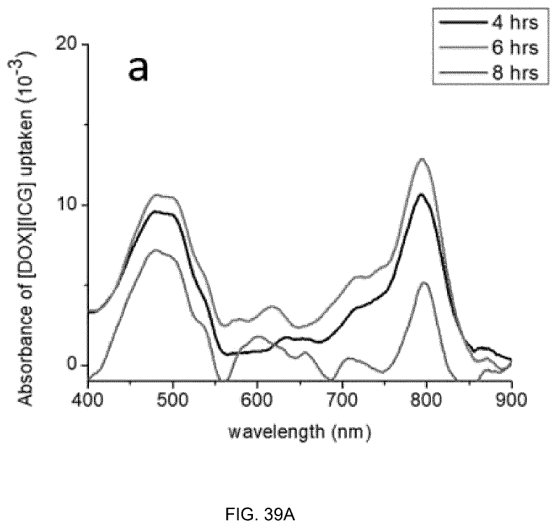

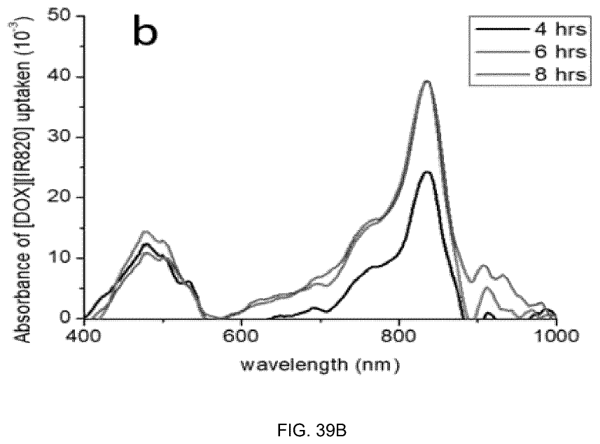

Cellular uptake: To prove these INMs potential as a combination nanomedicine for cancer, several in vitro experiments were designed. Since the nanomedicine morphology is different than the parent soluble drugs which can impact the cellular uptake of the drug, therefore cellular uptake experiments were performed using MCF-7 breast cancer cells. The results from cellular uptake study of the INMs as compared to the NIR dyes after 6 hr of drug incubation are shown in FIG. 4. The enhanced concentration of the INMs over parent drugs in cells, was quantitatively measured via a UV Visible spectrophotometer using a reported protocol. Time dependent cellular uptake experiment showed maximum uptake for both [DOX][ICG] and [DOX][IR820] in MCF-7 cells at 6 hr, as depicted in FIGS. 39A-39B. The cellular uptake of [DOX][ICG]INMs is enhanced relative to NaICG parent dyes (FIG. 4). In contrast, [DOX][IR783] and [DOX][IR820] only showed a slight enhancement in the cellular uptake, probably due to the morphology of nanoparticles. The low cellular uptake of [DOX][IR783] is attributed to its higher PDI.

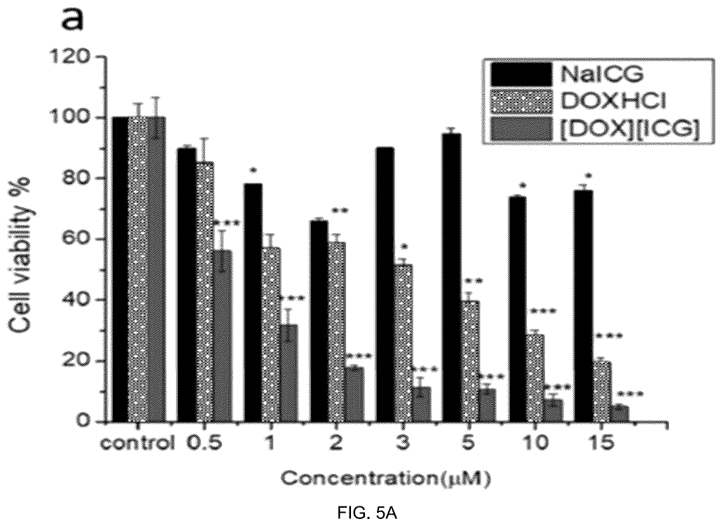

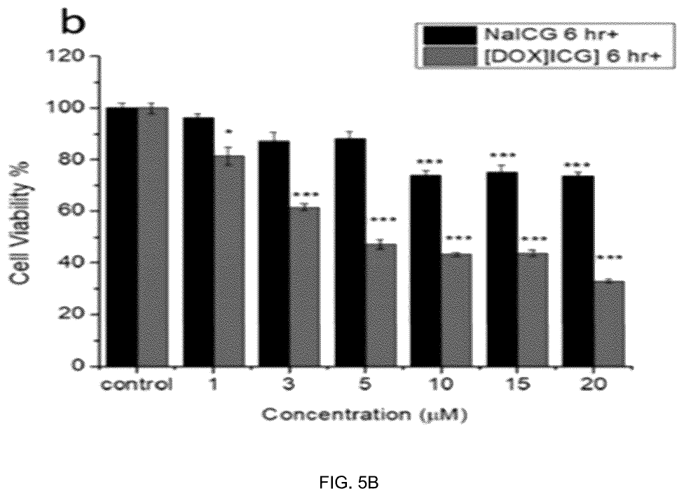

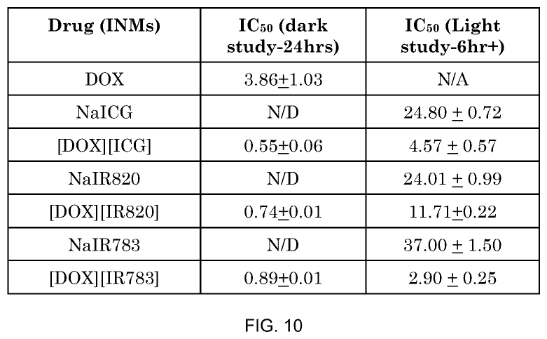

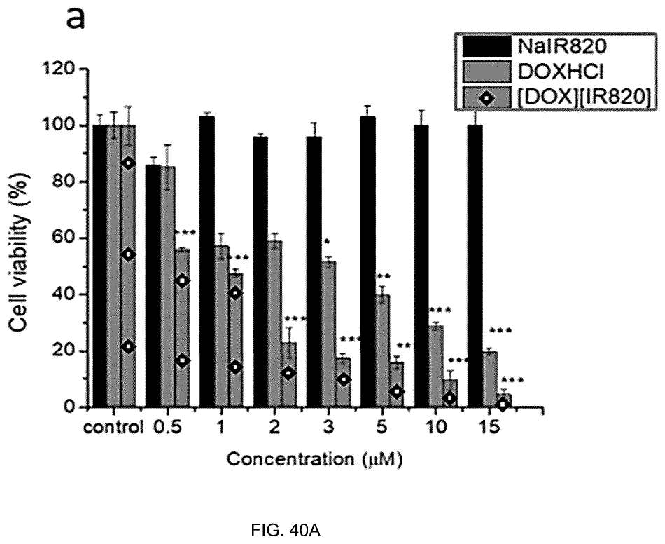

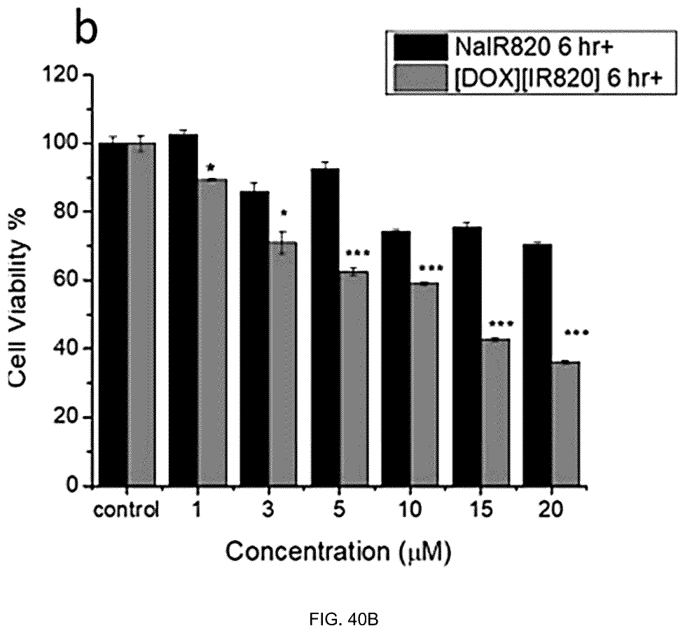

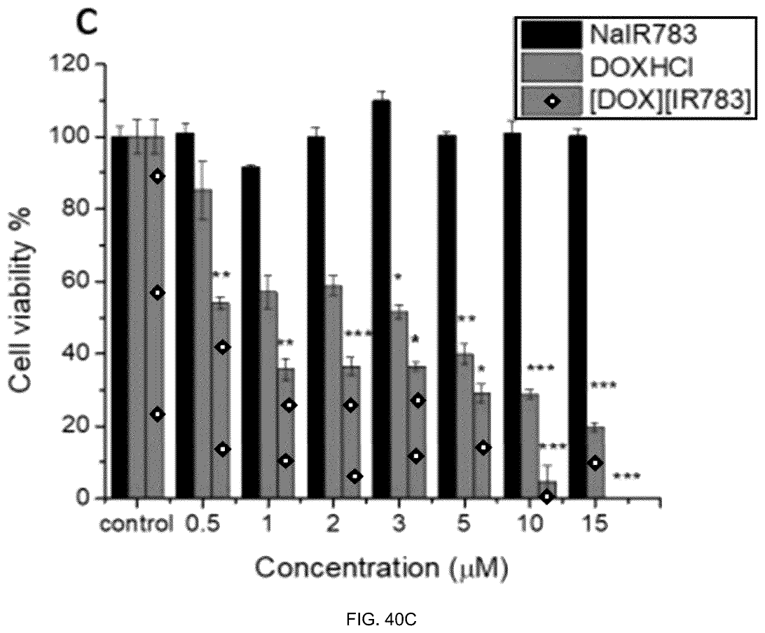

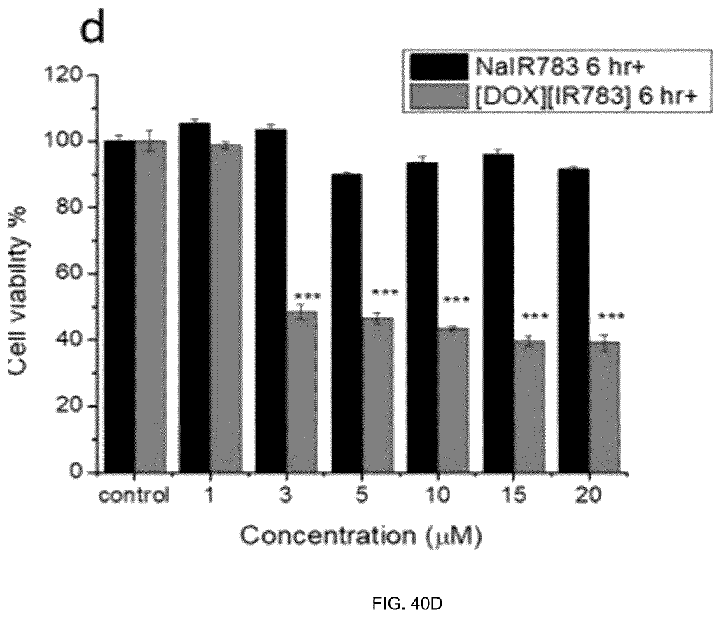

In vitro cellular Toxicity of INMs: In vitro cytotoxicity of the INMs was performed to assess the drug potency as an anti-cancer drug possessing dual therapeutic mechanisms (chemo and photothermal effect) when compared to the free chemotherapy or PTT drugs separately. Half maximal inhibitory concentration (IC₅₀) of the three INMs ([DOX][ICG], [DOX][IR820] and [DOX][IR783]) were compared to their respective parent chemo and NIR dyes (NaICG, NaIR820 and NalR783). In the dark, the parent NIR dyes showed low cytotoxicity as expected. The data for the dark cytotoxicity of the parent compounds and ICG containing INMs are presented in FIG. 5A. Similarly, the dark cytotoxicity results for [DOX][IR820] and [DOX][IR783]INMs showed that the IC₅₀ was greatly lowered as compared to the parent DOX and NIR dyes (FIGS. 40A and 40C). The summarized dark cytotoxicity results for all drugs are also reported in FIG. 10. The toxicity observed in the dark is due to the presence of DOX since the PTT mechanism of the NIR dyes can only be activated in the presence of light irradiation. An improved DOX chemotherapeutic efficacy in the form of combination INMs revealed that DOX toxicity can be changed by tailoring counter anion. In addition, DOX in the form of nanoparticles exhibited lower IC₅₀ in the dark in comparison to the parent FDA approved DOX chemotherapeutic drug due to a better cellular uptake and EPR effect of the tumor. This remarkable finding signifies the potential ability of counterion and nanotechnology to finely tune the potency of DOX. It is possible that the toxicity of the combination drug could be further enhanced by tuning the size of nanoparticles and counterions which is very important to inhibit the side effects of DOX. Since the combination INMs has dual mechanism where the dark toxicity results have proven the improved toxicity of the chemotherapeutic cation in the combination INMs, therefore light toxicity experiments are designed to investigate the photothermal effect of the anion that can only be activated in the light. Thus, the combination nanomedicines were subjected to light irradiation in vitro.

Photothermal effect in vitro: To assess the potential of the INMs for use as a PTAs, a light cytotoxicity experiment of the INMs was designed at 6 hrs using the protocol mentioned in experimental section. Photothermal effect was investigated at the 6^{th} hr due to high cellular uptake of the INMs at that time (FIGS. 39A-39B). The cytotoxicity results demonstrated that the parent NIR dyes were only slightly toxic in the presence of light at lower concentration, but increased cytotoxicity was observed at high concentration (FIG. 41). Intriguingly, the light cytotoxicity results (shown in FIGS. 5B, 40B, and 40D) demonstrated that chemo-PTT combination INMs were more toxic to MCF-7 cells than the parent PTT dyes, possibly due to a higher cellular uptake, higher molar absorptivity, enhanced photothermal conversion efficiency and higher ROS quantum yield. Since both therapeutic mechanisms (chemo and PTT) of the combination INMs will be activated in light, it is therefore crucial to examine the synergy between chemo and PTT mechanisms of the nanomedicines.

Combination Index (CI): Degree of drugs synergism is measured by the CI. In the case of a chemotherapeutic drug complexed with a photothermal therapeutic drug, the significance (additive/antagonistic/synergistic) of the effect of the chemotherapeutic drug in the presence of the photothermal drug is determined by CI. CI is quantified by the Equation 2.

$$CI = \frac{{IC}_{50} ( A + B )}{{IC}_{50} ( A )} + \frac{{IC}_{50} ( A + B )}{{IC}_{50} ( B )} \qquad ( 2 )$$

Where IC₅₀ (A+B) represents the IC₅₀ value for chemo-PTT combination drug and IC₅₀ A or B represents IC₅₀ value of chemotherapeutic drug and PTT drug respectively.

For [DOX][IR783], [DOX][IR820], and [DOX][ICG], the estimated CI values are 0.28, 0.74, and 0.32, respectively. A synergistic effect is observed for all combination nanomedicines since the CI value is lower than 1. A greater synergistic effect was observed for [DOX][IR783]INM with the lowest value of Cl. NaIR783 was observed to be the least effective drug as evident by the IC₅₀ value in the light. However, when combined with DOX, i.e [DOX][IR783], a significant decreased IC₅₀ value was observed which was not recorded for other INMs. This behavior resulted in a greater synergism between the chemo and the PTT drug in [DOX][IR783]INMs.

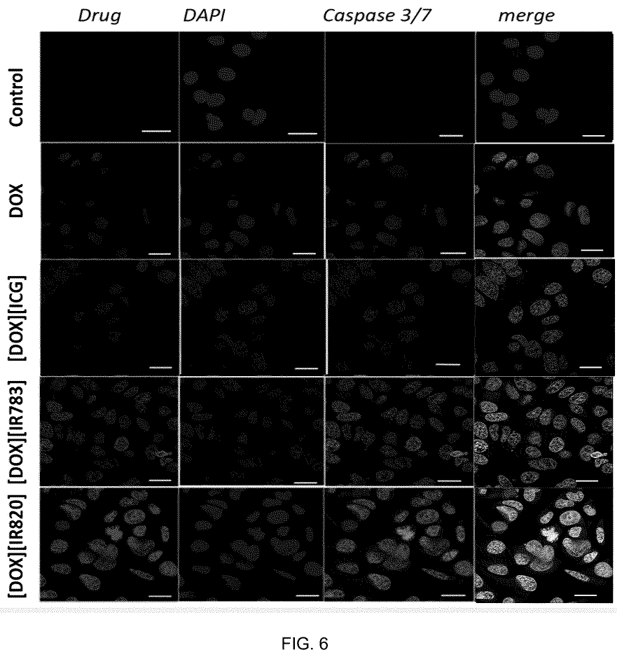

Cell death mechanism: Two experiments, employing caspase 3/7 reagent and flow cytometry were designed to investigate the mechanisms of cell death. Caspase 3/7 reagent serves as a sensor for apoptotic signal. In the presence of live cells, this reagent does not fluoresce. Caspase 3/7 DEVD peptide conjugated to DNA fluorophore cleaves whenever apoptosis signal is detected upon drug treatment. The cleavage leads to the release of the DNA-binding fluorophore to bind to the nucleus of the apoptotic cell, generating a bright green fluorescence emission. As observed from the confocal images in FIG. 6, treatment of the cells with DOX resulted in a bright green fluorescence. The bright green signal observed for DOX is expected as doxorubicin is known to induce of apoptosis. Interestingly, the bright green fluorescence was more prominent for the newly developed INMs, signifying an increased caspase activity. The apoptosis mechanism was further examined using the flow cytometry which offers a more detailed cell death information about apoptosis and necrosis.

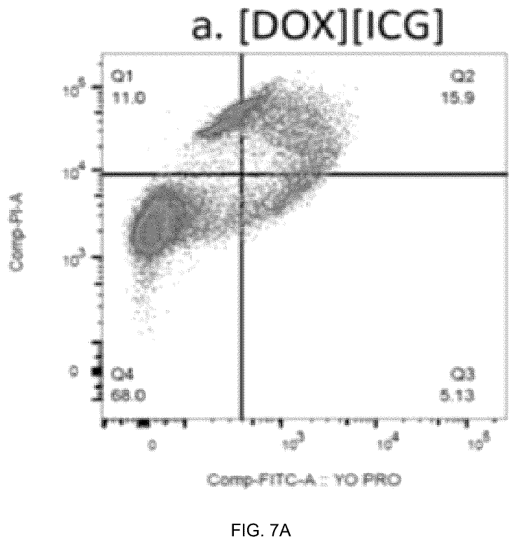

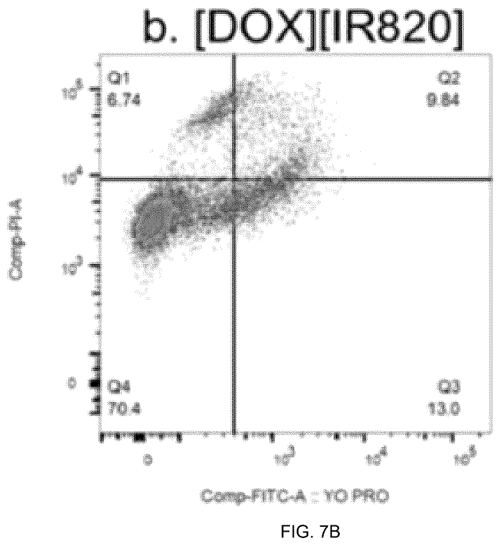

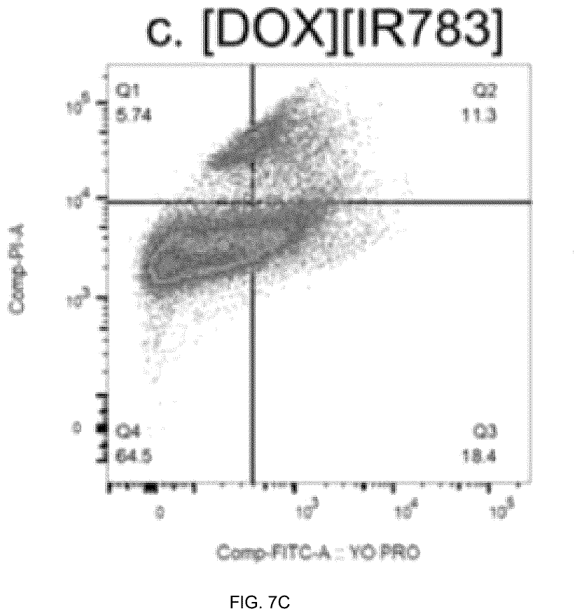

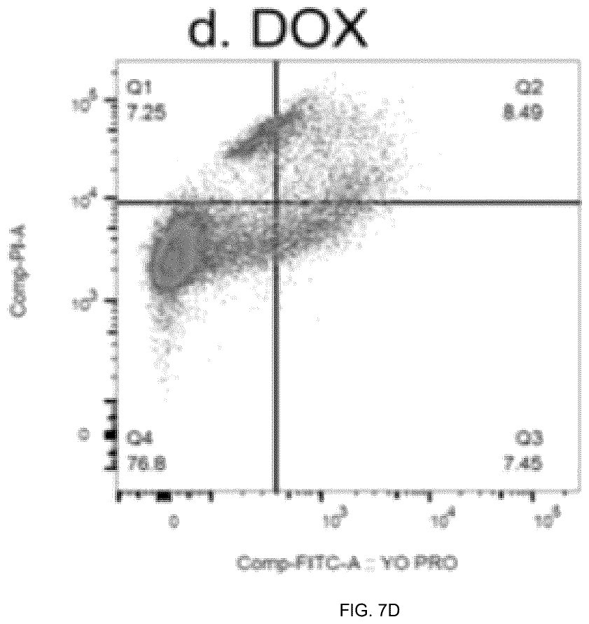

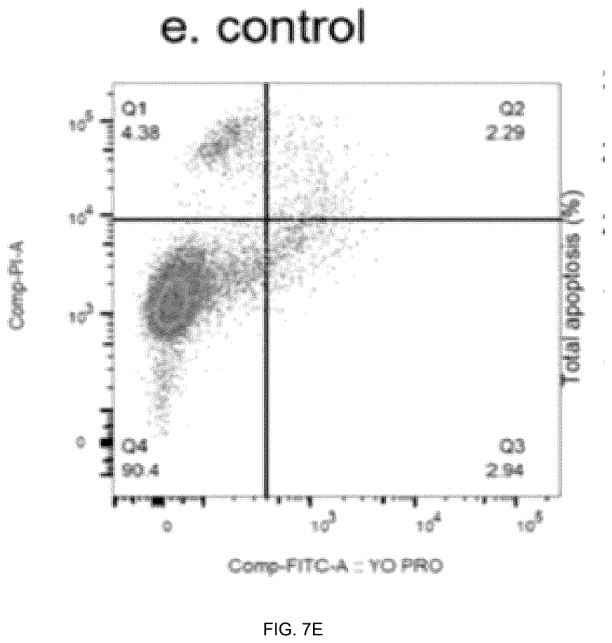

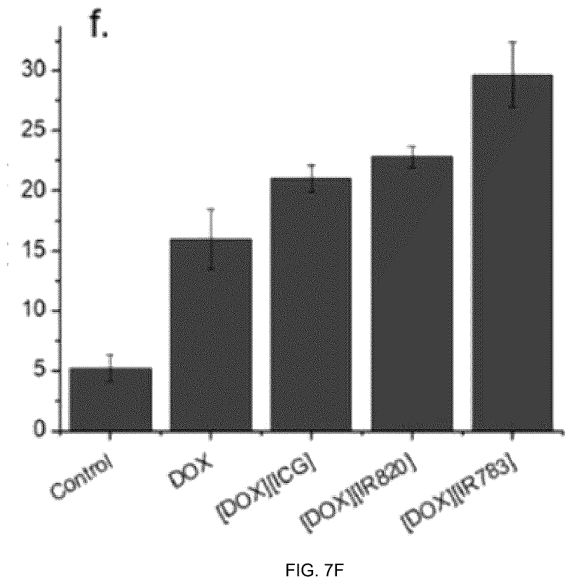

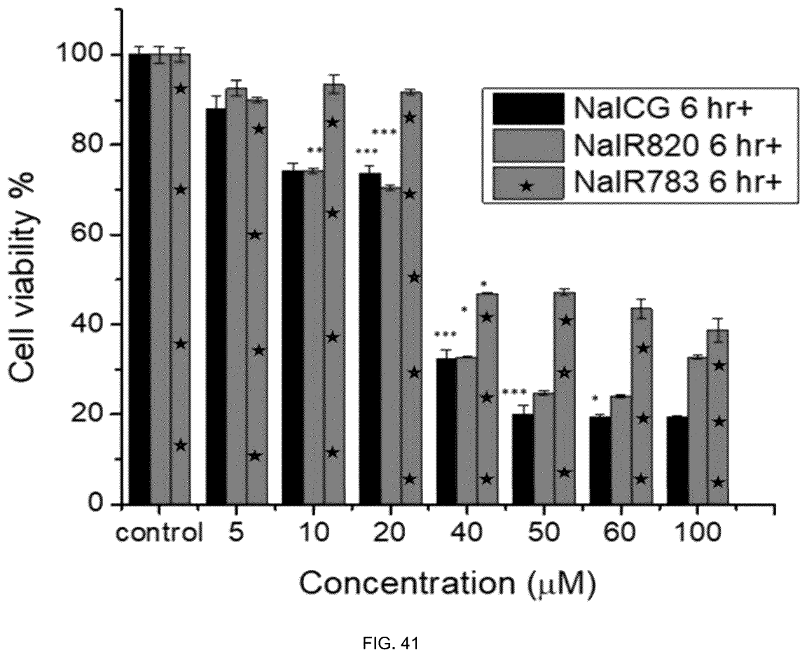

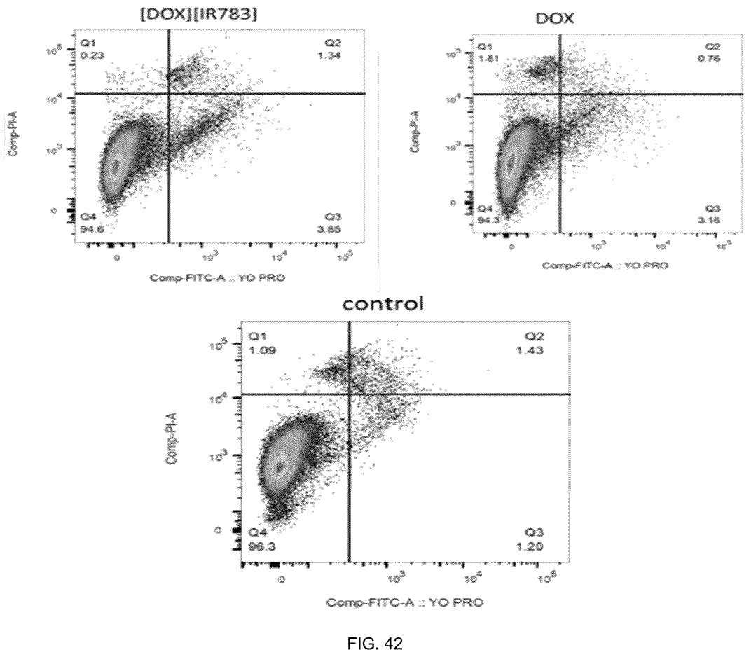

Flow cytometry: Flow cytometry enables a detailed quantitative analysis of the modes of cell death. By staining the cells with YO-PRO and PI (commercially available kit), cells undergoing both apoptosis and necrosis were analyzed. When MCF-7 cells were treated with [DOX][IR783]INM for 6 hr (FIG. 42), percentage of total apoptotic signal (both early and late apoptotic signal added up) slightly increased from 3.9% for DOX to about 5.2%. Necrosis effect was also observed to decrease for the newly developed INM as opposed to DOX treatment. Interestingly, during an increased drug treatment (48 hr) as shown in FIGS. 7A-7D, all INMs treatments resulted in the higher percentage of apoptotic cell death. Importantly, the percentage of total apoptotic signal increased for the three INMs as compared to soluble DOX. It was also observed that apoptosis signal for [DOX][IR783]INM was increased 2-times as opposed to DOX with the percent apoptosis of 15.9% to about 30% for DOX and [DOX][IR783]INM, respectively. Similarly, the necrotic effect observed in all treatment was minimized for most INMs as compared to DOX. This improved cell death apoptotic mechanism could also be attributed to the effect of NIR counter anion and nanoparticle morphology. Thus, it is concluded that DOX apoptotic cell death mechanism can be tailored using IMs chemistry.

### Conclusion

Three distinct combination chemo-PTT IMs were synthesized via simple ion exchange reaction by replacement of the chloride counterions of the chemotherapeutic drug, DOX, with three different PTT active NIR absorbing dyes (NaICG, NaIR783 and NaIR820). By using a simple reprecipitation method, the IMs were modified into carrier free aqueous NPs (INMs). Improved photophysical properties were observed for NIR dyes when sodium counter-cation is replaced with DOX cation in IM and INMs. PTAs' improved molar absorptivity at longer wavelength was only observed in combination IMs and INMs' that make them suitable to treat deep seated tumor due to deeper penetration of light. Intriguingly, all three chemo IMs and INMs also exhibit FRET capabilities. Detailed analysis of photophysical characteristics and comparison of the various radiative and non-radiative rates revealed that the INMs' possessed excellent characteristics to serve as a better PTA and photosensitizer for PDT. The significant increase in light to heat conversion efficiency and ROS generation upon irradiation with 808 nm laser was also observed in combination INMs as compared to parent NIR dyes. In comparison to the routinely utilized single chemo-therapeutic approach, DOX in INMs' exhibited enhanced dark cytotoxicity (more than 4 times) and high cellular uptake towards MCF-7 cell lines. In addition, significantly improved light toxicity of NIR dyes was observed in INMs as compared to parent NIR dyes due to enhanced photothermal conversion efficiency and ROS quantum yield. Moreover, combination index values of less than 1 for all INMs is indicative of the synergistic interaction of both chemo and PTT drugs used in tandem. Analysis from confocal images revealed that chemo-PTT INMs exhibited apoptosis cell death mechanism. Increased apoptotic mechanism (almost doubled) as well as a minimal necrotic effect was observed for INMs than free chemotherapeutic drugs using flow cytometry. Collectively, these results highlight that the three INMs—[DOX][ICG], [DOX][IR820] and [DOX][IR783]— are promising chemo-PTT combination drugs with great synergy. Based on the promising results generated, further studies is underway for validation prior to in vivo.

### Study 2

Herein, a detailed study is performed to investigate the internalization mechanism of three distinct INMs ([DOX][IR820], [DOX][IR783], [DOX][ICG]) and parent DOX in MCF-7 breast cancer cells at different temperatures. The changes in the INM's cellular uptake mechanism towards MCF-7 will aid us to understand the effect of DOX's counterions. Endocytosis mechanism of the INMs is further examined through assessment of cellular uptake in the presence and absence of different inhibitors using confocal fluorescence microscopy and cell viability assay. It is anticipated that INMs physicochemical properties such as nanoparticle sizes, surface charge and composition play a key role in endocytosis process which consequently alters their transport mechanisms as opposed to free DOX. Lastly, a time-dependent subcellular localization study of the three INMs, relative to parent DOX was performed to gain more insight about the enhanced therapeutic activity of the INMs.

### EXPERIMENTAL

Chemicals: Methyl-beta-cyclodextrin (MβCD) (Lot #A0374873) was purchased from Acros Organics (New Jersey, NJ). 4-(2-Aminoethyl)benzenesulfonyl fluoride hydrochloride (AEBSF) (Lot #GVPZD-TX) was purchased from Tokyo chemical industry (Tokyo, Japan). Filipin III (Lot #0000135711), chloropromazine hydrochloride (Lot #MKCN7684), chloroquine diphosphate salt (Lot #127F-0833), 5-(N-ethyl-N-isopropyl) amiloride, phosphate buffered saline (PBS, pH 7.4) and dimethylsulfoxide (DMSO) were purchased from Sigma Aldrich. Imipramine hydrochloride (Lot #M09J020) was purchased from Thermo scientific, USA. Coverslips, paraformaldehyde, glycine, bovine serum albumin (BSA), saponin, glycerol, 1,4-phenylenediamine, sucrose, were purchased from Fisher Scientific (Hanover Park, IL). All chemicals were used as received. Triply deionized water (18.2 MO cm) was obtained using Purelab Ultrapure water purification system (ELGA, Woodridge, IL). 4′,6-diamidino-2-phenylindole (DAPI), both primary LAMP-2 (H4B4) sc-18822 (Lot #L1720) and secondary antibody m-IgGk BP-CFL 488 sc-516176 (Lot #D1922) for lysosome tracker were purchased from Santa Cruz biotechnology (Texas, USA).

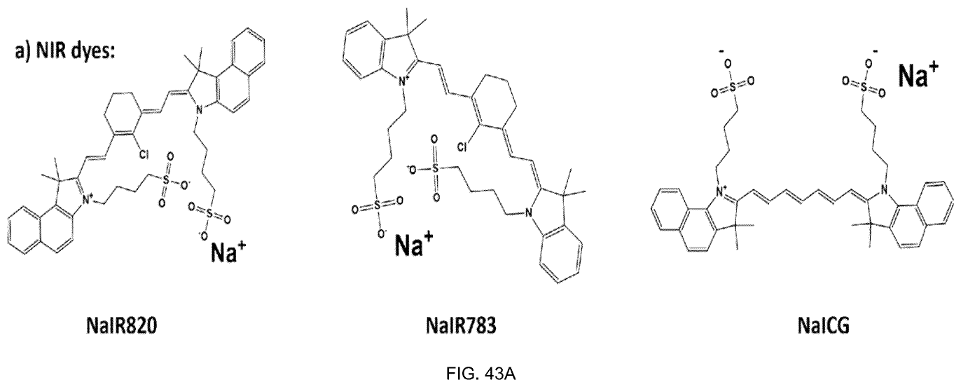

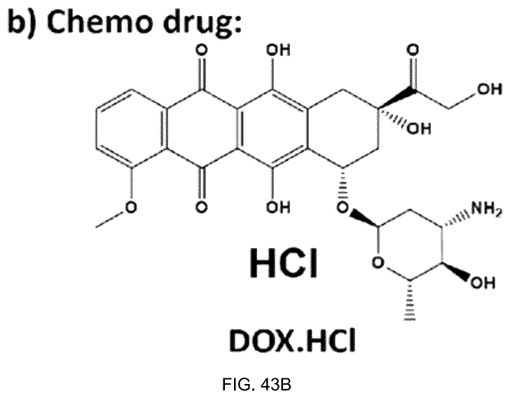

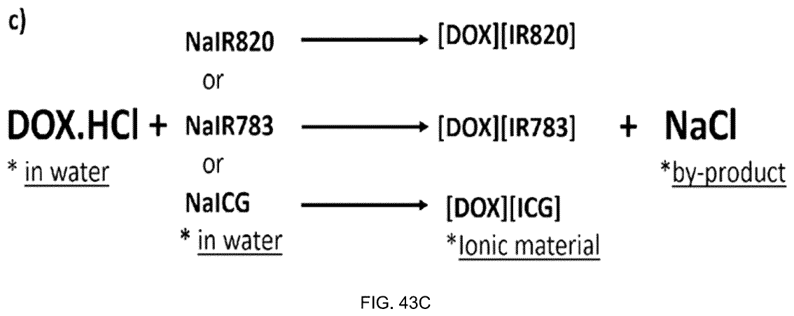

Synthesis of IMs and INMs: Three chemotherapeutic-photothermal (chemo-PTT) combination drugs (IMs) were synthesized using a simplistic, rapid, economical, one step ion exchange method according to previously reported protocol. Briefly, for synthesis of [DOX][IR820]IM, 1:1 mole ratio of DOX and NaIR820 were separately dissolved in water. Aqueous solutions of both DOX and NaIR820 were combined, and mixture was stirred for 24 hr to ensure complete ion exchange reaction. The resultant mixture was centrifuged at 3800 RPM for 5 min and the supernatant was removed to recover precipitate of [DOX][IR820]IMs. The precipitate was washed three times with water to remove the by-product (NaCl). The resultant drug [DOX][IR820]was lyophilized to remove moisture from IMs before performing further studies. Since NaIR783 and NaICG are also water-soluble dyes, similar protocol was followed to synthesize the chemo-PTT combination drugs for [DOX][IR783] and [DOX][ICG]IMs respectively. FIGS. 43A-43C show the three different NIR dyes, chemo drug and synthesis scheme for the different chemo-PTT combination IMs.

INMs were prepared by facile reprecipitation method in cell media from IMs for in vitro study. Briefly, a stock solution of the chemo-PTT combination IMs was initially prepared at 1 mM concentration in DMSO. Next, a small volume of the stock solution was added dropwise to a glass vial containing cell media present in an active sonication bath. The sample was subjected to sonication for 5 min and further stabilized for 20 min before performing any experiment. A Zetasizer pro red, Malvern instrument (Malvern Worcestershire, United Kingdom) was used to determine the hydrodynamic diameter of the INMs in DI water using the dynamic light scattering (DLS) method.

Cell culture: In vitro experiments were performed using MCF-7 breast cancer cell line obtained from the American Type Culture Collection (ATCC, Manassas, VA). A monolayer of cells was maintained in an incubator at 37° C. and 5% CO₂ in complete cell media. MCF-7 cells were cultured in DMEM supplemented with FBS (10% v/v) and antibiotic solution containing penicillin/streptomycin (500 units/mL). When cells reached the desired confluency, they were either subcultured or used for experiments following trypsination and detachment. The detached cells were washed and stained with trypan blue exclusion dye followed by counting using a hemocytometer.

Temperature dependent internalization study of INMs: This experiment is designed to determine the potential mode of entry of the drugs (DOX or INMs) into cells as energy dependent process or passive uptake. The cells were incubated with INMs or DOX at two temperatures, cold (4° C.) and at physiological temperature (37° C.). Active uptake mechanisms such as endocytosis are known to be hindered at low temperatures because it is an energy dependent process. While the passive diffusion cannot be affected by change in temperature. In a typical experiment, 1.2×10⁵ cells per well were plated in a 24 well plate pre-introduced with 12 mm circular coverslips. After 24 hr incubation, the well plate was further incubated at either temperature (4 or 37° C.) for 1 hr before treatment with 5 μM INMs or DOX prepared in cell media. Then the treated cells were incubated again for an additional 1 hr at 4 or 37° C. Then, cells were washed thoroughly with phosphate buffered saline (PBS, pH 7.4) and fixed with 200 μL paraformaldehyde (4%) for 15 min at room temperature. The cells were post treated with DAPI (300 nM), a nucleus strainer for 5 min, washed with PBS and the coverslips were mounted onto microscope slides with 5 μL of mounting media (90% glycerol, 10% PBS with 10 mg 1,4-phenylenediamine). Confocal imaging was performed using a laser scanning confocal microscope (Zeiss, LSM 880), attached to an inverted microscope. An oil immersion objective lens (63×) was used for cell imaging. A diode excitation source of 405 nm was utilized with emission set at 650 nm to view INMs.

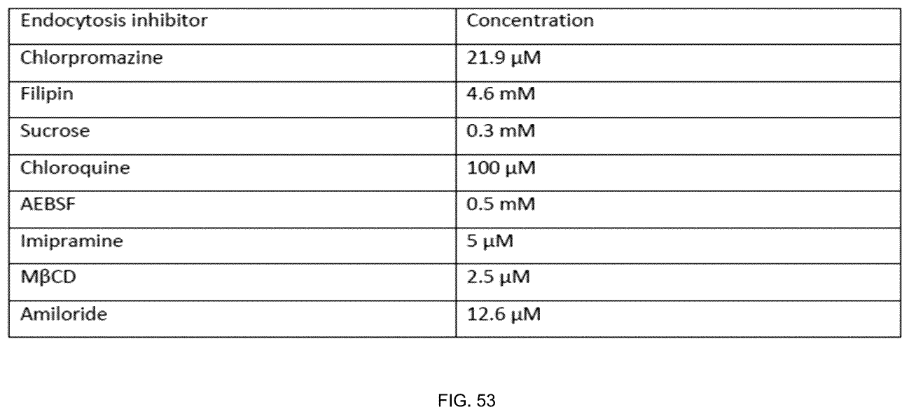

Evaluation of endocytic uptake mechanism: Eight different specific endocytosis inhibitors named Filipin III, sucrose, chlorpromazine, amiloride, imipramine hydrochloride, AEBSF, chloroquine and MβCD were used to investigate the endocytic routes employed by the INMs on MCF-7 cells. All inhibitors stated have been explored and reported to obstruct endocytosis processes such as CME, CVME or macropinocytosis. As a result of the non-selectivity of the inhibitors towards a specific endocytic pathway, multiple inhibitors were tested. In a typical experiment, 1.2×10⁵ cells were seeded on 12 mm circular coverslips pre seated in a 24 well plate and incubated for 24 hr. After the allotted time, the various endocytosis inhibitors including chlorpromazine, filipin III, sucrose, chloroquine, AEBSF, imipramine hydrochloride, MβCD and amiloride prepared in cell media were introduced separately into different wells at their respective concentrations for 2 hr. FIG. 53 reports the various endocytosis inhibitor as well as their concentrations used in this experiment. The excess cell media containing inhibitor was aspirated and the cells were further treated with 5 μM INMs for an additional 1 hr. Next, cells were washed with PBS, and subsequently fixed with 200 μL paraformaldehyde (4%) for 15 min at room temperature. The coverslips were mounted onto microscope slides with 5 μL of mounting media (90% glycerol, 10% PBS with 10 mg 1,4-phenylenediamine). Confocal images were taken to view DOX's fluorescence emission using a laser scanning confocal microscope (Zeiss, LSM 880), attached to an inverted microscope.

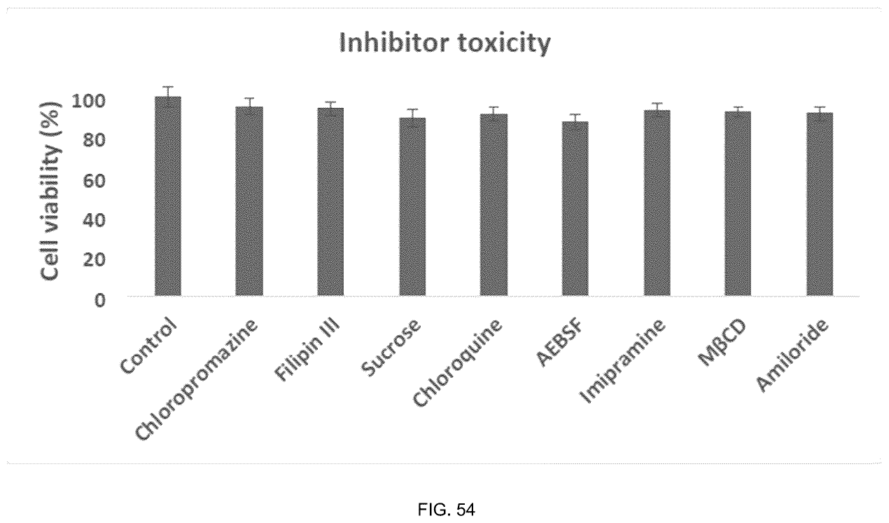

Cell viability study of drug in the presence of endocytic inhibitors: Cells were plated at 1.5×10⁴ cells per well in a 96 well plate and incubated at 37° C. and 5% CO₂ for 24 hr. The toxicity of the inhibitors was investigated with concentrations reported in FIG. 53 and cell viability results shown in FIG. 54. For treatments involving the presence of specific inhibitor, cells were pretreated with various specific inhibitors for 2 hr with indicated inhibitor concentrations (FIG. 53). Cells were washed with PBS and further treated with INMs' prepared in cell media at their half-maxima inhibitory concentration (IC₅₀) and were incubated for 24 hr. Cell viability was assessed utilizing MTT assay. With the use of a microplate reader (Biotek Synergy H1, Winooski, VT), the optical density of MTT-formazan was determined at 570 nm. Each experiment for in vitro studies was performed in triplicate and repeated three times.

Subcellular localization: Localization experiments were designed to investigate the location of the parent drugs and INMs at the various subcellular organelles at different time. We anticipate that the experiments would aid the understanding of improved therapeutic effect of INMs as compared to DOX. In a typical experiment, 1.2×10⁵ cells per well were seeded in 24 well plate previously seeded with coverslips and were incubated for 24 hr. Cells were washed with PBS, treated, and incubated with the 5 μM INMs or DOX prepared in cell media for 1 hr or 6 hr. After the stated time, cells were further washed to remove the uninternalized drugs and fixed with paraformaldehyde for 15 min. Cells were then permeabilized using blocking solution (0.3 M glycine, 10% BSA, 1% saponin in PBS) for 30 min before staining with LAMP antibodies. Cells were incubated with primary LAMP antibody (H4B4) for 30 min at 1:100 dilution according to manufacturer's protocol. The fixed cells were washed repeatedly thrice with PBS for 5 min interval totaling 15 min before the addition of the fluorophore-conjugated secondary antibody (Alexa 488) at 1:100 dilution. Cells were washed with PBS and further treated with 300 nM DAPI for 5 min. Cells were washed with PBS before mounting on the glass slide and imaged using the Laser scanning confocal microscope (Zeiss, LSM 880). Quantitative analysis was performed by creating a region of interest around the whole cell and the nucleus. The total area of the drug content (red channel) in the whole cell and the nucleus was also obtained from the Zeiss LSM880 instrument. The colocalization of the drug with LAMP 2 was also acquired.

### Results and Discussion

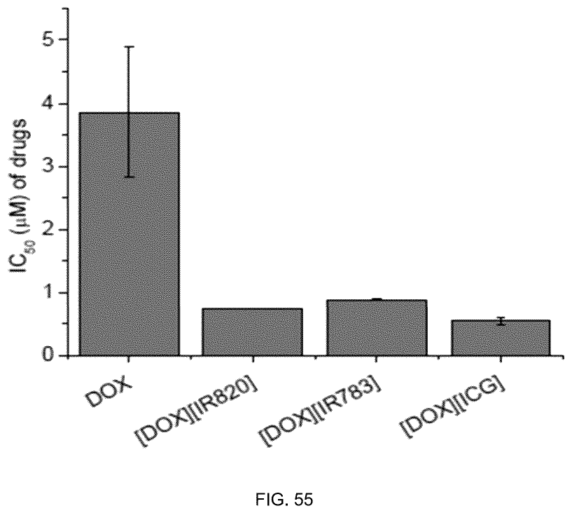

Based on our previous findings, it was observed that the synthesized INMs are spherical in shape.^{[5]} Their hydrodynamic diameter size ranges from 54.1+22.5 nm, 171.1±30 nm and 56±30 nm for [DOX][IR820], [DOX][IR783] and [DOX][ICG]respectively. The cell viability results from our previous study in MCF-7 showed that the three INMs had much lower IC₅₀ values, and are therefore more cytotoxic than DOX. The IC₅₀ values for DOX and various INMs are shown in FIG. 55. Moreover, caspase and flow cytometry analysis revealed that INMs caused more apoptotic cell death than free soluble DOX.

Affirming endocytic uptake for INMs: To understand the enhanced dark toxicity and improved apoptotic cell death mechanisms, the cellular uptake mechanism was studied in detail. Literature reports have confirmed that soluble drugs employ passive transport mechanism to internalize into the cells. However, when these drugs were incorporated into the nanoparticles for drug delivery purpose, the nanoparticles mainly utilized endocytosis mechanism for internalization into the cells. To investigate the potential uptake mechanism of the DOX-based INMs and the parent DOX drug, the cells were incubated with the drug as stated in the experimental section at two different temperature conditions (4° C. and 37° C.). These two temperatures were selected because passive uptake mechanism does not depend on temperature while it is well established that active transport is usually hindered at low temperature. After 1 hr post treatment, cells were examined using confocal microscopy. It was observed that only the parent DOX, the soluble chemotherapeutic drug was internalized into the MCF-7 cells at both temperatures, as evident from the fluorescence emission of DOX (FIG. 44 and green arrow showing uptake at low temperature) inside the cell. This indicates that DOX is being passively up taken by the cell since the DOX uptake is not affected by temperature condition. However, the fluorescence emission of all INMs were observed in the cells at only the physiological temperature (37° C.) condition but not at low temperature (4° C.). This observation proved that these soft carrier-free INMs utilized active transport mechanisms to internalize into the cells. The difference in uptake mechanism could be the reason for enhanced cytotoxicity and improved apoptosis mechanism.

Endocytosis Inhibitor assay: Since INMs employed active transport mechanisms, it is important to investigate the endocytosis mechanisms of all INMs to better understand their improved cytotoxicity towards MCF-7 cells as compared to the parent DOX. Macropinocytosis, CVME, and CME are all size-dependent endocytosis mechanisms, with internalized nanoparticle sizes ranging from 0.2 μm-5 μm, 50-100 nm and less than 200 nm respectively.

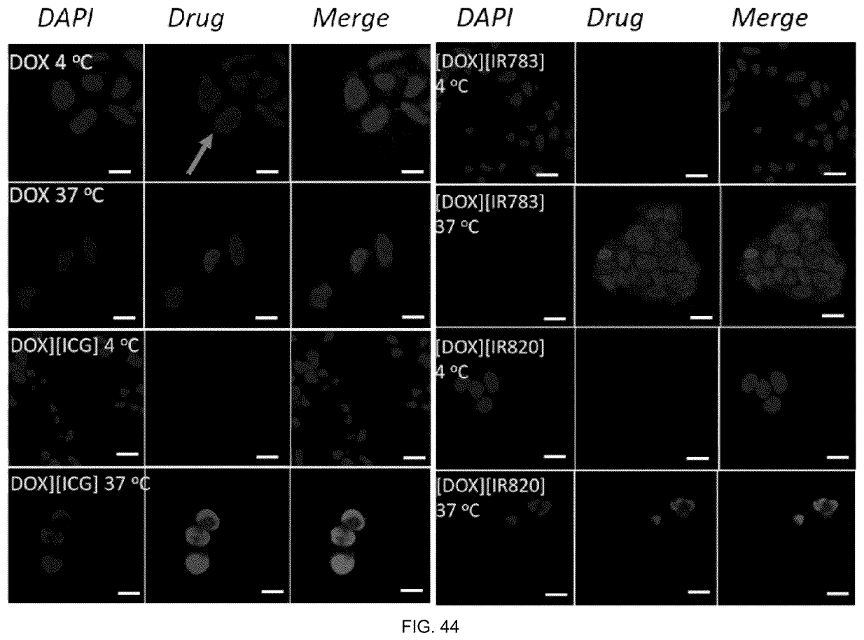

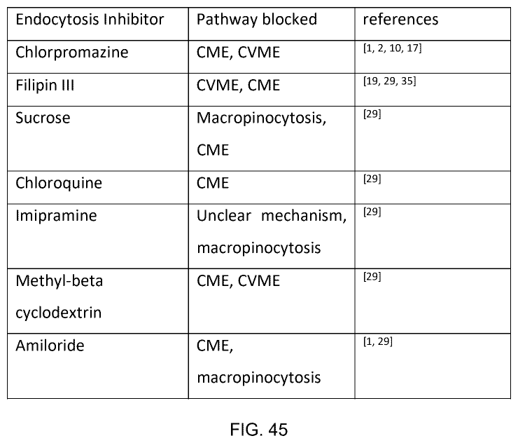

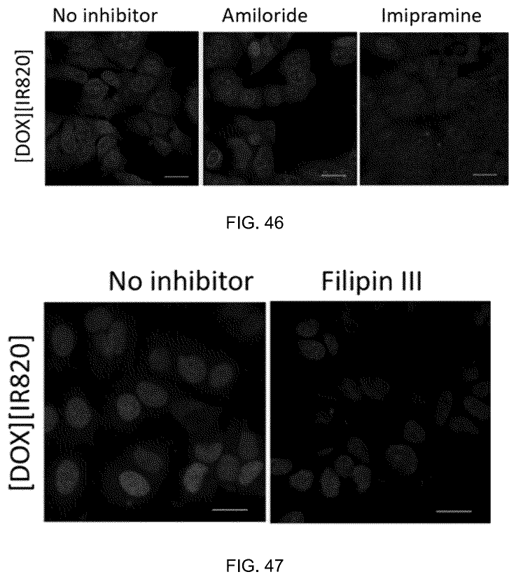

Since not all endocytic pathways can be deduced by using a single inhibitor due to its non-selectivity, therefore more than one inhibitor was utilized to verify the endocytic route of each INMs. FIG. 45 lists the various endocytosis inhibitors that have been studied along with the blocked routes. In addition, the cell viability of the different endocytic inhibitors was also reported in FIG. 53. The results obtained for each INMs are presented one by one. First, the endocytosis path of [DOX][IR820]INM is presented. Amiloride, a macropinocytosis known inhibitor, was first used to examine the macropinocytosis pathway. When [DOX][IR820]INMs were introduced to MCF-7 cells pretreated with amiloride (FIG. 46), the internalization of [DOX][IR820]INMs into the cells was not hindered as evident by confocal fluorescence images showing no significant decrease in the cellular uptake of the nanodrug. This observation was validated with a different macropinocytosis inhibitor: imipramine. As seen with amiloride, imipramine caused no change in fluorescence emission of [DOX][IR820]INMs. These results demonstrate that [DOX][IR820]INMs does not employ macropinocytosis as its mode of entry into the MCF-7 cells.

Next, filipin III inhibitor was used to investigate the CVME process as a possible mechanism for the uptake of [DOX][IR820]INMs. It is well known that filipin III blocks majorly CVME endocytosis pathway with its mode of action focusing on cholesterol binding. Filipin III has also been reported to obstruct the CME pathway. It was observed that the cellular uptake of [DOX][IR820]INMs was slightly reduced in the presence of filipin III inhibitor as shown in FIG. 47. However, a decrease in the fluorescence emission of the drug could imply that the drug is being internalized by more than one pathway. Thus, indicating that the entry of [DOX][IR820]INMs in MCF-7 cells could be CVME dependent in addition to other possible mechanisms such as CME.

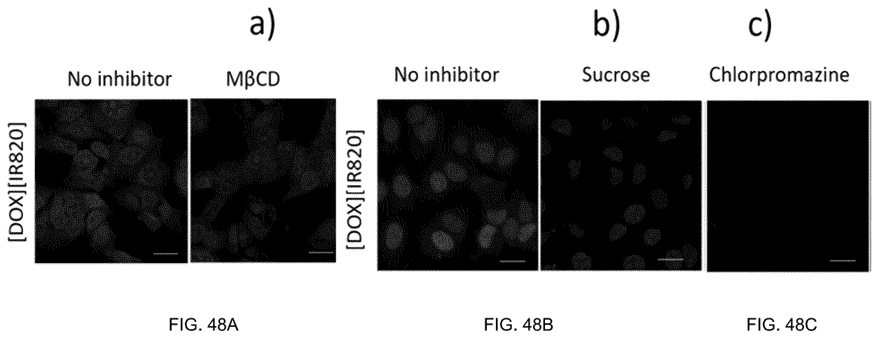

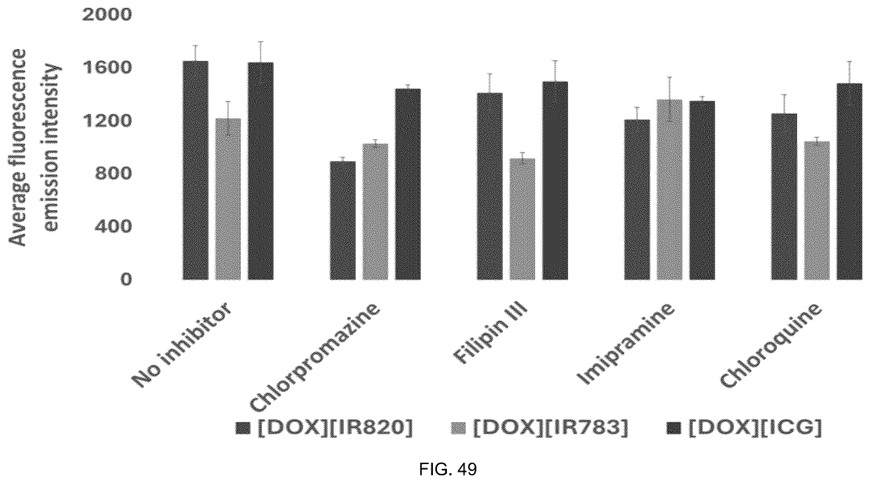

To further investigate whether [DOX][IR820]INMs were actively endocytosed via the CME mechanism, the effect of a CME known inhibitor such as MβCD on drug internalization (FIG. 48A) towards MCF-7 cells was examined. It is important to clarify that MβCD is known to non-selectively inhibit multiple pathways such as CME and CVME. The confocal fluorescence images revealed a slight decrease in the fluorescence emission of the [DOX][IR820]INMs, signifying the possibility of CME and CVME pathways as its mode of entry in MCF-7 cells. CME mechanism was also investigated using sucrose as an inhibitor. Hypertonic sucrose are known to hinder the clathrin coated-pit formation, thereby hindering CME process. It was observed that the uptake of [DOX][IR820]INMs in MCF-7 cells decreased with treatment involving sucrose when compared with the control (FIG. 48B). Next, the cellular uptake of [DOX][IR820]INMs in the presence of chlorpromazine, majorly known to inhibit the CME process, was examined. It was observed that the fluorescence emission of the drug was quenched as depicted by a reduced uptake of INMs (FIG. 48C). We also quantitatively analyzed the average fluorescence emission of the INMs in the presence of CME, CVME and macropinocytosis known inhibitors and presented the bar graphs in FIG. 49. Both Quantitative and qualitative result indicates the likely endocytosis pathway of [DOX][IR820] is CME. Based on the size of [DOX][IR820]INMs, i.e 54.1±22.5 nm it is expected to be internalized via either CME or CVME routes.

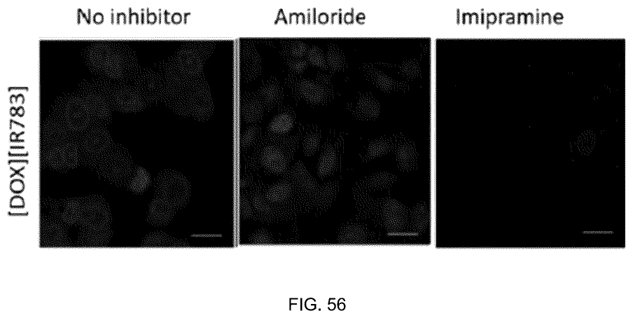

Similar endocytic inhibitors were examined for the endocytosis mechanism employed by [DOX][IR783]INMs in MCF-7 cells. The cellular uptake of [DOX][IR783]was not affected in the presence of amiloride inhibitor (FIG. 56). However, there was a decrease in the uptake of nanodrugs in the presence of imipramine. As stated earlier, there have been some controversies on imipramine's ambiguous mechanism. Nevertheless, other studies indicate that imipramine blocks macropinocytosis route. Based on this result as well as its size (nearly 200 nm), it is possible that [DOX][IR783]INMs is being endocytosed via the macropinocytosis route.

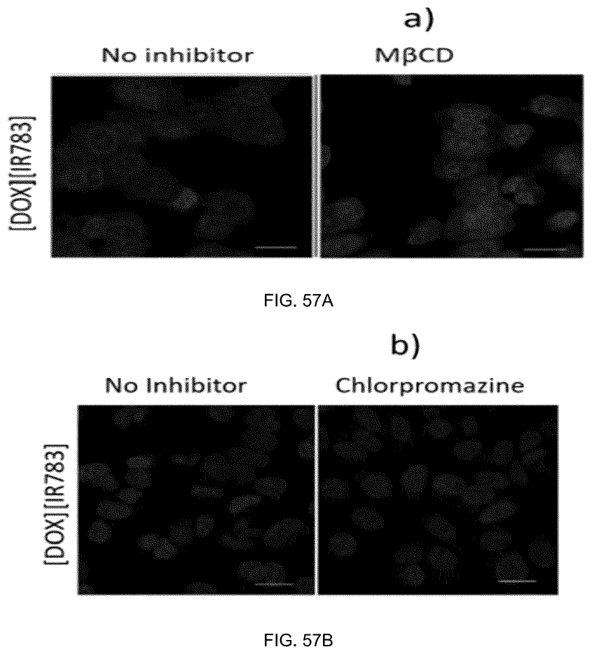

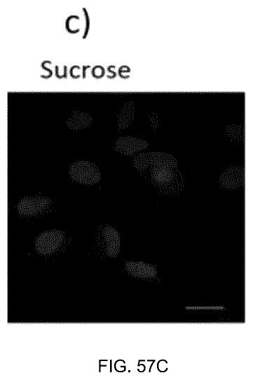

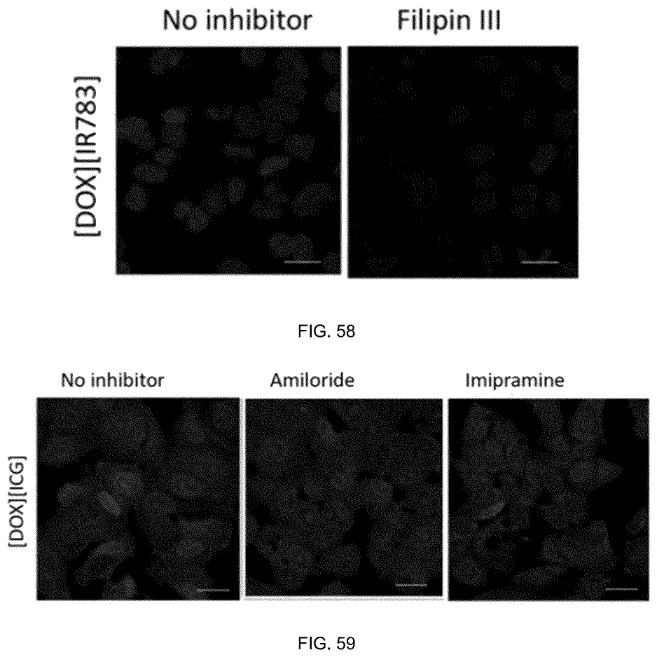

Next, the relationship between [DOX][IR783]INMs and CME process was studied. When MCF-7 cells were pre-treated with MβCD inhibitor prior to [DOX][IR783]INMs, the fluorescence emission of [DOX][IR783]INMs was not altered (FIG. 57A). It is well known that MβCD may also inhibit other processes such as CVME pathway in addition to CME process. In the presence of chlorpromazine, known to majorly impede CME process, there was a slight reduction in the uptake of [DOX][IR783]INM in MCF-7 as shown in FIG. 57B. Although it has been reported that chlorpromazine can inhibit CVME pathway in addition to CME. (FIG. 45). Similarly, when MCF-7 cells pre-treated with sucrose was treated with [DOX][IR783], we observed only a slight decrease in the cellular uptake of [DOX][IR783](FIG. 57C). Hypertonic sucrose is known to non-selectively obstruct both CME and macropinocytosis routes. This ambiguity informed our decision to further investigate the role of CVME process in the internalization of [DOX][IR783]INMs using filipin III, which obstructs both CVME and CME route. Interestingly, the cell uptake of [DOX][IR783] on MCF-7 cells was reduced upon treatment with filipin III, as evident from the images shown in FIG. 58. Based on the nanoparticle size range (171.1±30 nm) and complexity of [DOX][IR783]INMs uptake mechanism, it is evident that [DOX][IR783]INMs employ more than one endocytosis route including macropinocytosis, CME and CVME.

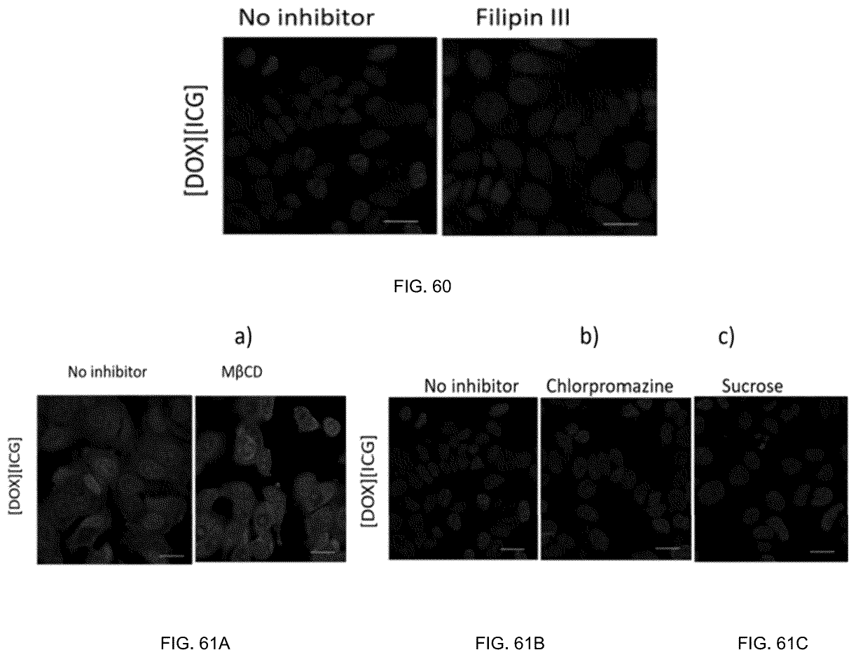

For the endocytic study involving [DOX][ICG]INMs in the presence of macropinocytosis inhibitor, both amiloride and imipramine did not impact the internalization process (FIG. 59). Similarly, upon treatment of [DOX][ICG] with MCF-7 cells in the presence of filipin III (a CVME inhibitor), there was only a slight decrease in the cell uptake of the nanodrug as shown in FIG. 60. The cellular uptake of [DOX][ICG]INMs was not reduced when the cells were treated with MβCD inhibitor prior to the [DOX][ICG] introduction in the MCF-7 cells (FIG. 61). However, confocal fluorescence images revealed that the presence of chlorpromazine and sucrose only marginally decreased the cell uptake of [DOX][ICG]INMs (FIG. 61B-61C). These inhibitors are known to majorly obstruct the CME in addition to CVME pathway. As previously stated, the introduction of a specific type of inhibitor may also lead to the modulation of other pathways alongside their major pathway. For instance, a specific inhibitor such as sucrose known to majorly inhibit CME route, has also been reported to have some off targets effects leading to the uptake of drugs via macropinocytosis due to possible crosstalk between different endocytosis pathways. It is not new that one nanodrug may use two different endocytosis pathways for cell internalization process. In addition, Sousa et.al reported that macropinocytosis is a non-specific cargo uptake mechanism and do not solely depend on the size of the nanoparticle.

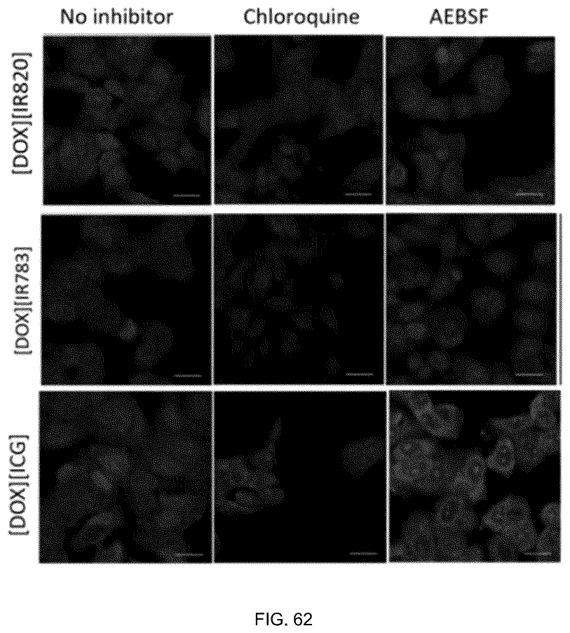

Detailed examination of the confocal images revealed that the internalization pathway of the three INMs at least involved CME and other additional endocytosis pathways. It is interesting to note that simply by changing the counterion, endocytosis mechanism can be tuned. When particles are endocytosed via CME process, it is anticipated that the nanoparticle will fuse with the lysosome at a later stage of the process, following the development of the late endosome. Therefore, to further comprehend the internalization processes of these DOX based INMs in MCF-7 cells, the role of specific lysosomal inhibitors and lysosomal enzyme in MCF-7 cells were examined upon treatment with the INMs. Chloroquine is a commonly known lysosomal inhibitor that reduces lysosome activity and inhibits the CME process. On the other hand, a lysosomal enzyme like AEBSF can increase lysosomal activity. When MCF-7 cells were treated independently with the three INMs in the presence of chloroquine, there was a significant decrease in the fluorescence emission of the three INMs as compared to control (FIG. 62). This suggests the possible pathway for [DOX][IR820], [DOX][IR783] and [DOX][ICG]INMs internalization involves the CME process. In the case of AEBSF lysosomal enzyme, it was observed that the presence of AEBSF did not cause a decrease in the cellular uptake of the three INMs towards MCF-7. This would mean that the increased lysosomal activity led to enhanced lysosomal fusion (FIG. 62). Thus, confocal imaging result signifies that the internalization process of [DOX][IR820] and [DOX][IR783]INMs occurs via the CME pathway.

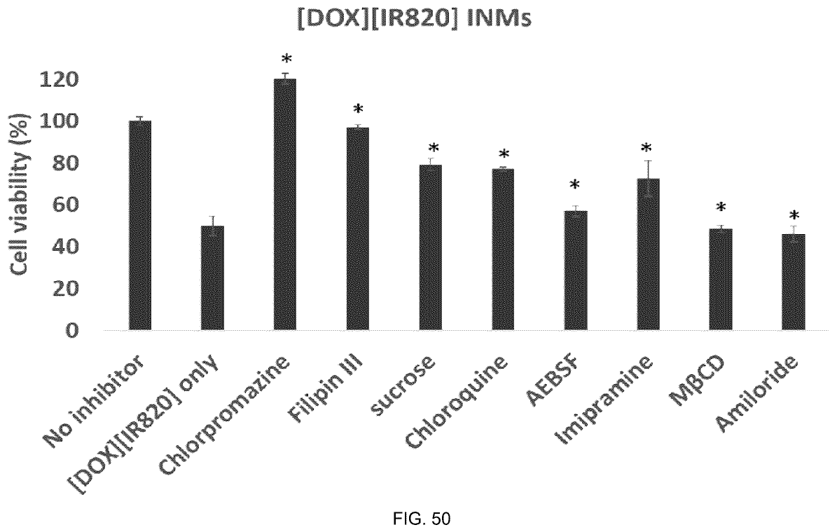

In vitro cell viability study in the presence of endocytosis inhibitors: The complicated uptake mechanism employed by some of the INMs led us to validate the results using a quantitative cell viability assay. We have investigated the toxicity of DOX-based INMs on MCF-7 breast cancer cells in our previous report. A change in cell viability results in the presence of an endocytosis inhibitor would depict possible pathway for the uptake process. Cell viability results shown in FIG. 50 revealed that the toxicity of the [DOX][IR820]INMs decreased in the presence of CME related inhibitors (chlorpromazine, sucrose, and chloroquine) in relation to the control (drug only without inhibitor). All these CME inhibitors decreased the uptake of [DOX][IR820]INMs. MCF-7 cells pre-treated with chlorpromazine prior to [DOX][IR820] incubation resulted in the lowest cellular uptake of the nanodrug when compared to treatments involving other inhibitors. This result signifies the cell growth due to complete obstruction of the CME pathway caused by chlorpromazine. This result agrees with the confocal fluorescence image earlier discussed for [DOX][IR820]INMs shown in FIG. 48C. Cell viability results involving filipin III treated MCF-7 cells, incubated with [DOX][IR820]INMs similarly showed a decreased cellular uptake. This data signifies the inhibitory effect of CVME, in addition to the major CME pathway employed by the nanodrug as means of internalization in MCF-7. However, AEBSF treated MCF-7 cells showed no significant difference in the cellular uptake of [DOX][IR820]INMs in relation to the drug treatment only. This result is consistent with the fluorescence images shown in FIG. 62. In the presence of macropinocytosis related inhibitors such as imipramine and amiloride, we observed some inhibitory effects with imipramine only but not with amiloride. The effect exhibited with treatment involving imipramine could possibly be due to its unclear mechanism of endocytosis previously reported. Therefore, both quantitative confocal fluorescence imaging and qualitative cell viability results demonstrated the primary involvement of CME in addition to a secondary CVME for [DOX][IR820]INMs in MCF-7 cells.

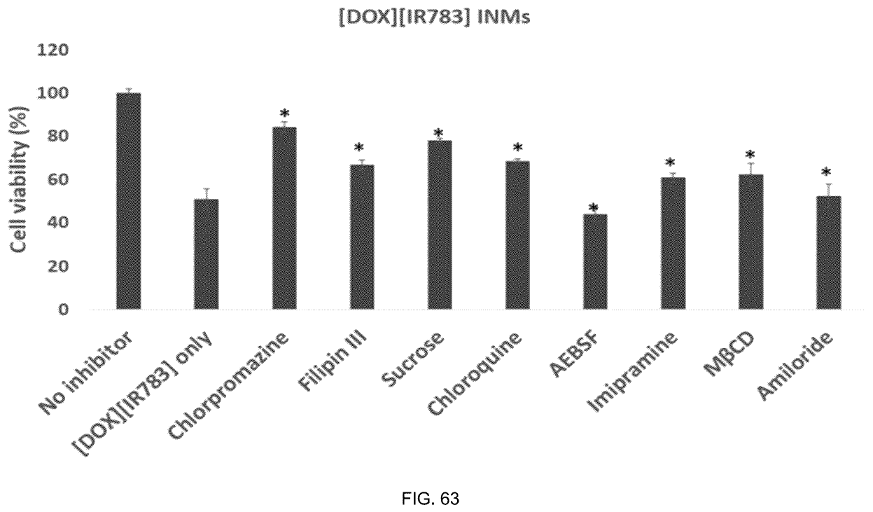

Cell viability results for MCF-7 cells treated with [DOX][IR783]INMs in the presence of different endocytic inhibitors are reported in FIG. 63. These results revealed that the presence of CME-related inhibitors (chlorpromazine, sucrose, chloroquine, MβCD) reduced the cellular uptake of [DOX][IR783]INMs in MCF-7, thus rendering the nanodrug less toxic. Thus, the results confirmed that [DOX][IR783]employs the CME pathway as a means of entry in MCF-7 cells. We also observed that filipin III also hindered the internalization of [DOX][IR783]INMs and thus led to an increased cell viability. The lowest cellular uptake was exhibited with the treatment involving [DOX][IR783]INMs in the presence of macropinocytosis inhibitors such as imipramine and amiloride, possibly indicating partial cellular uptake via the macropinocytosis route. These results demonstrated that [DOX][IR783]INMs employed the CME and CVME pathway for internalization in MCF-7 cells.

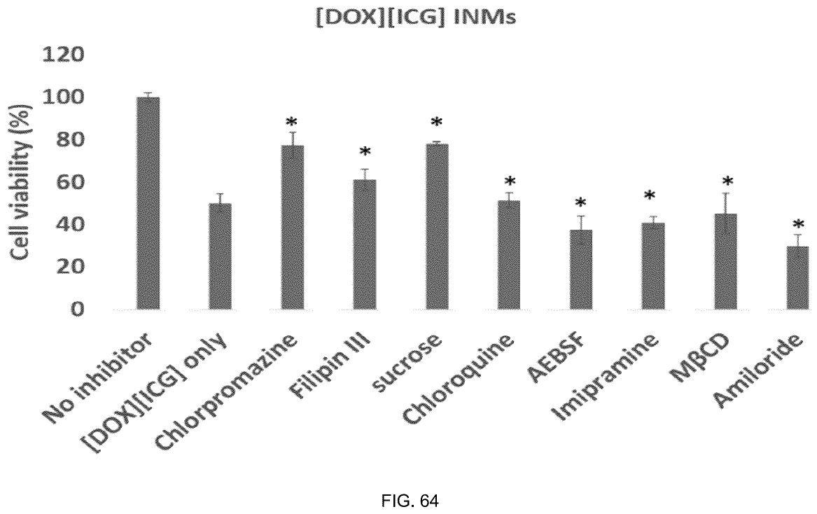

When MCF-7 cells, preincubated with the different endocytosis inhibitors, chlorpromazine, sucrose, and chloroquine were post-treated with [DOX][ICG]INMs, we observed a reduction in the cellular uptake of [DOX][ICG] in comparison with the [DOX][ICG]treatment in the absence of these endocytosis inhibitors (control) (FIG. 64). Similarly, the presence of filipin III also resulted in increased cell viability results. However, treatment involving other inhibitors such as imipramine, AEBSF, MβCD and amiloride did not significantly alter the cellular uptake in MCF-7 cells. Therefore, the study demonstrated the partial involvement of CVE or CVME in the uptake of [DOX][ICG] by the MCF-7 cells.

Subcellular localization: After identifying endocytosis pathway for each INMs, it is important to investigate the final location of the drug in the subcellular organelles which can provide a better insight into the improved toxicity of the drug. In the subcellular localization study, the parent DOX drug was also included as control to investigate any changes of the DOX-based INM's target in the cells. To investigate the subcellular localization of the combination drugs in MCF-7 cells, confocal imaging was performed. The localization of the soluble chemo drug and the various chemo-PTT INMs within the cell was investigated at different times (1 and 6 hr). Confocal imaging is effective for observing drug's localization at the subcellular level based on the drug emission, which could also reveal the potential organelle subjected to damage and ultimately responsible for cell death.

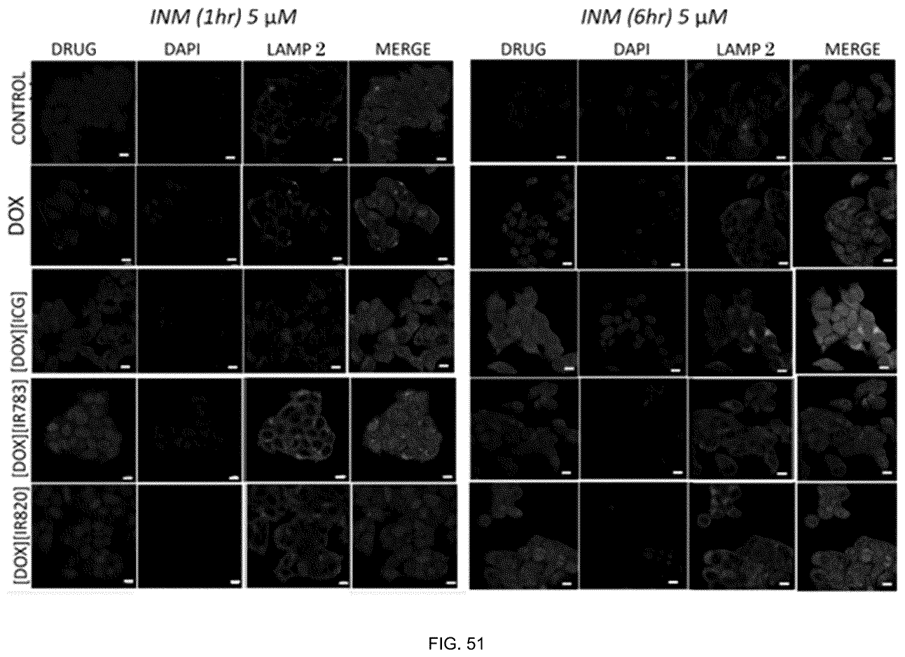

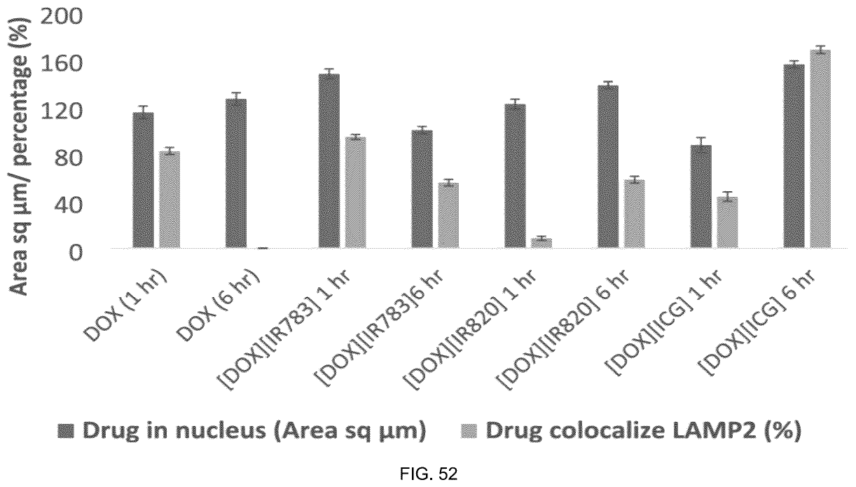

Qualitative and quantitative confocal imaging results presented in FIGS. 51-52 revealed that DOX was mainly concentrated in the nucleus for the first hour as evident by the overlap of the DOX's red emission with DAPI. The DOX remained localized in the nucleus even after 6 hr of drug incubation (with no drug colocalized with LAMP 2), corroborating earlier research about the DOX targeting the nucleus. It is well established that DOX inhibits topoisomerase II which hinders DNA repair processes and causes apoptosis. [DOX][IR820]INM was observed mainly within the nucleus for the first hour of incubation. It's interesting to note that [DOX][IR820]INM's location changed overtime. After 6 hr, it was observed to be present in both the nucleus and the lysosome, as evident by the INMs intense emission overlapping with the LAMP 2 antibody. Similarly, throughout the first hour [DOX][IR783] and [DOX][ICG]INMs primarily targeted the cell's nucleus. As incubation time increased, the [DOX][ICG]INMs targets shifted to the cell's lysosome. However, the cellular uptake of [DOX][IR783]decreases with time due to the nanoparticle morphology. [DOX][IR783]cellular uptake was more in the first hour as compared to 6 hr (as evident from both qualitative and quantitative result (FIGS. 51-52). A shift in location of the INMs indicates the effect of DOX counterion and its nanoparticle morphology that impacted its subcellular localization. The cellular uptake and changes in subcellular localization of INMs as compared to parent DOX is attributed to the improved toxicity as well as enhanced apoptosis mechanism of the INMs. According to reports, lysosome destruction triggers apoptosis by releasing proteolytic enzymes into the cytoplasm. It is important to note that all confocal images were recorded at DOX emission wavelength. Unfortunately, unavailability of NIR confocal imaging facilities restricted us to track the NIR dyes in the INMs. Although previous research reports the localization of cyanine dyes such as NaIR783 within the lysosome. It is possible that NIR counterions are dragging the DOX towards lysosome and improving the apoptosis cell death mechanism of the overall drug. Consequently, the location of the drug in both nucleus and lysosome can enhance the therapeutic effect on the cancer cell. In addition, these results also demonstrated the stability of INMs in the cells since DOX emission in INMs changed over time while the parent DOX stayed in the nucleus only. Relative to DOX, Chemo-PTT INMs offer the benefits of multimodal organelle targeting that not only enhanced their toxicity but also their apoptotic cell death mechanisms. These results could be tremendously important towards addressing the side effects of the DOX. In future, we will present a more detailed study towards different cell lines and organelles to explore the full potential of INMs.

### Conclusion

In summary, energy dependent active transport mechanisms are being employed by three distinct INMs towards MCF-7 cells as opposed to free soluble DOX that exhibited passive uptake. The alteration in INMs internalization process is attributed to the effect of DOX counterions and nanoparticle morphology. INMs employ CME as their primary means of internalization pathway in addition to other secondary endocytic pathways such as CVME. [DOX][IR820]INMs showed principal internalization via CME due to the drastic quenching of the fluorescence emission of the drug as well as increased cell viability of the cells in the presence of CME inhibitors. While [DOX][IR783]employed CME, CVME and macropinocytosis pathways as a means of entry into MCF-7 cells, [DOX][ICG]exhibited a more complicated endocytic mechanism with partial internalization using the CME or CVME route. Time dependent subcellular studies for all INMs revealed that the INMs are concentrated in multiple organelles over time (nucleus and lysosome) as opposed to soluble DOX which majorly localizes in the nucleus. This dual organelle targeted INMs provides a promising strategy to improve the therapeutic activity of the drug towards cancer cells.

### Study 3

### In Vivo Studies

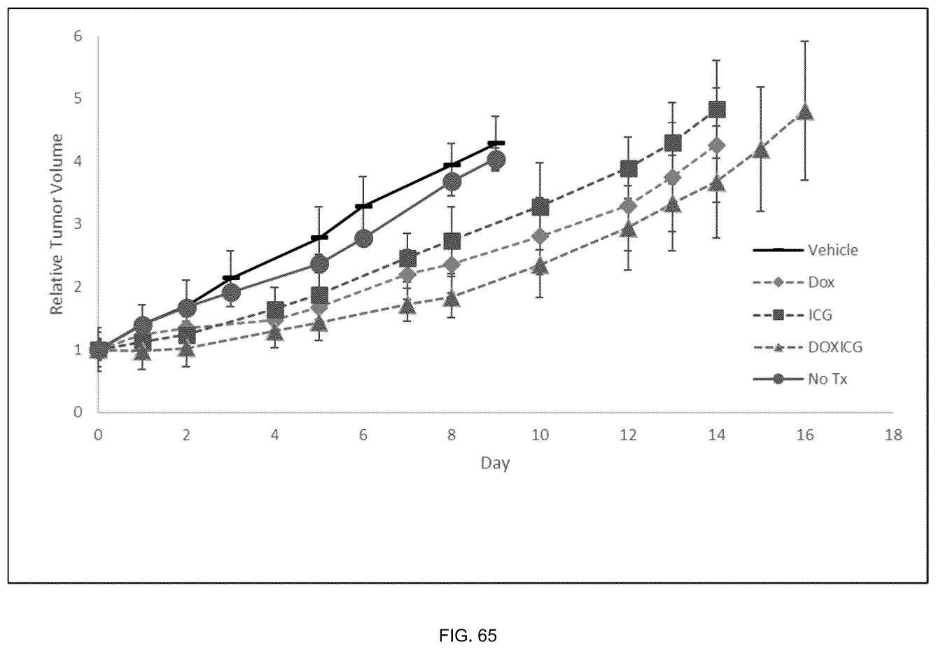

The preliminary in vivo studies also demonstrated that the theranostic [DOX][ICG]CNMs exhibited great toxicity and bioimaging potential. Moreover, when mice (5 mice per group) were treated with parent chemo (DOX) and NaICG as well as with [DOX][ICG]CNMs, then CNMs exhibited the best performance to treat tumor cells as compared to their respective parent compounds in dark (FIG. 65). In this experiment, we used a single dose of 0.025 mg/Kg for each drug. It means we used very less molar concentration of CMNs as compared to their parent respective compounds. Thus, using multiple doses and same molar concentration would aid to attain the better performance of the CNMs in comparison to their respective parent drugs. As we learned from the in vitro studies that dark and light toxicity as well as fluorescence quantum yield are dependent on counterion of CNMs, therefore, we plan to investigate bioimaging capability along with the dark and light toxicity under irradiation for all three CNMs in vivo to investigate the effect of counterion.

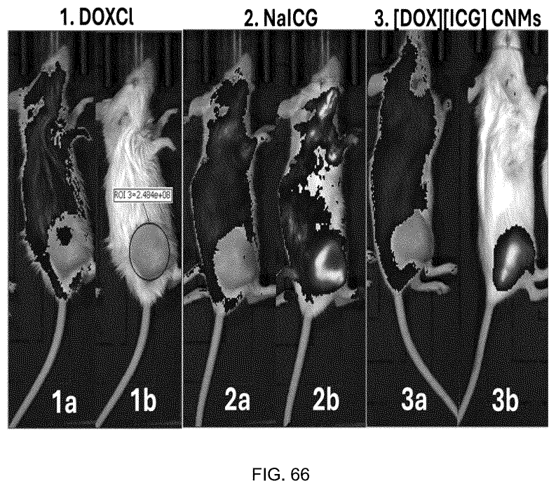

In the preliminary studies performed for bioimaging and biodistribution using IVIS spectrum, we used IT as well as IV injection. In this experiment, we used three mice per group. After 24 hours of IT injection, fluorescence images of mice were recorded that indicate that [DOX][ICG]CNMs mainly reside in tumor whereas the NaICG spread out all over the body (FIG. 66). Interestingly, the fluorescence emission images of the harvested organs recorded after 24 hr of IV injection also revealed that the parent DOX and specially NaICG is mainly located in liver while the [DOX][ICG]CNMs is the only compound which mainly localized in tumor as well as in liver (FIG. 67). Both IT and IV injections results are very promising and indicated that these carrier free nanomedicines exhibited high uptake by cancer cells as compared to the normal cells which is attributed to EPR effect.

The present invention has been described with reference to certain preferred and alternative embodiments that are intended to be exemplary only and not limiting to the full scope of the present invention as set forth in the appended claims.

We claim:

1. A compound for treating cancer comprising doxorubicin electrostatically attached to indocyanine green, IR783, or IR820.

2. A method of treating cancer in a subject in need thereof, comprising the steps of: (a) administering to the subject a pharmaceutically effective amount of a compound comprising doxorubicin electrostatically attached to indocyanine green, IR783, or IR820; and (b) exposing the subject to a near-infrared light.

3. The method of claim 2, wherein the step of exposing the subject to the near-infrared light comprises exposing the subject to the near-infrared light for one minute for three consecutive days after the step of administering to the subject a pharmaceutically effective amount of the compound.

4. The method of claim 2, wherein the step of exposing the subject to a near-infrared light comprises using a laser.

5. The method of claim 4, wherein said laser has a power of 0.5 Watt/cm².

6. The method of claim 4, wherein said laser has a power of 1 Watt/cm².
